SEQUENCE LISTING (82 sequences)

SEQ ID NO: 1 - DNA, 2904 bp, Physcomitrella patens
atggagggctctggggacaagcgacatgagaatttggacgaggatgggtacaactggcatgcgcagtcggtggaatccgtgagcaaggcgttggggacgaatccaaacctgggcgtgtcggatgggcggagtgcagagctgctgaagcagcacggttacaacgagctgaaggggcaggctggggtgaacccatggaagatcttgctgcggcaggtctcgaacggcctgacggccgtgcttgtcgtcgctatggttgtatcctttgcagtgaaggattatgccgaggcgggggttctggtaattgtgattgcgttcaatacaatcgtggggtttgtgcaagaataccgcgccgagaagaccatggatgcattgcggaaaatggcgtcaccctcggcgaaggtgattcgagacggaagccatcaccgaatctcgagcagggatgtggtgcccggggacttgttgacgttcgaggtcggggatgttgtacctgctgactgtcgtctgattgaggttctgaacctggaagtcgacgaggcgttgctgacaggagaggctgtgccttctttgaagactgtgcagccgatcggggggaaggatgtctcgattggggaccgcaccaacatgtcatactcaagtactacggtggtgaaggggagagggaaggcgattgtggtgagcacagggatgtctaccgagattggaaagatttccaaggccatcaacgagacgaagacgcagtccactcctatgcagaggaagcttaatctgatggcctacatgctcctcgcatttgcgctgctgcttgcgctgattgtgtttgctgtaaacaagtttaactttagtactgaggttgtgatttacgccatcgcgctctcgattgctatcattcctgaggggctgattgcagtcatcacgattgtacaagcgctaggcgtgcggcgtatggccaagcagcatgcattggtgaggaaactggtggcgttggaatcgcttcaagcagtgaccaatatctgctcggataaaacgggtacgctgacggagggaaagatggtggtgacgaacgtttggctgccgggacatgagtctgagtacattgtcgctgggcaagggtacgagactgttggcgacttgtccacttcggcgggtgtcgctgtagttagatcagcggcgctggaggatgtgaattatcgattgctggtggagtgctgtgccttgtgcaacacggcgaacatcgtcgaggcatccgagggtaaagtgtggggagaccccactgagatcgccctccaagtgttcgcatataaaatggagatgggtatgcccattctgcgtaaacacaaggaactcgttgaggaatttcctttcagctcagacacgaagcggatgagcatggtatgccagactgagtcgggaaacttcttagagattttcacgaagggttctgaagttgtgctcagtatttgcgacaacgtgatggatcggactggagacatacatagcatctctggggacgaggggttcttgaagctcgtctcaacgcaacaggaggagatggcaaagcaaggtttaagggttctagtgctcgcgtacgggcaagtatcggagagatccatcggcaagcccctttcaaagtgggagcgtaacgacgctgagaaaagcttgacattccttggtttggttggaatcagagacactcctcgggtcgagtctgagcaatctgtgcgcaattgtcaccgcgccggtattacagttcacatgctcaccggagaccataaagccaccgctatgtcgattgcaaaggaagtgggaatcatagaggaaccacatgggtctgagatagcgaatggtaatgaaatcgtccccttgtcagcatctgtcatgaccgcgacagaatttgaccaactgactgatgagcaagtggatgctctggtcgatctccctcttgtaattgcgcggtgcacaccctcaacgaaggtgagaatgatcgacgcgctccaccggagaaagaaattcgtggccatgaccggcgacggggtgaacgacgcgcccagtttgaagaaggccgatgtgggaatcgccatgggcgcaggtagtgatgtggccaagacaagcagtgacatcgtgctcacagacaacaacttcgcaaccatagtacaagccatcggcgaagggcggaggattttctccaacatcaagaagttcgtgttgcacctgctgagcactaacgtcggacaagtgatagtgctactgataggtttggctttcaaggatcggacgggcacttccgtctttccactatccccagtgcagattctctttctgaatctcgtcacgggaacaccgcctgccatggctctgggaattgagccggcctcctcttccgtgatgcaagtccctccccacgtcaaaggacttttcactgtggagctcatcatggacatcttcattttcggcaccttcataggcatcctcgccctggcatcatgggtgctcgtcatctaccccttcggaaactccgacctcgccactctctgcaacaccaccgccaacttacaagaatgctccaccatcttccgcgccaggtccacagttcaactgtccttcacgtggatgatcctcttccacgcttacaactgccgccacctccgtgccagtttgctcactgctgagggaggcggcgcctcgcggttcttctccaacaaagttttggttgcgagtgtgttcatcggtgctcttctgcccatccccaccatctacatcggcacgctcaacacggaagtcttcaagcaggaaggcattacctgggagtggatcatcgtgatcgtctcggtgttcgtcttcttcttgctatcagagttttacaagctgcttaagcgtcgcttcattaaaactccctacaacatgtga

SEQ ID NO: 2 - protein, 967 aa, Physcomitrella patens
MEGSGDKRHENLDEDGYNWHAQSVESVSKALGTNPNLGVSDGRSAELLKQHGYNELKGQAGVNPWKILLRQVSNGLTAVLVVAMVVSFAVKDYAEAGVLVIVIAFNTIVGFVQEYRAEKTMDALRKMASPSAKVIRDGSHHRISSRDVVPGDLLTFEVGDVVPADCRLIEVLNLEVDEALLTGEAVPSLKTVQPIGGKDVSIGDRTNMSYSSTTVVKGRGKAIVVSTGMSTEIGKISKAINETKTQSTPMQRKLNLMAYMLLAFALLLALIVFAVNKFNFSTEVVIYAIALSIAIIPEGLIAVITIVQALGVRRMAKQHALVRKLVALESLQAVTNICSDKTGTLTEGKMVVTNVWLPGHESEYIVAGQGYETVGDLSTSAGVAVVRSAALEDVNYRLLVECCALCNTANIVEASEGKVWGDPTEIALQVFAYKMEMGMPILRKHKELVEEFPFSSDTKRMSMVCQTESGNFLEIFTKGSEVVLSICDNVMDRTGDIHSISGDEGFLKLVSTQQEEMAKQGLRVLVLAYGQVSERSIGKPLSKWERNDAEKSLTFLGLVGIRDTPRVESEQSVRNCHRAGITVHMLTGDHKATAMSIAKEVGIIEEPHGSEIANGNEIVPLSASVMTATEFDQLTDEQVDALVDLPLVIARCTPSTKVRMIDALHRRKKFVAMTGDGVNDAPSLKKADVGIAMGAGSDVAKTSSDIVLTDNNFATIVQAIGEGRRIFSNIKKFVLHLLSTNVGQVIVLLIGLAFKDRTGTSVFPLSPVQILFLNLVTGTPPAMALGIEPASSSVMQVPPHVKGLFTVELIMDIFIFGTFIGILALASWVLVIYPFGNSDLATLCNTTANLQECSTIFRARSTVQLSFTWMILFHAYNCRHLRASLLTAEGGGASRFFSNKVLVASVFIGALLPIPTIYIGTLNTEVFKQEGITWEWIIVIVSVFVFFLLSEFYKLLKRRFIKTPYNM

SEQ ID NO: 3 - DNA, 3865 bp, Physcomitrella patens
atggtcgacatccgagagttgatggaggtaccatgcgacgaatcatgctcaaaccatagcaagtccttcgaggaagttatcaaggttctagacagcaattcggagttggggctctcgaatgccaaagctgagcgattgctgaagcagtacggtcgcaacgagctcaagggtcagggggcagtgaacccatggaaaattttgcttgcgcaagtggccaacggcctgacggccgtgctgacaatcgctatggtggtgtctttcgccgtaaaagactacggcgaaggaggagtgttggtgctagtgattgcattcaacaccatcgttggattcatgcaagagtatcgcgcggagaaaaccatggatgccttgcgaaagatggcgtcgccgtcggcgaaggtcatccgagagggtattcagcaaagaatctccagtacggatgttgttccaggagatgtgctgactttcgaggtcggcgacattattcctgcagactgccgactgatggaggtcctgaatcttgaagttgacgaggctttgctcactggtgaatctgtaccttcgatcaagctcgtagagccaattctgggcaaggatgtctcgattggcgatcgcatcaacatggtgtactcgagcaccacggtggcgaagggaagaggtcgagcaatcgttgcatccaccgggatgtccactgagattggaaagatttccaacgcaatcaatgaaacacctgcgcaattgactctcttgcagcggaggcttaacatgatggcatacgtgctgatggtgatcgcgctcatcttagcactgattgtttttgctgtgaacaaatttgattttaacacggaaataattatctacgccatcgccctcgcaattggtgtcatcccagaggggcttatcgcggtcattaccattgtgcaagctctcggcgtacggcggatggccaagcagcatgcactggtgcggaagctggtagctttagagtccctgcaggcggtgacaaacatctgctcagacaagaccggcactttgacgcagggcaagatggtggtgacgaatgtttggttccctggacacgactccgagtacatcatctccggccagggatacgagaccaaaggcgacatttcggctcagggccgtgctatcgccacagagacagcgctcgaggacttgaatttccggaagctggtggagtgctgcgcgttatgcaacacggcaaacatcgttgaggcttcacagggcaacgtgtggggggatccaactgagattgctctgcaggtgttcgcgtacaaaatgcagatggggaagcctatcctgcgcaagaccaagaagccggttgaggagttccccttcagctccgacaccaagcggatgagcatggtttacctaacaaaactcgaagacgtgcttgaagtttattcaaagggggcagaggttgtgctgacaatctgcgacgatgtgatggaacggacggggaacctcaggaacatcagcgaggacggtgagttcatgaagaatgtgactgtacaacaagaggagatggcgaaacagggtttaagggttttagtgatagcctacagacaagaatctgagcgagcgattggcaagccggtgtgtaaatggaagcgtgaggatgttgagagtaatttgacattcctagggttggtaggaatccgagacactcctcggatagagtccaaggaatctgtgagtcagtgtcaccaagctggtattactgtccacatgctcacgggagatcacaaagctacggctctgtccatcgccagggaagttggcatcttggagccactgtccgcatccaagagatctacatcgaaacgtggagtgaaaggtgatgcccatgtggtgcccatgtcatcgtctgtgatgacggcaacggagttcgacccgttgtcggagaaggaggtggacgcactggacgaacttccactggtgattgcgaggtgcacaccatcaacgaaggtgagaatgatcgacgcactgcacaggaggaagaagtatgcggctatgacgggagacggagtgaacgatgcgcccagtttgaagaaagcggacgtgggaatagccatgggagctgggagtgatgtggccaagacaagcagcgaaattgtacttacagacaacaatttcgcaacaattgttcaagccgtggccgagggacgtcgaatattttccaacatcaagaaattcgttgtgcatttgctgagcactaatgttggacaggtcattgtgctcctcggagggctggcgttcaaggatggctctggaatgtcagtgtttccactatcccctgtacagattctattcctgaatcttattacgggcacgccacccgccatggcattgggaattgagcgtgcctcctccacagtcatgcaagtacgaccatatctcaagggactattcacgaatgagctcatcgctgacatcctcgtttacggtaccttcaccggcgtcttggcccttctgaattgggtgcttgttatctacgcattcgggaatggcgaccttggcagtcagtgcaatacttcatcgaatctgggagcttgcgtgaccgtcttccgcgctagggcgactgtgcaaatggcattcacatggatgatcctatttcacgcttacaactgccgacacttgcgagcaagtctgttcaccacggaaggaggcggccgttctcgctttttcgccaacaaattgatggttgcctcggtgttcttgggcgctgtcgtgccagttcccaccctgtacataccggtcctcaacaccggcatcttcaagcaggaaggtctgacatgggagtggatccttgtcggtaccaccatggttgtgttctttctgttgtcggagttttacaagttgctcaagcggcgcttcatcaccaccccttacactatgtagactatgaattcattctgaactctaaccccaagctttggtttatcgtgtttacctattatcttaactgtagagaatacatacatatatatatatatatatatacatatctcgtggtgccgtacattcaatcaatggaataaacagaaataggccttagtagttgaagtttctgcgtcggtactgacgattggaatcgtaattcaatctaggaccagggtgaggaagtagccgaacgttgtagaaatttttctcgcctggtggtcagtttaacgattccaattttgtttaatgaattgatttcgtacaagttttttcactggattgaataacataatctgatcggtggcaagtttgctaattttagatcgtcagttccgttttgcggcaactattgattgggaagataaattgtggtgcagtggtttactgatacaatgctgggtatcgcatggcctcttcttagtcttcgtctcgatttggattgaagctcaacatcactcaaacactgcttggtcaagagtgataacaagagatgaagagttcctagtgcaggtgagcagtaactatgacatgctgaaaaccatgcaagtgaaagtaatacttgattgggcacttaagtgaaccagcgtagtttgtctcaatgtaaataggtaacaatctctgaggtgggatatgagacgttaggaaaggaattgatcaagcacttctgttgaattcatggtctacactcccaccttttgaccgtcgtagagagattcttgcagcatggaacttttggaatgtcctggagcatccctgatgaagatattccctcctccagttggcagagacgatcggggaagatgccgaaatgcgtgactgcaagagtgaagcggagaaaggttcgtgattaaaaaagacgatcacatttgcttggacactgatgtggtagaacaggcttaggtct

SEQ ID NO: 4 - DNA, 2814 bp, Physcomitrella patens
atggtcgacatccgagagttgatggaggtaccatgcgacgaatcatgctcaaaccatagcaagtccttcgaggaagttatcaaggttctagacagcaattcggagttggggctctcgaatgccaaagctgagcgattgctgaagcagtacggtcgcaacgagctcaagggtcagggggcagtgaacccatggaaaattttgcttgcgcaagtggccaacggcctgacggccgtgctgacaatcgctatggtggtgtctttcgccgtaaaagactacggcgaaggaggagtgttggtgctagtgattgcattcaacaccatcgttggattcatgcaagagtatcgcgcggagaaaaccatggatgccttgcgaaagatggcgtcgccgtcggcgaaggtcatccgagagggtattcagcaaagaatctccagtacggatgttgttccaggagatgtgctgactttcgaggtcggcgacattattcctgcagactgccgactgatggaggtcctgaatcttgaagttgacgaggctttgctcactggtgaatctgtaccttcgatcaagctcgtagagccaattctgggcaaggatgtctcgattggcgatcgcatcaacatggtgtactcgagcaccacggtggcgaagggaagaggtcgagcaatcgttgcatccaccgggatgtccactgagattggaaagatttccaacgcaatcaatgaaacacctgcgcaattgactctcttgcagcggaggcttaacatgatggcatacgtgctgatggtgatcgcgctcatcttagcactgattgtttttgctgtgaacaaatttgattttaacacggaaataattatctacgccatcgccctcgcaattggtgtcatcccagaggggcttatcgcggtcattaccattgtgcaagctctcggcgtacggcggatggccaagcagcatgcactggtgcggaagctggtagctttagagtccctgcaggcggtgacaaacatctgctcagacaagaccggcactttgacgcagggcaagatggtggtgacgaatgtttggttccctggacacgactccgagtacatcatctccggccagggatacgagaccaaaggcgacatttcggctcagggccgtgctatcgccacagagacagcgctcgaggacttgaatttccggaagctggtggagtgctgcgcgttatgcaacacggcaaacatcgttgaggcttcacagggcaacgtgtggggggatccaactgagattgctctgcaggtgttcgcgtacaaaatgcagatggggaagcctatcctgcgcaagaccaagaagccggttgaggagttccccttcagctccgacaccaagcggatgagcatggtttacctaacaaaactcgaagacgtgcttgaagtttattcaaagggggcagaggttgtgctgacaatctgcgacgatgtgatggaacggacggggaacctcaggaacatcagcgaggacggtgagttcatgaagaatgtgactgtacaacaagaggagatggcgaaacagggtttaagggttttagtgatagcctacagacaagaatctgagcgagcgattggcaagccggtgtgtaaatggaagcgtgaggatgttgagagtaatttgacattcctagggttggtaggaatccgagacactcctcggatagagtccaaggaatctgtgagtcagtgtcaccaagctggtattactgtccacatgctcacgggagatcacaaagctacggctctgtccatcgccagggaagttggcatcttggagccactgtccgcatccaagagatctacatcgaaacgtggagtgaaaggtgatgcccatgtggtgcccatgtcatcgtctgtgatgacggcaacggagttcgacccgttgtcggagaaggaggtggacgcactggacgaacttccactggtgattgcgaggtgcacaccatcaacgaaggtgagaatgatcgacgcactgcacaggaggaagaagtatgcggctatgacgggagacggagtgaacgatgcgcccagtttgaagaaagcggacgtgggaatagccatgggagctgggagtgatgtggccaagacaagcagcgaaattgtacttacagacaacaatttcgcaacaattgttcaagccgtggccgagggacgtcgaatattttccaacatcaagaaattcgttgtgcatttgctgagcactaatgttggacaggtcattgtgctcctcggagggctggcgttcaaggatggctctggaatgtcagtgtttccactatcccctgtacagattctattcctgaatcttattacgggcacgccacccgccatggcattgggaattgagcgtgcctcctccacagtcatgcaagtacgaccatatctcaagggactattcacgaatgagctcatcgctgacatcctcgtttacggtaccttcaccggcgtcttggcccttctgaattgggtgcttgttatctacgcattcgggaatggcgaccttggcagtcagtgcaatacttcatcgaatctgggagcttgcgtgaccgtcttccgcgctagggcgactgtgcaaatggcattcacatggatgatcctatttcacgcttacaactgccgacacttgcgagcaagtctgttcaccacggaaggaggcggccgttctcgctttttcgccaacaaattgatggttgcctcggtgttcttgggcgctgtcgtgccagttcccaccctgtacataccggtcctcaacaccggcatcttcaagcaggaagtggtttac

SEQ ID NO: 5 - protein, 938 aa, Physcomitrella patens
MVDIRELMEVPCDESCSNHSKSFEEVIKVLDSNSELGLSNAKAERLLKQYGRNELKGQGAVNPWKILLAQVANGLTAVLTIAMVVSFAVKDYGEGGVLVLVIAFNTIVGFMQEYRAEKTMDALRKMASPSAKVIREGIQQRISSTDVVPGDVLTFEVGDIIPADCRLMEVLNLEVDEALLTGESVPSIKLVEPILGKDVSIGDRINMVYSSTTVAKGRGRAIVASTGMSTEIGKISNAINETPAQLTLLQRRLNMMAYVLMVIALILALIVFAVNKFDFNTEIIIYAIALAIGVIPEGLIAVITIVQALGVRRMAKQHALVRKLVALESLQAVTNICSDKTGTLTQGKMVVTNVWFPGHDSEYIISGQGYETKGDISAQGRAIATETALEDLNFRKLVECCALCNTANIVEASQGNVWGDPTEIALQVFAYKMQMGKPILRKTKKPVEEFPFSSDTKRMSMVYLTKLEDVLEVYSKGAEVVLTICDDVMERTGNLRNISEDGEFMKNVTVQQEEMAKQGLRVLVIAYRQESERAIGKPVCKWKREDVESNLTFLGLVGIRDTPRIESKESVSQCHQAGITVHMLTGDHKATALSIAREVGILEPLSASKRSTSKRGVKGDAHVVPMSSSVMTATEFDPLSEKEVDALDELPLVIARCTPSTKVRMIDALHRRKKYAAMTGDGVNDAPSLKKADVGIAMGAGSDVAKTSSEIVLTDNNFATIVQAVAEGRRIFSNIKKFVVHLLSTNVGQVIVLLGGLAFKDGSGMSVFPLSPVQILFLNLITGTPPAMALGIERASSTVMQVRPYLKGLFTNELIADILVYGTFTGVLALLNWVLVIYAFGNGDLGSQCNTSSNLGACVTVFRARATVQMAFTWMILFHAYNCRHLRASLFTTEGGGRSRFFANKLMVASVFLGAVVPVPTLYIPVLNTGIFKQEVVY

SEQ ID NO: 6 - DNA, 3809 bp, Physcomitrella patens
atggtcgacatccgagagttgatggaggtaccatgcgacgaatcatgctcaaaccatagcaagtccttcgaggaagttatcaaggttctagacagcaattcggagttggggctctcgaatgccaaagctgagcgattgctgaagcagtacggtcgcaacgagctcaagggtcagggggcagtgaacccatggaaaattttgcttgcgcaagtggccaacggcctgacggccgtgctgacaatcgctatggtggtgtctttcgccgtaaaagactacggcgaaggaggagtgttggtgctagtgattgcattcaacaccatcgttggattcatgcaagagtatcgcgcggagaaaaccatggatgccttgcgaaagatggcgtcgccgtcggcgaaggtcatccgagagggtattcagcaaagaatctccagtacggatgttgttccaggagatgtgctgactttcgaggtcggcgacattattcctgcagactgccgactgatggaggtcctgaatcttgaagttgacgaggctttgctcactggtgaatctgtaccttcgatcaagctcgtagagccaattctgggcaaggatgtctcgattggcgatcgcatcaacatggtgtactcgagcaccacggtggcgaagggaagaggtcgagcaatcgttgcatccaccgggatgtccactgagattggaaagatttccaacgcaatcaatgaaacacctgcgcaattgactctcttgcagcggaggcttaacatgatggcatacgtgctgatggtgatcgcgctcatcttagcactgattgtttttgctgtgaacaaatttgattttaacacggaaataattatctacgccatcgccctcgcaattggtgtcatcccagaggggcttatcgcggtcattaccattgtgcaagctctcggcgtacggcggatggccaagcagcatgcactggtgcggaagctggtagctttagagtccctgcaggcggtgacaaacatctgctcagacaagaccggcactttgacgcagggcaagatggtggtgacgaatgtttggttccctggacacgactccgagtacatcatctccggccagggatacgagaccaaaggcgacatttcggctcagggccgtgctatcgccacagagacagcgctcgaggacttgaatttccggaagctggtggagtgctgcgcgttatgcaacacggcaaacatcgttgaggcttcacagggcaacgtgtggggggatccaactgagattgctctgcaggtgttcgcgtacaaaatgcagatggggaagcctatcctgcgcaagaccaagaagccggttgaggagttccccttcagctccgacaccaagcggatgagcatggtttacctaacaaaactcgaagacgtgcttgaagtttattcaaagggggcagaggttgtgctgacaatctgcgacgatgtgatggaacggacggggaacctcaggaacatcagcgaggacggtgagttcatgaagaatgtgactgtacaacaagaggagatggcgaaacagggtttaagggttttagtgatagcctacagacaagaatctgagcgagcgattggcaagccggtgtgtaaatggaagcgtgaggatgttgagagtaatttgacattcctagggttggtaggaatccgagacactcctcggatagagtccaaggaatctgtgagtcagtgtcaccaagctggtattactgtccacatgctcacgggagatcacaaagctacggctctgtccatcgccagggaagttggcatcttggagccactgtccgcatccaagagatctacatcgaaacgtggagtgaaaggtgatgcccatgtggtgcccatgtcatcgtctgtgatgacggcaacggagttcgacccgttgtcggagaaggaggtggacgcactggacgaacttccactggtgattgcgaggtgcacaccatcaacgaaggtgagaatgatcgacgcactgcacaggaggaagaagtatgcggctatgacgggagacggagtgaacgatgcgcccagtttgaagaaagcggacgtgggaatagccatgggagctgggagtgatgtggccaagacaagcagcgaaattgtacttacagacaacaatttcgcaacaattgttcaagccgtggccgagggacgtcgaatattttccaacatcaagaaattcgttgtgcatttgctgagcactaatgttggacaggtcattgtgctcctcggagggctggcgttcaaggatggctctggaatgtcagtgtttccactatcccctgtacagattctattcctgaatcttattacgggcacgccacccgccatggcattgggaattgagcgtgcctcctccacagtcatgcaagtacgaccatatctcaagggactattcacgaatgagctcatcgctgacatcctcgtttacggtaccttcaccggcgtcttggcccttctgaattgggtgcttgttatctacgcattcgggaatggcgaccttggcagtcagtgcaatacttcatcgaatctgggagcttgcgtgaccgtcttccgcgctagggcgactgtgcaaatggcattcacatggatgatcctatttcacgcttacaactgccgacacttgcgagcaagtctgttcaccacggaaggaggcggccgttctcgctttttcgccaacaaattgatggttgcctcggtgttcttgggcgctgtcgtgccagttcccaccctgtacataccggtcctcaacaccggcatcttcaagcaggaaggtctgacatgggagtggatccttgtcggtaccaccatggttgtgttctttctgttgtcggagttttacaagttgctcaagcggcgcttcatcaccaccccttacactatgtagactatgaattcattctgaactctaaccccaagctttggtttatcgtgtttacctattatcttaactgtagagaatacatacatatatatatatatatatatacatatctcgtggtgccgtacattcaatcaatggaataaacagaaataggccttagtagttgaagtttctgcgtcggtactgacgattggaatcgtaattcaatctaggaccagggtgaggaagtagccgaacgttgtagaaatttttctcgcctggtggtcagtttaacgattccaattttgtttaatgaattgatttcgtacaagttttttcactggattgaataacataatctgatcggtggcaagtttgctaattttagatcgtcagttccgttttgcggcaactattgattgggaagataaattgtggtgcagtggtttactgatacaatgctgggtatcgcatggcctcttcttagtcttcgtctcgatttggattgaagctcaacatcactcaaacactgcttggtcaagagtgataacaagagatgaagagttcctagtgcaggtgagcagtaactatgacatgctgaaaaccatgcaagtgaaagtaatacttgattgggcacttaagtgaaccagcgtagtttgtctcaatgtaaataggtaacaatctctgaggtgggatatgagacgttaggaaaggaattgatcaagcacttctgttgaattcatggtctacactcccaccttttgaccgtcgtagagagattcttgcagcatggaacttttggaatgtcctggagcatccctgatgaagatattccctcctccagttggcagagacgatcggggaagatgccgaaatgcgtgactgcaagagtgaagcggagaaaggttcgtgat

SEQ ID NO: 7 - DNA, 3174 bp, Physcomitrella patens
atggtcgacatccgagagttgatggaggtaccatgcgacgaatcatgctcaaaccatagcaagtccttcgaggaagttatcaaggttctagacagcaattcggagttggggctctcgaatgccaaagctgagcgattgctgaagcagtacggtcgcaacgagctcaagggtcagggggcagtgaacccatggaaaattttgcttgcgcaagtggccaacggcctgacggccgtgctgacaatcgctatggtggtgtctttcgccgtaaaagactacggcgaaggaggagtgttggtgctagtgattgcattcaacaccatcgttggattcatgcaagagtatcgcgcggagaaaaccatggatgccttgcgaaagatggcgtcgccgtcggcgaaggtcatccgagagggtattcagcaaagaatctccagtacggatgttgttccaggagatgtgctgactttcgaggtcggcgacattattcctgcagactgccgactgatggaggtcctgaatcttgaagttgacgaggctttgctcactggtgaatctgtaccttcgatcaagctcgtagagccaattctgggcaaggatgtctcgattggcgatcgcatcaacatggtgtactcgagcaccacggtggcgaagggaagaggtcgagcaatcgttgcatccaccgggatgtccactgagattggaaagatttccaacgcaatcaatgaaacacctgcgcaattgactctcttgcagcggaggcttaacatgatggcatacgtgctgatggtgatcgcgctcatcttagcactgattgtttttgctgtgaacaaatttgattttaacacggaaataattatctacgccatcgccctcgcaattggtgtcatcccagaggggcttatcgcggtcattaccattgtgcaagctctcggcgtacggcggatggccaagcagcatgcactggtgcggaagctggtagctttagagtccctgcaggcggtgacaaacatctgctcagacaagaccggcactttgacgcagggcaagatggtggtgacgaatgtttggttccctggacacgactccgagtacatcatctccggccagggatacgagaccaaaggcgacatttcggctcagggccgtgctatcgccacagagacagcgctcgaggacttgaatttccggaagctggtggagtgctgcgcgttatgcaacacggcaaacatcgttgaggcttcacagggcaacgtgtggggggatccaactgagattgctctgcaggtgttcgcgtacaaaatgcagatggggaagcctatcctgcgcaagaccaagaagccggttgaggagttccccttcagctccgacaccaagcggatgagcatggtttacctaacaaaactcgaagacgtgcttgaagtttattcaaagggggcagaggttgtgctgacaatctgcgacgatgtgatggaacggacggggaacctcaggaacatcagcgaggacggtgagttcatgaagaatgtgactgtacaacaagaggagatggcgaaacagggtttaagggttttagtgatagcctacagacaagaatctgagcgagcgattggcaagccggtgtgtaaatggaagcgtgaggatgttgagagtaatttgacattcctagggttggtaggaatccgagacactcctcggatagagtccaaggaatctgtgagtcagtgtcaccaagctggtattactgtccacatgctcacgggagatcacaaagctacggctctgtccatcgccagggaagttggcatcttggagccactgtccgcatccaagagatctacatcgaaacgtggagtgaaaggtgatgcccatgtggtgcccatgtcatcgtctgtgatgacggcaacggagttcgacccgttgtcggagaaggaggtggacgcactggacgaacttccactggtgattgcgaggtgcacaccatcaacgaaggtgagaatgatcgacgcactgcacaggaggaagaagtatgcggctatgacgggagacggagtgaacgatgcgcccagtttgaagaaagcggacgtgggaatagccatgggagctgggagtgatgtggccaagacaagcagcgaaattgtacttacagacaacaatttcgcaacaattgttcaagccgtggccgagggacgtcgaatattttccaacatcaagaaattcgttgtgcatttgctgagcactaatgttggacaggtcattgtgctcctcggagggctggcgttcaaggatggctctggaatgtcagtgtttccactatcccctgtacagattctattcctgaatcttattacgggcacgccacccgccatggcattgggaattgagcgtgcctcctccacagtcatgcaagtacgaccatatctcaagggactattcacgaatgagctcatcgctgacatcctcgtttacggtaccttcaccggcgtcttggcccttctgaattgggtgcttgttatctacgcattcgggaatggcgaccttggcagtcagtgcaatacttcatcgaatctgggagcttgcgtgaccgtcttccgcgctagggcgactgtgcaaatggcattcacatggatgatcctatttcacgcttacaactgccgacacttgcgagcaagtctgttcaccacggaaggaggcggccgttctcgctttttcgccaacaaattgatggttgcctcggtgttcttgggcgctgtcgtgccagttcccaccctgtacataccggtcctcaacaccggcatcttcaagcaggaaggtctgacatgggagtggatccttgtcggtaccaccatggttgtgttctttctgttgtcggagttttacaagttgctcaagcggcgcttcatcaccaccccttacactattggtttactgatacaatgctgggtatcgcatggcctcttcttagtcttcgtctcgatttggattgaagctcaacatcactcaaacactgcttggtcaagagtgataacaagagatgaagagttcctagtgcagcatggaacttttggaatgtcctggagcatccctgatgaagatattccctcctccagttggcagagacgatcggggaagatgccgaaatgcgtgactgcaagagtgaagcggagaaaggttcgtgat

SEQ ID NO: 8 - protein, 1058 aa, Physcomitrella patens
MVDIRELMEVPCDESCSNHSKSFEEVIKVLDSNSELGLSNAKAERLLKQYGRNELKGQGAVNPWKILLAQVANGLTAVLTIAMVVSFAVKDYGEGGVLVLVIAFNTIVGFMQEYRAEKTMDALRKMASPSAKVIREGIQQRISSTDVVPGDVLTFEVGDIIPADCRLMEVLNLEVDEALLTGESVPSIKLVEPILGKDVSIGDRINMVYSSTTVAKGRGRAIVASTGMSTEIGKISNAINETPAQLTLLQRRLNMMAYVLMVIALILALIVFAVNKFDFNTEIIIYAIALAIGVIPEGLIAVITIVQALGVRRMAKQHALVRKLVALESLQAVTNICSDKTGTLTQGKMVVTNVWFPGHDSEYIISGQGYETKGDISAQGRAIATETALEDLNFRKLVECCALCNTANIVEASQGNVWGDPTEIALQVFAYKMQMGKPILRKTKKPVEEFPFSSDTKRMSMVYLTKLEDVLEVYSKGAEVVLTICDDVMERTGNLRNISEDGEFMKNVTVQQEEMAKQGLRVLVIAYRQESERAIGKPVCKWKREDVESNLTFLGLVGIRDTPRIESKESVSQCHQAGITVHMLTGDHKATALSIAREVGILEPLSASKRSTSKRGVKGDAHVVPMSSSVMTATEFDPLSEKEVDALDELPLVIARCTPSTKVRMIDALHRRKKYAAMTGDGVNDAPSLKKADVGIAMGAGSDVAKTSSEIVLTDNNFATIVQAVAEGRRIFSNIKKFVVHLLSTNVGQVIVLLGGLAFKDGSGMSVFPLSPVQILFLNLITGTPPAMALGIERASSTVMQVRPYLKGLFTNELIADILVYGTFTGVLALLNWVLVIYAFGNGDLGSQCNTSSNLGACVTVFRARATVQMAFTWMILFHAYNCRHLRASLFTTEGGGRSRFFANKLMVASVFLGAVVPVPTLYIPVLNTGIFKQEGLTWEWILVGTTMVVFFLLSEFYKLLKRRFITTPYTIGLLIQCWVSHGLFLVFVSIWIEAQHHSNTAWSRVITRDEEFLVQHGTFGMSWSIPDEDIPSSSWQRRSGKMPKCVTARVKRRKVRD

SEQ ID NO: 9 - DNA, 3144 bp, Physcomitrella patens
atggtcgacatccgagagttgatggaggtaccatgcgacgaatcatgctcaaaccatagcaagtccttcgaggaagttatcaaggttctagacagcaattcggagttggggctctcgaatgccaaagctgagcgattgctgaagcagtacggtcgcaacgagctcaagggtcagggggcagtgaacccatggaaaattttgcttgcgcaagtggccaacggcctgacggccgtgctgacaatcgctatggtggtgtctttcgccgtaaaagactacggcgaaggaggagtgttggtgctagtgattgcattcaacaccatcgttggattcatgcaagagtatcgcgcggagaaaaccatggatgccttgcgaaagatggcgtcgccgtcggcgaaggtcatccgagagggtattcagcaaagaatctccagtacggatgttgttccaggagatgtgctgactttcgaggtcggcgacattattcctgcagactgccgactgatggaggtcctgaatcttgaagttgacgaggctttgctcactggtgaatctgtaccttcgatcaagctcgtagagccaattctgggcaaggatgtctcgattggcgatcgcatcaacatggtgtactcgagcaccacggtggcgaagggaagaggtcgagcaatcgttgcatccaccgggatgtccactgagattggaaagatttccaacgcaatcaatgaaacacctgcgcaattgactctcttgcagcggaggcttaacatgatggcatacgtgctgatggtgatcgcgctcatcttagcactgattgtttttgctgtgaacaaatttgattttaacacggaaataattatctacgccatcgccctcgcaattggtgtcatcccagaggggcttatcgcggtcattaccattgtgcaagctctcggcgtacggcggatggccaagcagcatgcactggtgcggaagctggtagctttagagtccctgcaggcggtgacaaacatctgctcagacaagaccggcactttgacgcagggcaagatggtggtgacgaatgtttggttccctggacacgactccgagtacatcatctccggccagggatacgagaccaaaggcgacatttcggctcagggccgtgctatcgccacagagacagcgctcgaggacttgaatttccggaagctggtggagtgctgcgcgttatgcaacacggcaaacatcgttgaggcttcacagggcaacgtgtggggggatccaactgagattgctctgcaggtgttcgcgtacaaaatgcagatggggaagcctatcctgcgcaagaccaagaagccggttgaggagttccccttcagctccgacaccaagcggatgagcatggtttacctaacaaaactcgaagacgtgcttgaagtttattcaaagggggcagaggttgtgctgacaatctgcgacgatgtgatggaacggacggggaacctcaggaacatcagcgaggacggtgagttcatgaagaatgtgactgtacaacaagaggagatggcgaaacagggtttaagggttttagtgatagcctacagacaagaatctgagcgagcgattggcaagccggtgtgtaaatggaagcgtgaggatgttgagagtaatttgacattcctagggttggtaggaatccgagacactcctcggatagagtccaaggaatctgtgagtcagtgtcaccaagctggtattactgtccacatgctcacgggagatcacaaagctacggctctgtccatcgccagggaagttggcatcttggagccactgtccgcatccaagagatctacatcgaaacgtggagtgaaaggtgatgcccatgtggtgcccatgtcatcgtctgtgatgacggcaacggagttcgacccgttgtcggagaaggaggtggacgcactggacgaacttccactggtgattgcgaggtgcacaccatcaacgaaggtgagaatgatcgacgcactgcacaggaggaagaagtatgcggctatgacgggagacggagtgaacgatgcgcccagtttgaagaaagcggacgtgggaatagccatgggagctgggagtgatgtggccaagacaagcagcgaaattgtacttacagacaacaatttcgcaacaattgttcaagccgtggccgagggacgtcgaatattttccaacatcaagaaattcgttgtgcatttgctgagcactaatgttggacaggtcattgtgctcctcggagggctggcgttcaaggatggctctggaatgtcagtgtttccactatcccctgtacagattctattcctgaatcttattacgggcacgccacccgccatggcattgggaattgagcgtgcctcctccacagtcatgcaagtacgaccatatctcaagggactattcacgaatgagctcatcgctgacatcctcgtttacggtaccttcaccggcgtcttggcccttctgaattgggtgcttgttatctacgcattcgggaatggcgaccttggcagtcagtgcaatacttcatcgaatctgggagcttgcgtgaccgtcttccgcgctagggcgactgtgcaaatggcattcacatggatgatcctatttcacgcttacaactgccgacacttgcgagcaagtctgttcaccacggaaggaggcggccgttctcgctttttcgccaacaaattgatggttgcctcggtgttcttgggcgctgtcgtgccagttcccaccctgtacataccggtcctcaacaccggcatcttcaagcaggaaggtctgacatgggagtggatccttgtcggtaccaccatggttgtgttctttctgttgtcggagttttacaagttgctcaagcggcgcttcatcaccaccccttacactatgtagactatgaattcattctgaactctaaccccaagctttggtttatcgtgtttacctattatcttaactgtagagaatacatacatatatatatatatatatatacatatctcgtggtgccgtacattcaatcaatggaataaacagaaataggccttagtagttgaagtttctgcgtcggtactgacgattggaatcgtaattcaatctaggaccagggtgaggaag

SEQ ID NO: 10 - DNA, 2931 bp, Physcomitrella patens
atggtcgacatccgagagttgatggaggtaccatgcgacgaatcatgctcaaaccatagcaagtccttcgaggaagttatcaaggttctagacagcaattcggagttggggctctcgaatgccaaagctgagcgattgctgaagcagtacggtcgcaacgagctcaagggtcagggggcagtgaacccatggaaaattttgcttgcgcaagtggccaacggcctgacggccgtgctgacaatcgctatggtggtgtctttcgccgtaaaagactacggcgaaggaggagtgttggtgctagtgattgcattcaacaccatcgttggattcatgcaagagtatcgcgcggagaaaaccatggatgccttgcgaaagatggcgtcgccgtcggcgaaggtcatccgagagggtattcagcaaagaatctccagtacggatgttgttccaggagatgtgctgactttcgaggtcggcgacattattcctgcagactgccgactgatggaggtcctgaatcttgaagttgacgaggctttgctcactggtgaatctgtaccttcgatcaagctcgtagagccaattctgggcaaggatgtctcgattggcgatcgcatcaacatggtgtactcgagcaccacggtggcgaagggaagaggtcgagcaatcgttgcatccaccgggatgtccactgagattggaaagatttccaacgcaatcaatgaaacacctgcgcaattgactctcttgcagcggaggcttaacatgatggcatacgtgctgatggtgatcgcgctcatcttagcactgattgtttttgctgtgaacaaatttgattttaacacggaaataattatctacgccatcgccctcgcaattggtgtcatcccagaggggcttatcgcggtcattaccattgtgcaagctctcggcgtacggcggatggccaagcagcatgcactggtgcggaagctggtagctttagagtccctgcaggcggtgacaaacatctgctcagacaagaccggcactttgacgcagggcaagatggtggtgacgaatgtttggttccctggacacgactccgagtacatcatctccggccagggatacgagaccaaaggcgacatttcggctcagggccgtgctatcgccacagagacagcgctcgaggacttgaatttccggaagctggtggagtgctgcgcgttatgcaacacggcaaacatcgttgaggcttcacagggcaacgtgtggggggatccaactgagattgctctgcaggtgttcgcgtacaaaatgcagatggggaagcctatcctgcgcaagaccaagaagccggttgaggagttccccttcagctccgacaccaagcggatgagcatggtttacctaacaaaactcgaagacgtgcttgaagtttattcaaagggggcagaggttgtgctgacaatctgcgacgatgtgatggaacggacggggaacctcaggaacatcagcgaggacggtgagttcatgaagaatgtgactgtacaacaagaggagatggcgaaacagggtttaagggttttagtgatagcctacagacaagaatctgagcgagcgattggcaagccggtgtgtaaatggaagcgtgaggatgttgagagtaatttgacattcctagggttggtaggaatccgagacactcctcggatagagtccaaggaatctgtgagtcagtgtcaccaagctggtattactgtccacatgctcacgggagatcacaaagctacggctctgtccatcgccagggaagttggcatcttggagccactgtccgcatccaagagatctacatcgaaacgtggagtgaaaggtgatgcccatgtggtgcccatgtcatcgtctgtgatgacggcaacggagttcgacccgttgtcggagaaggaggtggacgcactggacgaacttccactggtgattgcgaggtgcacaccatcaacgaaggtgagaatgatcgacgcactgcacaggaggaagaagtatgcggctatgacgggagacggagtgaacgatgcgcccagtttgaagaaagcggacgtgggaatagccatgggagctgggagtgatgtggccaagacaagcagcgaaattgtacttacagacaacaatttcgcaacaattgttcaagccgtggccgagggacgtcgaatattttccaacatcaagaaattcgttgtgcatttgctgagcactaatgttggacaggtcattgtgctcctcggagggctggcgttcaaggatggctctggaatgtcagtgtttccactatcccctgtacagattctattcctgaatcttattacgggcacgccacccgccatggcattgggaattgagcgtgcctcctccacagtcatgcaagtacgaccatatctcaagggactattcacgaatgagctcatcgctgacatcctcgtttacggtaccttcaccggcgtcttggcccttctgaattgggtgcttgttatctacgcattcgggaatggcgaccttggcagtcagtgcaatacttcatcgaatctgggagcttgcgtgaccgtcttccgcgctagggcgactgtgcaaatggcattcacatggatgatcctatttcacgcttacaactgccgacacttgcgagcaagtctgttcaccacggaaggaggcggccgttctcgctttttcgccaacaaattgatggttgcctcggtgttcttgggcgctgtcgtgccagttcccaccctgtacataccggtcctcaacaccggcatcttcaagcaggaaggtctgacatgggagtggatccttgtcggtaccaccatggttgtgttctttctgttgtcggagttttacaagttgctcaagcggcgcttcatcaccaccccttacactatgaccagggtgaggaag

SEQ ID NO: 11 - protein, 977 aa, Physcomitrella patens
MVDIRELMEVPCDESCSNHSKSFEEVIKVLDSNSELGLSNAKAERLLKQYGRNELKGQGAVNPWKILLAQVANGLTAVLTIAMVVSFAVKDYGEGGVLVLVIAFNTIVGFMQEYRAEKTMDALRKMASPSAKVIREGIQQRISSTDVVPGDVLTFEVGDIIPADCRLMEVLNLEVDEALLTGESVPSIKLVEPILGKDVSIGDRINMVYSSTTVAKGRGRAIVASTGMSTEIGKISNAINETPAQLTLLQRRLNMMAYVLMVIALILALIVFAVNKFDFNTEIIIYAIALAIGVIPEGLIAVITIVQALGVRRMAKQHALVRKLVALESLQAVTNICSDKTGTLTQGKMVVTNVWFPGHDSEYIISGQGYETKGDISAQGRAIATETALEDLNFRKLVECCALCNTANIVEASQGNVWGDPTEIALQVFAYKMQMGKPILRKTKKPVEEFPFSSDTKRMSMVYLTKLEDVLEVYSKGAEVVLTICDDVMERTGNLRNISEDGEFMKNVTVQQEEMAKQGLRVLVIAYRQESERAIGKPVCKWKREDVESNLTFLGLVGIRDTPRIESKESVSQCHQAGITVHMLTGDHKATALSIAREVGILEPLSASKRSTSKRGVKGDAHVVPMSSSVMTATEFDPLSEKEVDALDELPLVIARCTPSTKVRMIDALHRRKKYAAMTGDGVNDAPSLKKADVGIAMGAGSDVAKTSSEIVLTDNNFATIVQAVAEGRRIFSNIKKFVVHLLSTNVGQVIVLLGGLAFKDGSGMSVFPLSPVQILFLNLITGTPPAMALGIERASSTVMQVRPYLKGLFTNELIADILVYGTFTGVLALLNWVLVIYAFGNGDLGSQCNTSSNLGACVTVFRARATVQMAFTWMILFHAYNCRHLRASLFTTEGGGRSRFFANKLMVASVFLGAVVPVPTLYIPVLNTGIFKQEGLTWEWILVGTTMVVFFLLSEFYKLLKRRFITTPYTMTRVRK

SEQ ID NO: 12 - DNA, 3276 bp, Saccharomyces cerevisiae
atgggcgaaggaactactaaggaaaacaataatgcagaattcaatgcttatcacacgctgactgcagaagaagccgctgaattcataggcacaagtctaactgaaggtttgacccaagatgagttcgtccacagattgaaaacagtgggtgagaacacattgggtgatgacactaaaattgattacaaggcaatggtcctccatcaggtatgtaatgccatgatcatggtccttctaatatccatgataatctcgtttgccatgcatgattggattactggtggcgttatttcttttgttatcgcggtcaatgtgctcattggcctagttcaagaatataaggctaccaagacaatgaactctttgaaaaacttgagctctcccaatgctcatgttattaggaacgggaaaagtgagactataaactcaaaagatgtggttccaggtgatatttgtctggtgaaggtcggtgatactattcctgctgatttgcgtttaattgaaactaagaattttgatactgatgaatcactactgaccggtgaatctttgcctgtctccaaagacgccaacttagtgtttggaaaagaagaagaaacctccgtgggtgatcgtttgaatttagcattttcttcatccgccgttgtcaagggaagagccaagggtattgtcatcaagacagctttgaatagtgaaattggtaaaattgcaaaatctctacaaggtgattcaggtctcatttcccgcgatcctagtaagtcgtggttacagaatacatggatatctacaaagaaagttactggtgcatttttaggtacaaatgtcggtacgcccctgcacaggaaactgtctaagttggcggtattgcttttctggattgccgtactttttgctatcatcgtcatggcctctcaaaagtttgacgtagataaaagggtagctatctatgccatttgtgtggccctatccatgattccctcttcattagtcgttgtcttgaccatcaccatgtctgttggggctgctgttatggtttctagaaacgttattgtaagaaaattagattctctggaagctttaggtgccgttaacgatatctgttctgacaagaccggtactcttacacagggtaaaatgttagcgaggcaaatttggatccctcgctttggtaccataactatctcgaattctgatgacccctttaatcccaatgagggcaacgtgagtttgattccaaggttttcaccttacgaatattctcataatgaggatggtgacgttggtattctccagaatttcaaggatcgcctatacgaaaaagatttaccagaagatattgacatggatctatttcaaaaatggctcgaaaccgccactttggctaacattgctactgttttcaaagatgacgcaactgactgttggaaagctcatggtgacccaacagaaattgcgattcaagtgtttgctactaagatggacttgcctcacaatgcccttaccggtgagaaatcgactaatcaaagtaatgagaatgaccaatcctctctttcacaacacaatgagaagcctggcagtgcacaattcgaacatattgctgaattcccattcgactcaactgtgaagcgaatgtcatctgtctactacaacaatcacaacgaaacatataatatttatggcaagggtgctttcgaaagcatcatcagttgttgcagttcttggtatggtaaggatggtgtaaaaatcacaccattgaccgattgtgatgtcgaaacgataaggaaaaatgtttacagtctatcaaatgagggtttaagagtcttgggttttgcctccaaatctttcactaaagatcaagtgaatgacgatcaattgaaaaacattacttcaaacagggccaccgcagaaagtgatttagttttcctagggttgattggtatttacgatccacccagaaatgagactgccggtgcagtcaagaagtttcaccaagctggtattaacgttcatatgttaactggggactttgtgggtacagcaaaggctatcgctcaggaggttggcatcttacccaccaatttgtaccattactcccaagagattgttgacagtatggtcatgaccggatctcagtttgacggactaagtgaggaggaagtggacgatttgcccgtcttacctttagttattgcacgttgctctccgcagactaaggtgagaatgatcgaagctttacaccgtaggaagaagttctgcacaatgacaggtgacggtgttaacgattctccatctctaaaaatggccaatgttggtattgcaatgggtattaatggttcagatgtttccaaagaagcgtctgatattgttctaagcgatgacaattttgcttctattttgaatgctgtcgaagaaggtcgtaggatgacggataacattcagaagtttgtcctacaattattggcagaaaatgttgctcaggctttgtatttgatcattggtttagtattcagagatgagaacggaaaatcagtatttcccttatcaccagtggaagtattgtggattattgtcgtcacctcttgttttcctgctatggggctaggtctagaaaaggctgctccagatttgatggatagacctcctcatgattcagaggttggtattttcacgtgggaggttattatagatacatttgcatatgggattataatgacagggtcctgtatggcttcatttactggatcactgtatggaataaatagtggtagattggggcacgattgtgatggcacctataacagcagttgtcgtgatgtttatagatcacgttctgcggctttcgcaaccatgacgtggtgcgctttgattctggcttgggaagtggttgacatgagaagatcctttttcagaatgcatccagacactgacagcccagtcaaggaatttttcagaagcatttggggaaaccagtttttgttctggtcaatcatttttggatttgtgtcagccttccccgtcgtctatattccggttattaatgataaagtgtttttgcataaaccaattggtgctgaatggggtctcgccattgcattcacaattgcattctggataggtgctgaactttacaagtgtggaaagaggcgctatttcaaaactcagagagcgcacaacccggagaatgatttggagagtaacaataagcgcgatccattcgaagcgtatagtacttctactacaatccatacagaagttaatattggtattaaacaatga

SEQ ID NO: 13 - protein, 1091 aa, Saccharomyces cerevisiae
MGEGTTKENNNAEFNAYHTLTAEEAAEFIGTSLTEGLTQDEFVHRLKTVGENTLGDDTKIDYKAMVLHQVCNAMIMVLLISMIISFAMHDWITGGVISFVIAVNVLIGLVQEYKATKTMNSLKNLSSPNAHVIRNGKSETINSKDVVPGDICLVKVGDTIPADLRLIETKNFDTDESLLTGESLPVSKDANLVFGKEEETSVGDRLNLAFSSSAVVKGRAKGIVIKTALNSEIGKIAKSLQGDSGLISRDPSKSWLQNTWISTKKVTGAFLGTNVGTPLHRKLSKLAVLLFWIAVLFAIIVMASQKFDVDKRVAIYAICVALSMIPSSLVVVLTITMSVGAAVMVSRNVIVRKLDSLEALGAVNDICSDKTGTLTQGKMLARQIWIPRFGTITISNSDDPFNPNEGNVSLIPRFSPYEYSHNEDGDVGILQNFKDRLYEKDLPEDIDMDLFQKWLETATLANIATVFKDDATDCWKAHGDPTEIAIQVFATKMDLPHNALTGEKSTNQSNENDQSSLSQHNEKPGSAQFEHIAEFPFDSTVKRMSSVYYNNHNETYNIYGKGAFESIISCCSSWYGKDGVKITPLTDCDVETIRKNVYSLSNEGLRVLGFASKSFTKDQVNDDQLKNITSNRATAESDLVFLGLIGIYDPPRNETAGAVKKFHQAGINVHMLTGDFVGTAKAIAQEVGILPTNLYHYSQEIVDSMVMTGSQFDGLSEEEVDDLPVLPLVIARCSPQTKVRMIEALHRRKKFCTMTGDGVNDSPSLKMANVGIAMGINGSDVSKEASDIVLSDDNFASILNAVEEGRRMTDNIQKFVLQLLAENVAQALYLIIGLVFRDENGKSVFPLSPVEVLWIIVVTSCFPAMGLGLEKAAPDLMDRPPHDSEVGIFTWEVIIDTFAYGIIMTGSCMASFTGSLYGINSGRLGHDCDGTYNSSCRDVYRSRSAAFATMTWCALILAWEVVDMRRSFFRMHPDTDSPVKEFFRSIWGNQFLFWSIIFGFVSAFPVVYIPVINDKVFLHKPIGAEWGLAIAFTIAFWIGAELYKCGKRRYFKTQRAHNPENDLESNNKRDPFEAYSTSTTIHTEVNIGIKQ

SEQ ID NO: 14 - DNA, 2804 bp, Saccharomyces cerevisiae
atgagcgagggaactgtcaaagaaaacaataatgaagaattcaatgcttatcacacattgactacagaagaagccgctgaatttataggcacaagtctgactgaaggtcttactcaagatgagtccctccgcagactgaaagcagtgggagagaacacattgggtgatgataccaaaattgactataaagcaatggtcctccatcaggtatgtaatgcaatgattatggtcttggtaatatccatggcgatctctttcgctgtgcgcgactggattactggtggcgttatttcttttgttatcgcggtcaatgtgctcattggcctagttcaagaatataaggctaccaagacaatgaactctttgaaaaacttgagctctcccaatgctcatgttattaggaacgggaaaagtgagactataaactcaaaagatgtggttccaggtgatatttgtctggtgaaggtcggtgatactattcctgctgatttgcgtttaattgaaactaagaattttgatactgatgaatcactactgaccggtgaatctttgcctgtctccaaagacgccaacttagtgtttggaaaagaagaagaaacctccgtgggtgatcgtttgaatttagcattttcttcatccgccgttgtcaagggaagagccaagggtattgtcatcaagacagctttgaatagtgaaattggtaaaattgcaaaatctctacaaggtgattcaggtctcatttcccgcgatcctagtaagtcgtggttacagaatacatggatatctacaaagaaagttactggtgcatttttaggtacaaatgtcggtacgcccctgcacaggaaactgtctaagttggcggtattgcttttctggattgccgtactttttgctatcatcgtcatggcctctcaaaagtttgacgtagataaaagggtagctatctatgccatttgtgtggccctatccatgattccctcttcattagtcgttgtcttgaccatcaccatgtctgttggggctgctgttatggtttctagaaacgttattgtaagaaaattagattctctggaagctttaggtgccgttaacgatatctgttctgacaagaccggtactcttacacagggtaaaatgttagcgaggcaaatttggatccctcgctttggtaccataactatctcgaattctgatgacccctttaatcccaatgagggcaacgtgagtttgattccaaggttttcaccttacgaatattctcataatgaggatggtgacgttggtattctccagaatttcaaggatcgcctatacgaaaaagatttaccagaagatattgacatggatctatttcaaaaatggctcgaaaccgccactttggctaacattgctactgttttcaaagatgacgcaactgactgttggaaagctcatggtgacccaacagaaattgcgattcaagtgtttgctactaagatggacttgcctcacaatgcccttaccggtgagaaatcgactaatcaaagtaatgagaatgaccaatcctctctttcacaacacaatgagaagcctggcagtgcacaattcgaacatattgctgaattcccattcgactcaactgtgaagcgaatgtcatctgtctactacaacaatcacaacgaaacatataatatttatggcaagggtgctttcgaaagcatcatcagttgttgcagttcttggtatggtaaggatggtgtaaaaatcacaccattgaccgattgtgatgtcgaaacgataaggaaaaatgtttacagtctatcaaatgagggtttaagagtcttgggttttgcctccaaatctttcactaaagatcaagtgaatgacgatcaattgaaaaacattacttcaaacagggccaccgcagaaagtgatttagttttcctagggttgattggtatttacgatccacccagaaatgagactgccggtgcagtcaagaagtttcaccaagctggtattaacgttcatatgttaactggggactttgtgggtacagcaaaggctatcgctcaggaggttggcatcttacccaccaatttgtaccattactcccaagagattgttgacagtatggtcatgaccggatctcagtttgacggactaagtgaggaggaagtggacgatttgcccgtcttacctttagttattgcacgttgctctccgcagactaaggtgagaatgatcgaagctttacaccgtaggaagaagttctgcgcaatgacaggtgacggtgttaacgattctccatctctaaaaatggccaatgttggtattgcaatgggtattaatggttcagatgtttccaaagaagcgtctgatattgttctaagcgatgacaattttgcttctattttgaatgctgtcgaagaaggtcgtaggatgacggataacattcagaagtttgtcctacaattattggcagaaaatgttgctcaggctttgtatttgatcattggtttagtattcagagatgagaacggaaaatcagtatttcccttatcaccagtggaagtattgtggattattgtcgtcacctcttgttttcctgctatggggctaggtctagaaaaggctgctccagatttgatggatagacctcctaatgattcagaggttggtattttcacatgggaggttattatagatacatttgcatatgggattataatgacagggtcctgtatggcttcatttactggatcactgtatggaataaatagtggtagattggggcacgattgtgatggcacctataacagcag

SEQ ID NO: 15 - protein, 934 aa, Saccharomyces cerevisiae
MSEGTVKENNNEEFNAYHTLTTEEAAEFIGTSLTEGLTQDESLRRLKAVGENTLGDDTKIDYKAMVLHQVCNAMIMVLVISMAISFAVRDWITGGVISFVIAVNVLIGLVQEYKATKTMNSLKNLSSPNAHVIRNGKSETINSKDVVPGDICLVKVGDTIPADLRLIETKNFDTDESLLTGESLPVSKDANLVFGKEEETSVGDRLNLAFSSSAVVKGRAKGIVIKTALNSEIGKIAKSLQGDSGLISRDPSKSWLQNTWISTKKVTGAFLGTNVGTPLHRKLSKLAVLLFWIAVLFAIIVMASQKFDVDKRVAIYAICVALSMIPSSLVVVLTITMSVGAAVMVSRNVIVRKLDSLEALGAVNDICSDKTGTLTQGKMLARQIWIPRFGTITISNSDDPFNPNEGNVSLIPRFSPYEYSHNEDGDVGILQNFKDRLYEKDLPEDIDMDLFQKWLETATLANIATVFKDDATDCWKAHGDPTEIAIQVFATKMDLPHNALTGEKSTNQSNENDQSSLSQHNEKPGSAQFEHIAEFPFDSTVKRMSSVYYNNHNETYNIYGKGAFESIISCCSSWYGKDGVKITPLTDCDVETIRKNVYSLSNEGLRVLGFASKSFTKDQVNDDQLKNITSNRATAESDLVFLGLIGIYDPPRNETAGAVKKFHQAGINVHMLTGDFVGTAKAIAQEVGILPTNLYHYSQEIVDSMVMTGSQFDGLSEEEVDDLPVLPLVIARCSPQTKVRMIEALHRRKKFCAMTGDGVNDSPSLKMANVGIAMGINGSDVSKEASDIVLSDDNFASILNAVEEGRRMTDNIQKFVLQLLAENVAQALYLIIGLVFRDENGKSVFPLSPVEVLWIIVVTSCFPAMGLGLEKAAPDLMDRPPNDSEVGIFTWEVIIDTFAYGIIMTGSCMASFTGSLYGINSGRLGHDCDGTYNS

SEQ ID NO: 16 - DNA, 3276 bp, Saccharomyces cerevisiae
atgagcgagggaactgtcaaagaaaacaataatgaagaattcaatgcttatcacacattgactacagaagaagccgctgaatttataggcacaagtctgactgaaggtcttactcaagatgagtccctccgcagactgaaagcagtgggagagaacacattgggtgatgataccaaaattgactataaagcaatggtcctccatcaggtatgtaatgcaatgattatggtcttggtaatatccatggcgatctctttcgctgtgcgcgactggattactggtggcgttatttcttttgttatcgcggtcaatgtgctcattggcctagttcaagaatataaggctaccaagacaatgaactctttgaaaaacttgagctctcccaatgctcatgttattaggaacgggaaaagtgagactataaactcaaaagatgtggttccaggtgatatttgtctggtgaaggtcggtgatactattcctgctgatttgcgtttaattgaaactaagaattttgatactgatgaatcactactgaccggtgaatctttgcctgtctccaaagacgccaacttagtgtttggaaaagaagaagaaacctccgtgggtgatcgtttgaatttagcattttcttcatccgccgttgtcaagggaagagccaagggtattgtcatcaagacagctttgaatagtgaaattggtaaaattgcaaaatctctacaaggtgattcaggtctcatttcccgcgatcctagtaagtcgtggttacagaatacatggatatctacaaagaaagttactggtgcatttttaggtacaaatgtcggtacgcccctgcacaggaaactgtctaagttggcggtattgcttttctggattgccgtactttttgctatcatcgtcatggcctctcaaaagtttgacgtagataaaagggtagctatctatgccatttgtgtggccctatccatgattccctcttcattagtcgttgtcttgaccatcaccatgtctgttggggctgctgttatggtttctagaaacgttattgtaagaaaattagattctctggaagctttaggtgccgttaacgatatctgttctgacaagaccggtactcttacacagggtaaaatgttagcgaggcaaatttggatccctcgctttggtaccataactatctcgaattctgatgacccctttaatcccaatgagggcaacgtgagtttgattccaaggttttcaccttacgaatattctcataatgaggatggtgacgttggtattctccagaatttcaaggatcgcctatacgaaaaagatttaccagaagatattgacatggatctatttcaaaaatggctcgaaaccgccactttggctaacattgctactgttttcaaagatgacgcaactgactgttggaaagctcatggtgacccaacagaaattgcgattcaagtgtttgctactaagatggacttgcctcacaatgcccttaccggtgagaaatcgactaatcaaagtaatgagaatgaccaatcctctctttcacaacacaatgagaagcctggcagtgcacaattcgaacatattgctgaattcccattcgactcaactgtgaagcgaatgtcatctgtctactacaacaatcacaacgaaacatataatatttatggcaagggtgctttcgaaagcatcatcagttgttgcagttcttggtatggtaaggatggtgtaaaaatcacaccattgaccgattgtgatgtcgaaacgataaggaaaaatgtttacagtctatcaaatgagggtttaagagtcttgggttttgcctccaaatctttcactaaagatcaagtgaatgacgatcaattgaaaaacattacttcaaacagggccaccgcagaaagtgatttagttttcctagggttgattggtatttacgatccacccagaaatgagactgccggtgcagtcaagaagtttcaccaagctggtattaacgttcatatgttaactggggactttgtgggtacagcaaaggctatcgctcaggaggttggcatcttacccaccaatttgtaccattactcccaagagattgttgacagtatggtcatgaccggatctcagtttgacggactaagtgaggaggaagtggacgatttgcccgtcttacctttagttattgcacgttgctctccgcagactaaggtgagaatgatcgaagctttacaccgtaggaagaagttctgcgcaatgacaggtgacggtgttaacgattctccatctctaaaaatggccaatgttggtattgcaatgggtattaatggttcagatgtttccaaagaagcgtctgatattgttctaagcgatgacaattttgcttctattttgaatgctgtcgaagaaggtcgtaggatgacggataacattcagaagtttgtcctacaattattggcagaaaatgttgctcaggctttgtatttgatcattggtttagtattcagagatgagaacggaaaatcagtatttcccttatcaccagtggaagtattgtggattattgtcgtcacctcttgttttcctgctatggggctaggtctagaaaaggctgctccagatttgatggatagacctcctaatgattcagaggttggtattttcacatgggaggttattatagatacatttgcatatgggattataatgacagggtcctgtatggcttcatttactggatcactgtatggaataaatagtggtagattggggcacgattgtgatggcacctataacagcagttgtcgtgatgtttatagatcacgttctgcggctttcgcaactatgacgtggtgcgctttgattctggcttgggaagtggttgacatgagaagatcttttttcagaatgcatccagacactgacagcccagtcaaggaatttttcagaagtatttggggaaaccagtttttgttctggtcaatcatttttggatttgtgtcagccttccccgtcgtctatattccggttattaatgataaagtgtttttgcataaaccaattggtgctgaatggggtctcgccattgcattcacaattgcattctggataggtgctgaactttacaagtgtggaaagaggcgctatttcaaaactcagagagctcacaattcggaaaacgatttggagcgaagcagtaaacacgatccatttgaagcgtatagcacatccacaacccttcaaagcgaaattaatatcagtgtcaagcattaa

SEQ ID NO: 17 - protein, 1091 aa, Saccharomyces cerevisiae
MSEGTVKENNNEEFNAYHTLTTEEAAEFIGTSLTEGLTQDESLRRLKAVGENTLGDDTKIDYKAMVLHQVCNAMIMVLVISMAISFAVRDWITGGVISFVIAVNVLIGLVQEYKATKTMNSLKNLSSPNAHVIRNGKSETINSKDVVPGDICLVKVGDTIPADLRLIETKNFDTDESLLTGESLPVSKDANLVFGKEEETSVGDRLNLAFSSSAVVKGRAKGIVIKTALNSEIGKIAKSLQGDSGLISRDPSKSWLQNTWISTKKVTGAFLGTNVGTPLHRKLSKLAVLLFWIAVLFAIIVMASQKFDVDKRVAIYAICVALSMIPSSLVVVLTITMSVGAAVMVSRNVIVRKLDSLEALGAVNDICSDKTGTLTQGKMLARQIWIPRFGTITISNSDDPFNPNEGNVSLIPRFSPYEYSHNEDGDVGILQNFKDRLYEKDLPEDIDMDLFQKWLETATLANIATVFKDDATDCWKAHGDPTEIAIQVFATKMDLPHNALTGEKSTNQSNENDQSSLSQHNEKPGSAQFEHIAEFPFDSTVKRMSSVYYNNHNETYNIYGKGAFESIISCCSSWYGKDGVKITPLTDCDVETIRKNVYSLSNEGLRVLGFASKSFTKDQVNDDQLKNITSNRATAESDLVFLGLIGIYDPPRNETAGAVKKFHQAGINVHMLTGDFVGTAKAIAQEVGILPTNLYHYSQEIVDSMVMTGSQFDGLSEEEVDDLPVLPLVIARCSPQTKVRMIEALHRRKKFCAMTGDGVNDSPSLKMANVGIAMGINGSDVSKEASDIVLSDDNFASILNAVEEGRRMTDNIQKFVLQLLAENVAQALYLIIGLVFRDENGKSVFPLSPVEVLWIIVVTSCFPAMGLGLEKAAPDLMDRPPNDSEVGIFTWEVIIDTFAYGIIMTGSCMASFTGSLYGINSGRLGHDCDGTYNSSCRDVYRSRSAAFATMTWCALILAWEVVDMRRSFFRMHPDTDSPVKEFFRSIWGNQFLFWSIIFGFVSAFPVVYIPVINDKVFLHKPIGAEWGLAIAFTIAFWIGAELYKCGKRRYFKTQRAHNSENDLERSSKHDPFEAYSTSTTLQSEINISVKH

SEQ ID NO: 18 - DNA, 22 bp, Artificial Sequence
  Synthetic primer
atggtcgacatccgagagttga

SEQ ID NO: 19 - DNA, 19 bp, Artificial Sequence
  Synthetic primer
cagggtgggaactggcacg

SEQ ID NO: 20 - DNA, 22 bp, Artificial Sequence
  Synthetic primer
tcctcctctgctaacgtaagcc

SEQ ID NO: 21 - DNA, 23 bp, Artificial Sequence
  Synthetic primer
ctgtggttggagaagctagaacc

SEQ ID NO: 22 - DNA, 21 bp, Artificial Sequence
  Synthetic primer
tttttcggtgtaagctgagtg

SEQ ID NO: 23 - DNA, 20 bp, Artificial Sequence
  Synthetic primer
atcgaaaaacggagttggtg

SEQ ID NO: 24 - DNA, 35 bp, Artificial Sequence
  Synthetic primer
aaggcgcgcctctgtcataggacactacaatcaaa

SEQ ID NO: 25 - DNA, 31 bp, Artificial Sequence
  Synthetic primer
aaacgcgttgtttgtgaagactgaagagacg

SEQ ID NO: 26 - DNA, 22 bp, Artificial Sequence
  Synthetic primer
tgcttaccacagattgtgttcc

SEQ ID NO: 27 - DNA, 20 bp, Artificial Sequence
  Synthetic primer
aaggaagccgaagaaaggag

SEQ ID NO: 28 - DNA, 32 bp, Artificial Sequence
  Synthetic primer
aaggcgcgcctccaaatcataagcagttccat

SEQ ID NO: 29 - DNA, 28 bp, Artificial Sequence
  Synthetic primer
aaacgcgtctcaggcgattctggatctt

SEQ ID NO: 30 - DNA, 52 bp, Artificial Sequence
  Synthetic primer
ggggacaagtttgtacaaaaaagcaggcttgatggagggctctggggacaag

SEQ ID NO: 31 - DNA, 55 bp, Artificial Sequence
  Synthetic primer
ggggaccactttgtacaagaaagctgggtatcacatgttgtagggagttttaatg

SEQ ID NO: 32 - DNA, 53 bp, Artificial Sequence
  Synthetic primer
ggggacaagtttgtacaaaaaagcaggcttatgggcgaaggaactactaagga

SEQ ID NO: 33 - DNA, 56 bp, Artificial Sequence
  Synthetic primer
ggggaccactttgtacaagaaagctgggtttcattgtttaataccaatattaactt

SEQ ID NO: 34 - DNA, 20 bp, Artificial Sequence
  Synthetic primer
tcgtgactggggaagggaag

SEQ ID NO: 35 - DNA, 20 bp, Artificial Sequence
  Synthetic primer
acagcatgggtgcggattct

SEQ ID NO: 36 - DNA, 22 bp, Artificial Sequence
  Synthetic primer
atgggcgaaggaactactaagg

SEQ ID NO: 37 - DNA, 32 bp, Artificial Sequence
  Synthetic primer
attgtttaataccaatattaacttctgtatgg

SEQ ID NO: 38 - DNA, 20 bp, Artificial Sequence
  Synthetic primer
cctacatgctcctcgcattt

SEQ ID NO: 39 - DNA, 25 bp, Artificial Sequence
  Synthetic primer
tcacggtgttcccgtgacgagattc

SEQ ID NO: 40 - DNA, 286 bp, Arabidopsis thaliana
tcctcctctgctaacgtaagcctctctgttttttttctctgtttcttttgaaatgaatccaattagtgatgataatctgtgtttgatgtatcattgatttaacatcttgacaatgaatcgtgatcggaagtgataaagttatgggtcaacggtttcaaagagagagaaagacttttagagtcaactctcgactctttcttaattatgttattgctatttgtctcttttcttgaagtctgaacaattcttgggattgttttgcaggttctagcttctccaaccacag

SEQ ID NO: 41 - DNA, 2155 bp, Arabidopsis thaliana
tttttcggtgtaagctgagtgttaaatctgtcataggacactacaatcaaattataactctatatatactccatcgactatatattgactcaacatatcatgatagaattacatatgagtcagatgtaattttatgatctgttattgtggattcattgttagcaagcatatatatacgtgtgttcagaagactaaaaaatttactatgaaatgaaagaacatttatttgtttggaacaaaaaatgtttatcataacaaattaacccatttttccacagagaacaataccattcgtaaccaatatcgagtagacaattctttaacaaaacagaaagcgacaaagatgaaagaaaacaaaaataaaaccaggggagagaccgagagagagagagcacctttctgacgtggaaatataatccaataagagcagagaaatgtcgctacaacgataaggatattgtgacgtggcaaaacattggccatgaacaccacattacaccacattccttttgtctatgtacactttttatttttccaatttatttttgtagacagactagtgattagtgaatactgaataatatacgtaagaaaattgcaattggaaatttggaattgaggagtgagagcaaatgattgttgaatatggggactaaacaacgtggcataagaggagtggttgggacggctgaactggagttggacttaatctgtatggacggtgccgatgcaattgacggagctaatcaattctatatggggcggtttctccggtccagtggacccaactttcatcatattttctactttagtggaaacataacccgtgaagcgacgccgtttcttttatcatgtccatgtgataaattatgtttttgttatatggtagggttagctgagagctatcaaaagactcttttttatccacctaatagatttgatttgtaacgttaagagcataggaagtcaatttaatcgttactaattacatgcataagaactagtataactatataaggagctccttctagaaattaaatgaaggatgatagaatctagataatcagaaattttactattgatcaatctagctatctcgtagttcaaaaagctttatcgttaacaagtaacaactttcaagtattgcccaaatagataaggttcataacttcatattttttatttattttatgtgtaaaagagtgacagtctatattattctagggggaggacaaggctcatgacataggacaagagaaagaaaaatatagaagcatatagtatattagggtcggtccaaatgaaaacaacgtttaggtatggggcggcgaggctaagttaaattaaccacaaaactccattatcaaccataattttagaattaaaaggtctctgttcctattgatagctccacaatcattcttttaaataatcagaatctcaaataagttcatctttagttacagatttgtatcaatagttgaagttgaaaccaaaataataataatttagttatagttaattttgtcaacaaaacaataccttaactatcatattatgacaaacactaattgagatgaaaaactcttagcagtagctaattcttactatcatcagttaattatactaatgtatatggaaattctgcttaaacaaaaaaaaaacagtggaacatgaatatattaagcaaaatcagtttctattgattatgtagcaatgattagattggtttagattatatatcatcatgacagctagctaggtaattaattagtgaaagaaagtttccacaaaaataatcataatcgtcatacacacaattctatattcatttcattgaaacgaataataaaaacaaccataagcctaccaaaaggaaaacattatcgtaatataatcaatcaataacacgtatacaattattaacgtatattgacaagcaaaattaatgagagcactcactatagctatagtctctctatataaacaactttcattcgtctcttcagtcttcacaaacacaacatatccacaatacaaaacacaactttcatatataacaaaaaaagttatagaaatggccaaagacgtggaaggacctgagggatttcagacaagagactacgaagatccgccaccaactccgtttttcgat

SEQ ID NO: 42 - DNA, 1024 bp, Arabidopsis thaliana
tgcttaccacagattgtgttcctttgtagtaatcgggcttgtagcgcccattttcatactgcccaccactctccatcctcttactttcaactgcaatggagaaattgatatcaaacattgtgaaactaggctgacgagtaactaaaaacagaaatactccaaatcataagcagttccataacatacatttaacccaaataaatcgagaaatcgtatcatatcccacaagtcagcgtaataccatccaaaccaaacgatgaagaaaacaatggagcaagtaagatacgcgggaacatatatagagttcgaatttcaagttaaagcaacgacgagagagctcccagaagaaccaaaattcgaagaaaatgaaaattgtagagagaaaaacttggcatgctgaaattaacagataggtcaagaacacgattaacgatcgaagacttacggatttcaacgagccttcaggagaaacaagcaacggaaatcgagaaagatctgaggatacttggaaatggtgtctgtgtaatgtggcaagaagtggaagacgagccaggtactctcggttcaatttactaatatacccttgtcttaaaactgctaaacgagagcaagcaagaaggttattattgtctatccatcttactcgtaaaaatgcaaagacgtttctgtttcaatctctccaaatataagccaaacaggatatgattttggttctggtggatcattctagtgggccgtatgatgggcctaagaataaggcaactaatctgggtcgaatacgggtagacccgggttgagatcccgacgtgtgcgcttcgctgttgtagtagtagtatatctcatcatcaatcaggcttttgagcctcggaaactcaatccttgtatattcaacggagagagatctgcgagagaaagagagatcagattccggtgttccaaggaagcacatattttaagatccagaatcgcctgagagatgtctaccttcagtggcgatgagaccgctcctttcttcggcttcctt

SEQ ID NO: 43 - protein, 14 aa, Physcomitrella patens
GSGDKRHENLDEDG

SEQ ID NO: 44 - protein, 14 aa, Physcomitrella patens
GKPLSKWERNDAEK

SEQ ID NO: 45 - DNA, 21 bp, Artificial Sequence
  Synthetic primer
aaggcattacctgggagtgga

SEQ ID NO: 46 - DNA, 20 bp, Artificial Sequence
  Synthetic primer
acagcatgggtgcggattct

SEQ ID NO: 47 - DNA, 25 bp, Artificial Sequence
  Synthetic primer
tggttgatctcgttgtgcaggtctc

SEQ ID NO: 48 - DNA, 24 bp, Artificial Sequence
  Synthetic primer
gtcagccaagtcaacaactctctg

SEQ ID NO: 49 - DNA, 20 bp, Artificial Sequence
  Synthetic primer
atgtgggtcagggtatggaa

SEQ ID NO: 50 - DNA, 24 bp, Artificial Sequence
  Synthetic primer
ccgacaaccttcttagtctcctct

SEQ ID NO: 51 - DNA, 23 bp, Artificial Sequence
  Synthetic primer
gagttcttcacgcgatacctcca

SEQ ID NO: 52 - DNA, 27 bp, Artificial Sequence
  Synthetic primer
gaccacctttattaaccccatttacca

SEQ ID NO: 53 - DNA, 23 bp, Artificial Sequence
  Synthetic primer
tggcgaacgctggtcctaataca

SEQ ID NO: 54 - DNA, 24 bp, Artificial Sequence
  Synthetic primer
caaaaactcctctgccccaatcaa

SEQ ID NO: 55 - DNA, 20 bp, Artificial Sequence
  Synthetic primer
gagttcacggaagcggagag

SEQ ID NO: 56 - DNA, 21 bp, Artificial Sequence
  Synthetic primer
atatctttcaggctccaccga

SEQ ID NO: 57 - DNA, 23 bp, Artificial Sequence
  Synthetic primer
gccaagaagaaggtaatagtgcg

SEQ ID NO: 58 - DNA, 19 bp, Artificial Sequence
  Synthetic primer
acgtctgcctcgctctagc

SEQ ID NO: 59 - DNA, 21 bp, Artificial Sequence
  Synthetic primer
gcgaagagcgagtatgacgag

SEQ ID NO: 60 - DNA, 23 bp, Artificial Sequence
  Synthetic primer
agccacgaatctaacttgtgatg

SEQ ID NO: 61 - DNA, 21 bp, Artificial Sequence
  Synthetic primer
cgtccaggaacagtcgctctt

SEQ ID NO: 62 - DNA, 21 bp, Artificial Sequence
  Synthetic primer
ttcacagcctacgccctctct

SEQ ID NO: 63 - DNA, 22 bp, Artificial Sequence
  Synthetic primer
caacgacgatacttcttggctg

SEQ ID NO: 64 - DNA, 21 bp, Artificial Sequence
  Synthetic primer
gctgctccaccagtcctgcta

SEQ ID NO: 65 - DNA, 20 bp, Artificial Sequence
  Synthetic primer
gggctgctagcttcaacatc

SEQ ID NO: 66 - DNA, 20 bp, Artificial Sequence
  Synthetic primer
ttgattgcagccttgatctg

SEQ ID NO: 67 - DNA, 21 bp, Artificial Sequence
  Synthetic primer
cgaccagggcaaccgcaccac

SEQ ID NO: 68 - DNA, 21 bp, Artificial Sequence
  Synthetic primer
acggtgttgatggggttcatg

SEQ ID NO: 69 - DNA, 21 bp, Artificial Sequence
  Synthetic primer
gtgaggctggtgctgattacg

SEQ ID NO: 70 - DNA, 22 bp, Artificial Sequence
  Synthetic primer
tggtgcagctagcatttgagac

SEQ ID NO: 71 - DNA, 22 bp, Artificial Sequence
  Synthetic primer
cctgtcgtgtcgtcggtctaaa

SEQ ID NO: 72 - DNA, 22 bp, Artificial Sequence
  Synthetic primer
acgcagatccagcagcctaaag

SEQ ID NO: 73 - DNA, 20 bp, Artificial Sequence
  Synthetic primer
agtgtcctgtccacccactc

SEQ ID NO: 74 - DNA, 20 bp, Artificial Sequence
  Synthetic primer
agcatgaagtggatccttgg

SEQ ID NO: 75 - DNA, 22 bp, Artificial Sequence
  Synthetic primer
gaccactgtcggcagaggcatc

SEQ ID NO: 76 - DNA, 22 bp, Artificial Sequence
  Synthetic primer
gaccactgtcggcagaggcatc

SEQ ID NO: 77 - DNA, 21 bp, Artificial Sequence
  Synthetic primer
ggcaatggaatccgaggaggt

SEQ ID NO: 78 - DNA, 23 bp, Artificial Sequence
  Synthetic primer
ggtatcagagccatgaataggtc

SEQ ID NO: 79 - DNA, 19 bp, Artificial Sequence
  Synthetic primer
cgacgacgatcctgtgctc

SEQ ID NO: 80 - DNA, 22 bp, Artificial Sequence
  Synthetic primer
ccgttgacagttgagtaacacc

SEQ ID NO: 81 - DNA, 30 bp, Artificial Sequence
  Synthetic primer
aaaggatccggggacttgttgacgttcgag

SEQ ID NO: 82 - DNA, 29 bp, Artificial Sequence
  Synthetic primer
aaaaagcttttgatggccttggaaatctt

(19) United States
(12) Patent Application Publication
(10) Pub. No.: US 2008/0020464 A1
(43) Pub. Date: Jan. 24, 2008

(54) Vascular plants expressing Na+ pumping ATPases

(75) Inventors: Andrew Jacobs, Stirling (AU); Christina Lunde, Copenhagen (DK); Mark Tester, Toorale Gidas (AU)

Correspondence Address: BIRCH STEWART KOLASCH & BIRCH, PO BOX 747, FALLS CHURCH, VA, 22040-0747, US

(73) Assignee: Australian Centre for Plant Functional Genomics Pty Ltd, Glen Osmond (AU)

(21) Appl. No.: 11/783,064

(22) Filed: Apr. 5, 2007

Related U.S. Application Data
(63) Continuation-in-part of application No. PCT/AU05/01553, filed on Oct. 7, 2005.
(60) Provisional application No. 60/616,218, filed on Oct. 7, 2004.

(51) Int. Cl. A01H 11/00; C12N 15/82; C12N 5/00

(52) U.S. Cl. 435/410; 435/420; 800/278; 800/295

(57) ABSTRACT

The present invention relates to a vascular plant including cells expressing a Na⁺ pumping ATPase.

This application is a Continuation-In-Part of co-pending Application No. PCT/AU2005/001553 filed on Oct. 7, 2005, This application also claims priority under 35 U.S.C. § 119(a) on Provisional Patent Application No(s). 60/616,218 filed on Oct. 7, 2004. The entire contents of each of the above documents is hereby incorporated by reference.

### FIELD OF THE INVENTION

The present invention relates to vascular plants, and cells from vascular plants, expressing a Na⁺ pumping ATPase.

The present invention also relates to methods of improving Na⁺ secretion and Na⁺ tolerance in vascular plants, and in cells from vascular plants, by the expression of a Na⁺ pumping ATPase in the plants or cells.

### BACKGROUND OF THE INVENTION

High concentrations of salts in soils account for large decreases in the yield of a wide variety of crops all over the world. Almost 1,000 million ha of land is affected by soil salinity, which represents 7% of all land area. Of the 1.5 billion hectares that is currently cultivated, about 5% (77 million ha) is salt affected. The problem of soil salinity is only likely to worsen, given that current farming methods are contributing to the salinisation of water sources.

Saline solutions impose both ionic and osmotic stresses on plants. These stresses can be distinguished at several levels. In particular, Na⁺-specific damage is associated with the accumulation of Na⁺ in leaf tissues and results in necrosis of older leaves, starting at the tips and margins and working back through the leaf. Growth and yield reduction occur due to the shortening of the lifetime of individual leaves, thus reducing net productivity and crop yield.

In the shoots, high concentrations of Na⁺ can cause a range of problems for the plant, both osmotic and metabolic. Leaves are more vulnerable to Na⁺ than roots, simply because Na⁺ accumulates to higher concentrations in the shoots than in the roots. Roots tend to maintain fairly constant levels of Na⁺ over time, and can regulate Na⁺ levels by export to the soil or to the shoot. Na⁺ is transported to the shoots in the rapidly moving transpiration stream in the xylem, but can only be returned to the roots in the phloem. There is limited evidence of recirculation of shoot Na⁺ to the roots, suggesting that Na⁺ transport is largely unidirectional and results in progressive accumulation of Na⁺ as the leaves age.

A number of different mechanisms may be used to improve tolerance to salinity. Intracellular compartmentation of Na⁺ in cells, intraplant allocation of Na⁺ and exclusion of Na⁺ from the whole plant may each improve tolerance to salinity. Such processes represent adaptation to Na⁺ at two levels of organisation: those that confer tolerance to cells per se, and those that contribute to the tolerance of plants as a whole.

Compartmentalisation of Na⁺ in vacuoles is one example of how the tolerance of cells to Na⁺ may be improved. However, although cells with improved Na⁺ tolerance may be selected in vitro, there has been a persistent inability to generate vigorous Na⁺ tolerant plants from such tolerant cells.

As can be appreciated from the preceding discussion, there is a need to identify new methods of improving the tolerance of plants to Na⁺, and to produce plants with improved tolerance to Na⁺. The present invention relates to vascular plants, and cells from vascular plants, which express a Na⁺ pumping ATPase. The present invention also relates to methods of improving Na⁺ secretion and Na⁺ tolerance in vascular plants, and in cells from vascular plants, by expressing a Na⁺ pumping ATPase in the plants or cells.

A reference herein to a patent document or other matter which is given as prior art is not to be taken as an admission that that document or matter was, in any country, known or that the information it contains was part of the common general knowledge as at the priority date of any of the claims.

### SUMMARY OF THE INVENTION

The present invention provides a vascular plant including cells expressing a Na⁺ pumping ATPase.

The present invention also provides a cell from a vascular plant, the cell expressing a Na⁺ pumping ATPase.

The present invention further provides a method of increasing Na⁺ secretion from a cell from a vascular plant, the method including the step of expressing a Na⁺ pumping ATPase in the cell.

The present invention also provides a cell from a vascular plant, the cell having increased Na⁺ secretion due to expression of a Na⁺ pumping ATPase in the cell.

The present invention also provides a vascular plant including cells with increased Na⁺ secretion, the increased Na⁺ secretion of the cells due to expression of a Na⁺ pumping ATPase in the cells.

The present invention also provides a method of improving the Na⁺ tolerance of a cell from a vascular plant, the method including the step of expressing a Na⁺ pumping ATPase in the cell.

The present invention also provides a cell from a vascular plant, the cell having improved tolerance to Na⁺ due to expression of a Na⁺ pumping ATPase in the cell.

The present invention also provides a method of improving the Na⁺ tolerance of a vascular plant, the method including the step of expressing a Na⁺ pumping ATPase in cells of the plant.

The present invention also provides a vascular plant with improved tolerance to Na⁺, the improved tolerance to Na⁺ due to expression of a Na⁺ pumping ATPase in cells of the plant.

The present invention arises from the identification that the management of Na⁺ movement within a plant by the expression of exogenous Na⁺ transporters is likely to be a more effective means of improving the Na⁺ tolerance of plants than the manipulation of endogenous Na⁺ transporters. The present invention is based upon the isolation of nucleic acids that encode Na⁺ pumping ATPases from non-animal eukaryotes such as the moss, Physcomitrella patens, and the yeast Saccharomyces cerevisiae, and the introduction of such nucleic acids into vascular plants, which do not appear to encode Na⁺ pumping ATPases. The expectation is that the expression of a Na⁺ pumping ATPase in such plants will result in improved secretion of Na⁺ from their cells and that the plants will show improved tolerance to Na⁺.

Various terms that will be used throughout the specification have meanings that will be well understood by a skilled addressee. However, for ease of reference, some of these terms will now be defined.

The term “plant” as used throughout the specification is to be understood to include whole plants, parts of plants, plant organs (e.g., leaves, stems, roots, etc.), seeds and the progeny of any of the aforementioned.

The term “vascular plant” as used throughout the specification is to be understood to mean any plant that has a specialized conducting system, generally consisting of phloem (food-conducting tissue) and xylem (water-conducting tissue).

The term “tolerance”, or variants thereof as used throughout the specification in relation to plants and plant cells, is to be understood to mean the ability of a plant or plant cell to display an improved response to an increase in extracellular and/or intracellular Na⁺ concentration, as compared to a similar plant or cell not expressing a Na⁺ pumping ATPase. A plant with improved tolerance to Na⁺ may for example show an improved growth rate, or a decreased level of necrosis in the leaves, when subjected to an increase in Na⁺ concentration, as compared to a similar plant.

The term “nucleic acid” as used throughout the specification is to be understood to mean to any oligonucleotide or polynucleotide. The nucleic acid may be DNA or RNA and may be single stranded or double stranded. The nucleic acid may be any type of nucleic acid, including for example a nucleic acid of genomic origin, cDNA origin (i.e. derived from a mRNA) or of synthetic origin. In this regard, the term “polynucleotide” refers in general to polyribonucleotides and polydeoxyribonucleotides, and can denote an unmodified RNA or DNA, a modified RNA or DNA, or any other modifications to the bases, sugar or phosphate backbone that are functionally equivalent to the nucleotide sequence.

The term “amino acid sequence” refers to an oligopeptide, peptide, polypeptide, or protein sequence, and fragments or portions thereof, and to naturally occurring, recombinant, mutated or synthetic polypeptides.

The term “amplification” or variants thereof as used throughout the specification is to be understood to mean the production of additional copies of a nucleic acid sequence. For example, amplification may be achieved using polymerase chain reaction (PCR) technologies, essentially as described in Dieffenbach, C. W. and G. S. Dveksler (1995) PCR Primer, a Laboratory Manual, Cold Spring Harbor Press, Plainview, N.Y.

The term “hybridization” or variants thereof as used throughout the specification is to be understood to mean any process by which a strand of nucleic acid binds with a complementary strand through base pairing. Hybridization may occur in solution or between one nucleic acid sequence present in solution and another nucleic acid sequence immobilized on a solid support (e.g., membranes, filters, chips etc). In this regard, stringent conditions for detecting complementary nucleic acids are conditions that allow complementary nucleic acids to bind to each other within a range from at or near the Tm (Tm is the melting temperature) to about 20° C. below Tm. Factors such as the length of the complementary regions, type and composition of the nucleic acids (DNA, RNA, base composition), and the concentration of the salts and other components (e.g. the presence or absence of formamide, dextran sulfate and/or polyethylene glycol) must all be considered, essentially as described in Current Protocols in Molecular Biology, John Wiley & Sons, N.Y. (1989).

### BRIEF DESCRIPTION OF THE FIGURES

The patent or application file contains at least one drawing executed in color. Copies of this patent or patent application publication with color drawing(s) will be provided by the office upon request and payment of the necessary fee.

FIG. 1 shows the plasmid map of pTOOL2.

FIG. 2 shows the plasmid map of pAJ21.

FIG. 3 shows the plasmid map of pGreenII0229UAS⁺Nos5A.

FIG. 4 shows the plasmid map of pDP1.

FIG. 5 shows the plasmid map of pPG1.

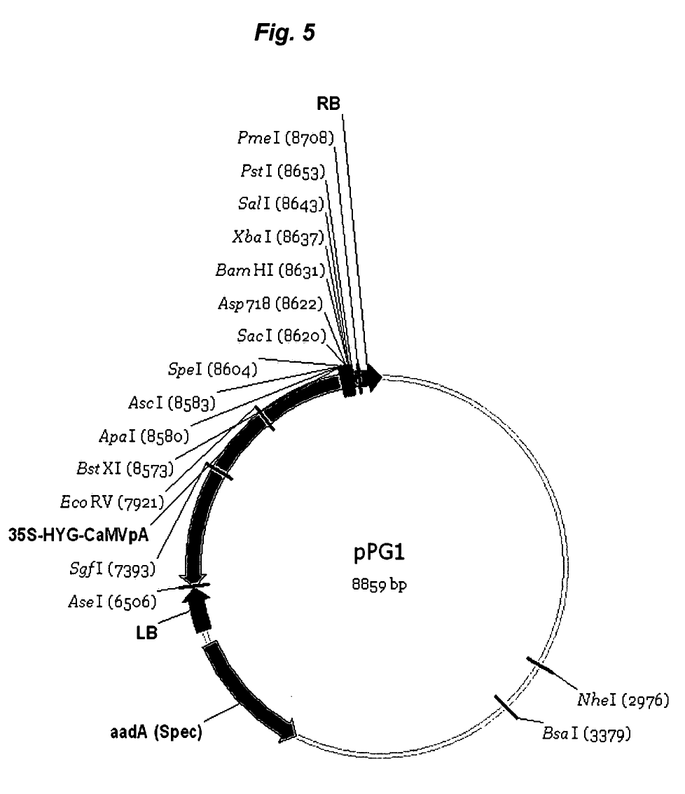

FIG. 6 shows the plasmid map of pJIT145-Kan.

FIG. 7 shows the plasmid map of T-Easy 35S-Hyg.

FIG. 8 shows the plasmid map of pAJ40.

FIG. 9 shows the plasmid map of pAJ41.

FIG. 10 shows Gal induced transcription of PpENA1 rescues the B31 mutant's salt sensitivity phenotype. B31 Salt-sensitive yeast were transformed with PpENA1 under control of the Gal promoter inoculated onto 300 mM NaCl plates. B31 (MAT a ade2 ura3 leu2 his3 trp1 ena1Δ::HIS3::ena4Δ nha1Δ::LEU2 transformed with pYES-ENA) was grown in SC-ura overnight. 5 serial 1 in 2 dilutions were made and 1 μl of each spotted on to either SC-ura+0.3M NaCl+glucose or SC-ura+0.3M NaCl+galactose.

FIG. 11 shows the results of confirmation by PCR of PpENA1 disruption in genomic DNA from kanamycin resistant Physcomitrella patens transformants. PCR check of the 5′ end was done using oCL148-oCL76 for the first PCR and oCL149-oCL100 for the second PCR. The PCR check of the 3′ end was done using PpENA1R-oCL77 for the first PCR and oCL101-PpENA1R for the second. The expected size of the fragment if insertion occurred was 1429 bp and 1835 bp.

FIG. 12 shows PpENA1 mRNA levels was determined by qPCR for three moss PpENA1 knockout mutants and wildtype.

FIG. 13 shows sodium and potassium concentrations in wildtype and mutant moss as determined by flame photometry. Bars represent standard deviation.

FIG. 14 shows that PpENA1 knockout mutants have reduced biomass in comparison to wildtype after 1 week on media containing 100 or 200 mM NaCl

FIG. 15 shows a graphic representation of the colony diameter for wildtype and PpENA1 knockout mutants after 1 week on containing 100 or 200 mM NaCl.

FIG. 16 shows PpENA1 mRNA levels in Arabidopsis T1 transgenics constitutively expressing PpENA1.

FIG. 17 shows PpENA1 mRNA levels in Arabidopsis T1 transgenics induced following exposure to 30 mM NaCl.

FIG. 18 shows PpENA1 mRNA levels in Arabidopsis T1 transgenics induced following exposure to 30 mM NaCl. The Arabidopsis VHAc3 promoter produces a low level of expression of PpENA1 mRNA at 30 mM NaCl.

FIG. 19 shows that T3 Transgenic Arabidopsis plants constitutively expressing PpENA1 may have a growth advantage on 100 mM NaCl when compared to wildtype.

FIG. 20 shows the expression of PpENA1 in rice.

FIG. 21 shows hygromycin resistant barley plants transformed with pAJ54 and pAJ55 in tissue culture.

FIG. 22 shows Western analysis of Arabidopsis T2 transgenics and salt treated moss probed with PpENA1 antibody.

FIG. 23 shows the correlation of PpENA1 expression with shoot Na⁺ concentration in T1 rice.

FIG. 24 shows the correlation of PpENA1 expression with root Na⁺ concentration in T1 rice.

FIG. 25 shows the level of expression of PpENA1 in shoots of various T1 barley lines.

FIG. 26 shows shoot and root Na⁺ concentrations measured by flame photometry. 1-B6 and 1-C1 are lines expressing PpENA1 in the root epidermis. 4-B1 and 4-C1 are lines expressing PpENA1 in the root xylem parenchyma. 10-yeb-YEB harvested after 10 mM Na⁺ stress; 50-yeb-YEB harvested after 50 mM Na⁺ stress; 50-old-leaf blade harvested after 50 mM Na⁺ stress.

### GENERAL DESCRIPTION OF THE INVENTION

As described above, in one embodiment the present invention provides a vascular plant including cells expressing a Na⁺ pumping ATPase.

This embodiment of the present invention is directed to a vascular plant in which some or all of the cells in the plant express a Na⁺ pumping ATPase. Na⁺ pumping ATPases are a class of membrane bound proteins that actively pump Na⁺ ions out of cells, and which do not appear to exist in vascular or flowering plants. They belong to the P-type superfamily of ATP-driven pumps, and in particular to a separate phylogenetic group, the type IID ATPases.

The vascular plant according to this embodiment of the present invention may be a plant in which all the cells in the plant express a Na⁺ pumping ATPase. Alternatively, the vascular plant according to the present invention may also be a plant in which only a subset of the cells that constitute the plant express a Na⁺ pumping ATPase.

Examples of vascular plants suitable for the present invention include alfalfa, almond, apple, apricot, arabidopsis, artichoke, atriplex, avocado, barley, beet, birch, brassica, cabbage, cacao, canola, cantaloup/cantalope, carnations, cassawa, castorbean, caulifower, celery, clover, coffee, corn, cofton, cucumber, garlic, grape, grapefruit, hemp, hops, lettuce, lupins, maple, medics, melon, mustard, oak, oat, olive, onion, orange, pea, peach, pear, pepper, pine, plum, poplar, potato, prune, radish, rape, rice, rose, rye, sorghum, soybean, spinach, squash, strawberries, sunflower, sweet corn, tobacco, tomato and wheat.

The vascular plant may be a dicot plant or a monocot plant. In one embodiment, the vascular plant is a monocot plant. In one specific embodiment, the monocot plant is a cereal crop plant such as wheat, barley, rye, corn, rice and pasture grasses such as ryegrass.

The Na⁺ pumping ATPase is expressed in cells of the vascular plant from a suitable nucleotide sequence operative in expressing a Na⁺ pumping ATPase introduced into the cells.

Accordingly, in another embodiment the present invention provides a cell from a vascular plant, the cell including a nucleotide sequence encoding a Na⁺ pumping ATPase. In another embodiment, the present invention provides a vascular plant including cells that include a nucleotide sequence encoding a Na⁺ pumping ATPase.

Methods for transforming nucleic acids into plant cells are known in the art. For example, Agrobacterium tumefaciens-mediated transformation or particle-bombardment-mediated transformation may be used to transform plants, depending upon the plant species. A suitable method for transformation of plants by Agrobacterium is described in Clough, S. J. and Bent, A. F. (1998) Plant Journal 16:735-743. A suitable method for transformation using particle bombardment is as described in Klein et al. (1988) Proc. Natl. Acad. Sci. 85(12):4305-4309.

The Na⁺ pumping ATPase of the present invention may be derived from a suitable organism containing a nucleotide sequence encoding a Na⁺ pumping ATPase, such as a moss or a yeast.

Other organisms having a gene encoding a Na⁺ pumping ATPase include Lieshmania donovani, Neurospora crassa, Schizosaccharomyces pombe, Zygosaccharomyces rouxi, and Saccharomyces occidentalis, Fusarium oxysporum, Dunaliella maritima and Tetraselmis viridis.

In the case of a moss Na⁺ pumping ATPase, in one embodiment the Na⁺ pumping ATPase is from the genus Physcomitrella. In one specific embodiment, the Na⁺ pumping ATPase is from Physcomitrella patens.

In this regard, Physcomitrella patens appears to encode two Na⁺ pumping ATPases, ENA1 and ENA2. The nucleotide sequence corresponding to the ENA1 gene of Physcomitrella patens mRNA is described in GenBank Accession No. AJ564254, and is designated SEQ ID NO.1. The amino acid sequence of the ENA1 ATPase is designated SEQ ID NO.2.

The ENA2 gene appears to produce three alternative mRNAs (ENA2A, ENA2B and ENA2C) due to alternative splicing at the 3′ end. The nucleotide sequence encoding the ENA2 gene is described in GenBank Accession No. AJ564259, and is designated SEQ ID NO. 3.

The nucleotide sequence corresponding to the mRNA of the ENA2 splice variant 2A of Physcomitrella patens is described in GenBank Accession No. AJ564259, and is designated SEQ ID NO. 4. The amino acid sequence of the protein encoded by the ENA2A splice variant is designated SEQ ID NO. 5.

The nucleotide sequence corresponding to the ENA2B gene is described GenBank Accession No. AJ564260, and is designated SEQ ID NO.6. The nucleotide sequence corresponding to the mRNA of the ENA2 splice variant 2B of Physcomitrella patens is described in GenBank Accession No. AJ564260, and is designated SEQ ID NO.7: The amino acid sequence of the protein encoded by the ENA2B splice variant is designated SEQ ID NO. 8.

The nucleotide sequence corresponding to the ENA2C gene is described in GenBank Accession No. AJ564261, and is designated SEQ ID NO.9: The nucleotide sequence corresponding to the mRNA of the ENA2 splice variant 2C is described in GenBank Accession No. AJ564261, and is designated SEQ ID NO. 10. The amino acid sequence of the protein encoded by the ENA2C splice variant is designated SEQ ID NO.11.

In the case of a yeast Na⁺ pumping ATPase, in one embodiment the Na⁺ pumping ATPase is from the genus Saccharomyces. In one specific embodiment, the Na⁺ pumping ATPase is from Saccharomyces cerevisiae.

In this regard, Saccharomyces cerevisiae appears to encode three Na⁺ pumping ATPases, ENA1, ENA2 and ENA5. The nucleotide sequence corresponding to the ENA1 gene of Saccharomyces cerevisiae is described in GenBank Accession Z74336. The nucleotide sequence corresponding to the mRNA is described in GenBank Accession Z74336, and is designated SEQ ID NO.12. The amino acid sequence of the ENA1 ATPase is designated SEQ ID NO. 13.

The nucleotide sequence corresponding to the ENA2 gene of Saccharomyces cerevisiae is described in GenBank Accession Z74335. The nucleotide sequence corresponding to the mRNA is also described in GenBank Accession Z74335, and is designated SEQ ID NO.14. The amino acid sequence of the ENA2 ATPase is designated SEQ ID NO.15.

The nucleotide sequence corresponding to the ENA5 gene of Saccharomyces cerevisiae is described in GenBank Accession Z74334. The nucleotide sequence corresponding to the mRNA is also described in GenBank Accession Z74334, and is designated SEQ ID NO.16. The amino acid sequence of the ENA2 ATPase is designated SEQ ID NO.17.

In the case of Na⁺ pumping ATPases from other species, a nucleotide sequence encoding a Na⁺ pumping ATPase may be identified by a method known in the art. For example, reverse transcription-PCR (RT-PCR) using primers that will amplify nucleotide sequences containing a Na⁺ pumping ATPase may be used to isolated genes that encode type II P-type ATPases. A suitable method is described in Benito et al. (2000) Mol. Micro. 35(5): 1079-1088.

Alternatively, colony hybridization of a genomic library using nucleotide sequences that detect a Na⁺ pumping ATPase may be used to isolate cDNAs or genes that encode type II P-type ATPases. A suitable method for performing colony hybridization is described in Sambrook, J, Fritsch, E. F. and Maniatis, T. Molecular Cloning: A Laboratory Manual 2nd. ed. Cold Spring Harbor Laboratory Press, New York. (1989).

In this case of using a hybridization technique to identify a nucleotide sequence encoding a Na⁺ pumping ATPase, it should be noted that absolute complementarity, although preferred, is not required, as long as the detectably labelled probe is able to hybridize under stringent conditions to the target nucleic acid. As will be appreciated, the ability to hybridize will depend on both the degree of complementarity and the length of the probe. Methods known in the art may be used to formulate possible probes, and to prepare and label the probes. Methods generally for the preparation and labelling of probes are described in Sambrook, J, Fritsch, E. F. and Maniatis, T. Molecular Cloning: A Laboratory Manual 2nd. ed. Cold Spring Harbor Laboratory Press, New York. (1989).

An alternative method to identifying a nucleotide sequence encoding a Na⁺ pumping ATPase is by identifying a nucleotide sequence that shows significant sequence similarity or homology to known Na⁺ pumping ATPases.

In this regard, various algorithms exist for determining the degree of homology between any two proteins or any two nucleotide sequences. For example, the BLAST algorithm can be used for determining the extent of homology between two nucleotide sequences (blastn) or the extent of homology between two amino acid sequences (blastp). BLAST identifies local alignments between the sequences in the database and predicts the probability of the local alignment occurring by chance. The BLAST algorithm is as described in Altschul et al., 1990, J. Mol. Biol. 215:403-410.

In one embodiment, a nucleotide sequence encoding a Na⁺ pumping ATPase has greater than 90% similarity with Physcomitrella patents ENA1. In one specific embodiment, a nucleotide sequence encoding a Na⁺ pumping ATPase has greater than 95% similarity with Physcomitrella patents ENA1.

The cDNAs or genes may then be isolated and cloned by methods known in the art, such as described in Sambrook, J, Fritsch, E. F. and Maniatis, T. Molecular Cloning: A Laboratory Manual 2nd. ed. Cold Spring Harbor Laboratory Press, New York. (1989).

Upon isolation and cloning of the candidate cDNAs or genes, the ability of the nucleotide sequences to express a functional Na⁺ pumping ATPase may be confirmed by a suitable method known in the art.

For example, the ability of a nucleotide sequence to express a functional Na⁺ pumping ATPase may be confirmed by the ability of the protein encoded by the nucleotide sequence to reduce Na⁺ concentration in a suitable cell.

Determination of the intracellular concentration of Na⁺ may be performed by a method known in the art, such as by determination of intracellular concentrations of Na⁺ may be by flame photometry, as described in Essah et al. (2003) Plant Physiology 133: 307-318.

Alternatively, the ability of a nucleotide sequence to suppress the Na⁺ sensitive defect in a Saccharomyces cerevisiae Na⁺ pumping ATPase null mutant may be determined, as described for example in Benito et al., (2000) Mol. Microbiol. 35: 1079-1088. In a similar fashion, the ability of nucleotide sequence to suppress the Na⁺ sensitivity of Physcomitrella patents containing inactive ENA1 and/or ENA2 genes may be determined.

Alternatively, the Na⁺ pumping ATPase in the various embodiments of the present invention may be derived from a nucleotide sequence encoding a non-naturally occurring Na⁺ pumping ATPase, such as a nucleotide sequence encoding a synthetic Na⁺ pumping ATPase, a chimeric Na⁺ pumping ATPase, or a Na⁺ pumping ATPase that is a variant of a naturally occurring Na⁺ pumping ATPase.

Examples of variants include modifications of the nucleotide sequence to alter codon usage, variants that encode a biologically active fragment of a naturally occurring Na⁺ pumping ATPase, or variants that delete, substitute or add one or more amino acids to the coding region of a naturally occurring Na⁺ pumping ATPase.

Altering the codon usage of the nucleotide sequence encoding the Na⁺ pumping ATPase may be used to improve expression of a gene in a vascular plant. For example, for the expression of a Na⁺ pumping ATPase in a cereal plant, the codon usage in the coding sequence of the progenitor gene or cDNA may be altered to more closely reflect the codon preference in cereals. By way of example, comparison of the codon usage between Physcomitrella patens and various cereals is shown in Example 8, Table 2. In this case, the codon usage for the amino acids cysteine, phenylalanine, glycine, serine and threonine may be modified to more closely reflect the codon usage in cereals generally, or even the particular cereal of interest.

By way of another example, for the expression of a Na⁺ pumping ATPase in a dicot plant, the codon usage in the coding sequence of the progenitor gene or cDNA may be altered to more closely reflect the codon preference in dicots.

Comparison of the codon usage between Physcomitrella patens and various dicots is shown in Example 8, Table 3. In this case, the codon usage for the amino acids aspartic acid, arginine and valine may be modified to more closely reflect the codon usage in dicots.

A biologically active fragment of a naturally occurring Na⁺ pumping ATPase is a polypeptide having similar structural, regulatory, or biochemical functions as that of the full size protein. Biologically active fragments may be amino or carboxy terminal deletions of a protein or polypeptide, an internal deletion of a protein or polypeptide, or any combination of such deletions. A biologically active fragment will also include any such deletions fused to one or more additional amino acids.

For example, a biologically active fragment of the PpENA1 gene is a deletion of 255 amino acids at the amino-terminus of the protein, or a truncation of the last 187 amino acids from the carboxy terminus of the protein.

Accordingly, in another embodiment the present invention provides a vascular plant including cells expressing a Physcomitrella patens Na⁺ pumping ATPase, wherein the Na⁺ pumping ATPase is a 255 amino acid amino terminus deletion or a 187 amino acid carboxy terminus deletion of the ENA1 protein. In yet another embodiment, the present invention provides a cell from a vascular plant, the cell expressing a Physcomitrella patens Na⁺ pumping ATPase, wherein the Na⁺ pumping ATPase is a 255 amino acid amino terminus deletion or a 187 amino acid carboxy terminus deletion of the ENA1 protein.

In the case of a variant that is a modification to substitute one or more amino acids, the variant may have “conservative” changes, wherein a substituted amino acid has similar structural or chemical properties to the replaced amino acid (e.g., replacement of leucine with isoleucine). Alternatively, a variant may also have “non-conservative” changes (e.g., replacement of a glycine with a tryptophan), or a deletion and/or insertion of one or more amino acids.

In the case where the variant is a chimeric Na⁺ pumping ATPase, the nucleotide sequence encoding the chimera may be derived from different Na⁺ pumping ATPases in a particular species, or may be derived from Na⁺ pumping ATPases from different species.

The variant may also be a splice variant of a naturally occurring gene. For example, the splice variant may be a naturally occurring splice variant or a variant engineered to express an alternatively spliced form of the mRNA encoding a Na⁺ pumping ATPase.

As will be appreciated, in order to achieve expression of the Na⁺ pumping ATPase in the cells of the plant, the nucleotide sequence encoding the Na⁺ pumping ATPase will be operably linked to a suitable promoter. In one embodiment, the level of expression of the Na⁺ pumping ATPase in the cells results in increased secretion of Na⁺ from the cells and/or increased tolerance to Na⁺, as compared to cells not expressing a Na⁺ pumping ATPase.

For example, the promoter may be a promoter endogenous to the plant of interest, a promoter from another plant, the promoter normally associated with the nucleotide sequence encoding the Na⁺ pumping ATPase in the organism from which the Na⁺ pumping ATPase is isolated (so long as the promoter is sufficiently active in the vascular plant of interest), a promoter from another organism that is active in the vascular plant of interest (such as a Ti plasmid promoter), a viral promoter active in the vascular plant of interest, a chimeric promoter, or a synthetic promoter.

The promoter may further be a constitutive promoter, an inducible promoter or a cell specific promoter.

Examples of constitutive promoters include the 35S promoter of cauliflower mosaic virus, the nopaline synthase promoter, the ubiquitin promoter, the actin promoter and viral PS4 promoter.

Examples of inducible promoters include Na⁺ inducible promoters (i.e. up-regulated in response to salt stress) such as the PIP2.2, PpENA1 and VHA-c3 promoters, drought-inducible promoters such as Bnuc, Dhn8, Rd17, heat shock-inducible promoters such as hsp1, metal ion-inducible promoters such as metallothionen, or promoters active in plants that are repressed by bacterial or plasmid operator/repressors systems, such as the Gal4, lacOllacl or tetO/tetR systems, or other inducible promoters such as the alcR promoter, the dexamethasone (dex) promoter, and the NHA1 and NHA(D) promoters.

In the case of an inducible promoter, in one embodiment the promoter is the PIP2.2 promoter or VHA-c3 promoter, a variant of either of these promoters, or another promoter including the DNA elements responsible for the inducibility of these promoters.

Examples of cell-specific promoters depend upon the particular cell type in which expression of the Na⁺ pumping ATPase is desired. For example, the Na⁺ pumping ATPase may be expressed in mature root epidermal cells, to promote exclusion from the root (and thus the plant). However it should also be appreciated that expression of the Na⁺ pumping ATPase in some cells may be detrimental to the plant as a whole. For example, expression of the Na⁺ pumping ATPase stelar cells, where extrusion from cells would increase loading into the xylem vessels and thus increase delivery to the shoot, is likely to be a detrimental process. Other cell types in which it would be desirable to express the Na⁺ pumping ATPase include mature root cortex, leaf and stem trichomes, and hydathodes.

To drive expression in specific cell types that lack a well characterised promoter, enhancer trap lines expressing the yeast transcription factor fusion protein, GAL4:VP16 (as visualised by expression of GFP driven by the GAL4 upstream activation sequence), in specific cell types may be used, as described in Johnson et al. (2005) Plant J. 41(5):779-89.

In one embodiment, the Na⁺ pumping ATPase is expressed in cortical and epidermal root cells of the plant. In this regard, expression of the Na⁺ pumping ATPase in root cells of the plant may be achieved by the use of a suitable constitutive promoter, inducible promoter, or a root cortex-specific promoter.

Various modifications may also be made to the nucleotide sequence encoding the Na⁺ pumping ATPase to regulate its expression. A recombinant nucleic acid molecule for expressing a Na⁺ pumping ATPase may also contain other suitable transcriptional, mRNA stability or translational regulatory elements, known in the art.

For example, the stability of a mRNA encoding the Na⁺ pumping ATPase may also be regulated by modifying the nucleotide sequence encoding the mRNA, such as by introduction into the mRNA of an element that stabilises the mRNA in response to increased Na⁺ concentration.

Translational rates may also be modified. For example, signals providing efficient translation may be introduced into the nucleotide sequence encoding the Na⁺ pumping ATPase.

In addition, under certain circumstances it may be beneficial to introduce one or more intronic sequence into the nucleotide sequence encoding the Na⁺ pumping ATPase, such that the intron is spliced out of the mature mRNA in the vascular plant of interest. Such a construct is particular useful given the difficulty of cloning Na⁺ pumping ATPases in bacteria. The presence of the intronic sequence produces a gene product in bacterial that is non-functional, allowing cloning and manipulation of the nucleotide sequence. The intron is then removed upon expression of the nucleotide sequence in the vascular plant. An example of a suitable intronic sequence is the small Arabidopsis WRKY33 first intron.

The present invention also contemplates isolated nucleic acids as described above.

Accordingly, in one embodiment the present invention provides an isolated nucleic acid including a nucleotide sequence encoding a Na⁺ pumping ATPase, the nucleotide sequence engineered to improve expression of the Na⁺ pumping ATPase in a vascular plant.

The nucleic acids of the present invention may be prepared by a suitable method known in the art. Methods for preparing nucleic acids are as described in Sambrook, J, Fritsch, E. F. and Maniatis, T. Molecular Cloning: A Laboratory Manual 2nd. ed. Cold Spring Harbor Laboratory Press, New York. (1989).

In the case of shorter nucleic acids, such as oligonucleotides, the nucleic acids may also be synthesized by chemical synthesis using a method known in the art. Larger nucleotide sequences may also be prepared by annealing and ligation of a number of oligonucleotides.

In the case of nucleic acids encoding the Na⁺ pumping ATPase, the nucleic may be produced for example by cDNA cloning, genomic cloning, DNA synthesis, polymerase chain reaction (PCR) technology, or a combination of these approaches.

Vectors for introducing nucleic acids into cells are also known in the art. The type of vector selected is dependent upon the specific stage in the overall process of constructing a final nucleic acid for introduction into a plant cell.

Vectors can be constructed by recombinant DNA methods known in the art. Types of vectors include cosmids, plasmids, bacteriophage, baculoviruses and viruses.

The vector may then be introduced into the specific host by a method of transformation known in the art and applicable to the host. Methods for introducing exogenous DNAs into cells are described in Sambrook, J, Fritsch, E. F. and Maniatis, T. Molecular Cloning: A Laboratory Manual 2nd. ed. Cold Spring Harbor Laboratory Press, New York. (1989).

For example, vectors and techniques suitable for the transformation of bacteria or for the transformation of plants are known in the art.

Accordingly, the present invention also provides a cell including any of the above described nucleic acids. Examples of cells include fungal cells, yeast cells, bacterial cells (eg E. coli; Agrobacterium), or plant cells.

Upon construction of a suitable nucleic acid for expressing a Na⁺ pumping ATPase in a vascular plant, the nucleotide sequence encoding the Na⁺ pumping ATPase must then be introduced into a suitable plant cell. For example, Agrobacterium tumefaciens-mediated transformation or particle-bombardment-mediated transformation may be used to transform plant cells, depending upon the plant species.

Accordingly, the present invention also provides a cell from a vascular plant, the cell expressing a Na⁺ pumping ATPase. In addition, the present invention also provides a cell from a vascular plant, the cell transformed with a nucleotide sequence encoding a Na⁺ pumping ATPase.

Plants that are transformable with Agrobacterium tumefaciens include Arabidopsis, Barley, Potato, Tomato, Brassica, Cotton, Corn, Sunflower, Strawberries, Spinach, Lettuce, Wheat and Rice. Plants that are transformable by biolistic particle delivery systems (particle bombardment) include Soybean, Corn, Wheat, Rye, Barley, Atriplex, and Salicornia.

Methods and reagents for producing mature plants from cells are also known in the art, for example as described in Kumria et al. (2001) Plant Cell Tissue and Organ Culture 67: 63-71, and Przetakiewicz et al., (2003) Plant Cell Tissue and Organ Culture 73: 245-256.

As stated previously, the plant according to this embodiment of the present invention may be a plant in which all the cells in the plant express a Na⁺ pumping ATPase, or alternatively, be a plant in which a subset of the cells only express a Na⁺ pumping ATPase.

It will therefore be appreciated that in one embodiment the vascular plant is a transgenic plant in which all the cells of the plant have been transformed with a nucleotide sequence encoding a Na⁺ pumping ATPase.

In this case, driving the expression of the Na⁺ pumping ATPase from a constitutive promoter will result in transcription of the Na⁺ pumping ATPase in substantially all cell types in the plant.

However, driving the expression of the Na⁺ pumping ATPase with a cell type specific promoter will result only in transcription of the nucleotide sequence encoding the Na⁺ pumping ATPase in those tissues in which the cell type specific promoter is active. Driving the expression from an inducible promoter, such as a Na⁺-inducible promoter, will result in an induction of transcription in response to the inducing agent/treatment.

However, it should also be appreciated that the vascular plant according to the present invention may also be a chimeric plant in which only a subset of the cells that constitute the plant are transformed with a nucleotide sequence encoding a Na⁺ pumping ATPase. In a similar fashion to that described above, the nucleotide sequence may be expressed from a constitutive promoter, a cell type specific promoter or an inducible promoter. Methods for generating chimeric plants are known in the art.

In one embodiment, a plant cell expressing a Na⁺ pumping ATPase will have increased secretion of Na⁺, as compared to a similar cell that does not express a Na⁺ pumping ATPase. Thus, in one embodiment the level of expression of the Na⁺ pumping ATPase in the cell results in an increased secretion of Na⁺ from the cell, as compared to a similar cell not expressing a Na⁺ pumping ATPase. Methods of determining the ability of cells to secrete Na⁺ are known in the art, and include measurement of influx and efflux of ²²Na⁺, or by measurement of intracellular levels of Na⁺ using flame photometry.

Accordingly, the present invention also provides a method of increasing Na⁺ secretion from a cell from a vascular plant, the method including the step of expressing a Na⁺ pumping ATPase in the cell.

The present invention also provides a plant cell produced according to the method of this embodiment of the present invention.

Accordingly, the present invention also provides a cell from a vascular plant, the cell having increased secretion of Na⁺ due to the expression of a Na⁺ pumping ATPase in the cell.

The present invention also contemplates a plant (or a part of a plant) including one or more cells produced according to the method of this embodiment of the present invention. In addition, the present invention also contemplates a plant or a part of a plant propagated from the plant cells.

As described above, plants may be regenerated from the cells transformed with a nucleotide sequence encoding a Na⁺ pumping ATPase, thus producing a plant with cells having increased secretion of Na⁺.

Accordingly, in another embodiment the present invention provides a vascular plant including cells with increased Na⁺ secretion, the increased Na⁺ secretion due to the expression of a Na⁺ pumping ATPase in the cells.

In one embodiment, a plant cell expressing a Na⁺ pumping ATPase will have improved tolerance to Na⁺. Thus, in one embodiment the level of expression of the Na⁺ pumping ATPase in the cell results in the cell having an improved tolerance to Na⁺, as compared to a similar cell not expressing a Na⁺ pumping ATPase. In this regard, a variety of methods are known in the art for determining the tolerance of a plant cell to Na⁺, such as assessment of growth rates of the plant and the ability of the plant to maintain low shoot Na⁺ concentrations.

Accordingly, the present invention also provides a method of improving the Na⁺ tolerance of a cell from a vascular plant, the method including the step of expressing a Na⁺ pumping ATPase in the cells.

The present invention also includes a plant cell produced according to this method.

Accordingly, in another embodiment the present invention provides a cell from a vascular plant, the cell having improved tolerance to Na⁺ due to the expression of a Na⁺ pumping ATPase in the cell.

The present invention also contemplates a plant (or a part of a plant) including one or more cells produced according to the method of this embodiment of the present invention. In addition, the present invention also includes a plant or a part of a plant propagated from the plant cells.

As described above, plants may be regenerated from the cells transformed with a nucleotide sequence encoding a Na⁺ pumping ATPase, thus producing a plant with improved tolerance to Na⁺.

Accordingly, the present invention also includes a method of improving the Na⁺ tolerance of a vascular plant, the method including the step of expressing a Na⁺ pumping ATPase in cells of the plant.

In this regard, in one embodiment the method includes cloning or synthesizing a nucleic acid molecule encoding a Na⁺ pumping ATPase, inserting the nucleic acid molecule into a vector so that the nucleic acid molecule is operably linked to a promoter; inserting the vector into a plant cell or plant seed, and regenerating the plant from the plant cell or plant seed.

Thus, the present invention also provides a method of producing a vascular plant with improved tolerance to Na⁺, the method including the step of transforming a cell from a vascular plant with a nucleic acid encoding a Na⁺ pumping ATPase and producing a plant from the plant cell. Methods of regenerating plants from plant cells are known in the art.

Vascular plants expressing a Na⁺ pumping ATPase may be also crossed to other lines with desirable characteristics. For example, plants expressing a Na⁺ pumping ATPase may be crossed with plants that already have improved Na⁺ tolerance. Alternatively, the vascular plants expressing the Na⁺ pumping ATPase may be crossed with plants that are not Na⁺ tolerant, and plants that are Na⁺ tolerant selected.

It will also be appreciated that if the plants expressing the Na⁺ pumping ATPase are self-pollinated, homozygous progeny may be identified from the seeds of these plants. Plants grown from such seeds may show further improved Na⁺ tolerance over the parental line.

The present invention also provides a plant produced according to the method of this embodiment of the present invention.

Accordingly, in another embodiment the present invention provides a vascular plant with improved tolerance to Na⁺, the improved tolerance to Na⁺ being due to the expression of a Na⁺ pumping ATPase in cells of the plant.

The present invention also contemplates a plant, a plant cell or a part of a plant produced from such plants.

The present invention also provides a kit for transforming a cell from a vascular plant with a Na⁺ pumping ATPase, the kit including a nucleic acid encoding a Na⁺ pumping ATPase. In one embodiment, the kit further includes reagents and/or instructions for transforming plant cells.

As described above, the kit can be used to produce plants, or parts of plants, from the transformed cells. In addition, the kit can be used to produce plant cells, and plants including cells, with increased secretion of Na⁺. The kit can also be used to produce cells, and plants, with improved tolerance to Na⁺.

Finally, reference is made to standard textbooks of molecular biology that contain methods for carrying out basic techniques, encompassed by the present invention. See, for example, Maniatis et al., Molecular Cloning: A Laboratory Manual, Cold Spring Harbor Laboratory Press, New York (1982); Sambrook et al., Molecular Cloning: A Laboratory Manual, Cold Spring Harbor Laboratory Press, New York (1989); and Methods in Plant Molecular Biology, Maliga et al, Eds., Cold Spring Harbor Laboratory Press, New York (1995).

### DESCRIPTION OF SPECIFIC EMBODIMENTS

Reference will now be made to experiments that embody the above general principles of the present invention. However, it is to be understood that the following description is not to limit the generality of the above description.

### Example 1

### Cloning of Physcomitrella patens ENA1, ENA2 and Saccharomyces cerevisiae ENA1cDNAs

The full length ENA1 and ScENA1cDNAs in the cloning vectors pCR 2.1-TOPO (Invitrogen) and pJQ10 respectively, may be cloned as described in Benito, B., and Rodriguez-Navarro, A. (2003). The Plant Journal 36:382-389 and Benito et al. (1997) Biochimica et Biophysica Acta 1328(2):214-26. The cDNAs were obtained from Alonso Rodriguez-Navarro.

The nucleotide sequence of the Physcomitrella patens ENA1 cDNA is provided in GenBank Accession No. AJ564254, designated SEQ ID NO. 1. The cDNA encodes a 967 amino acid Na⁺ pumping ATPase, designated SEQ ID NO.2.

The nucleotide sequence of the Saccharomyces cerevisiae ENA1 cDNA is provided in GenBank Accession No. AJ564254, designated SEQ ID NO. 12. The cDNA encodes a 1091 amino acid Na⁺ pumping ATPase, designated SEQ ID NO. 13

Briefly, cDNAs representing the complete open reading frames of the PpENA1, PpENA2 and SCENA1 genes may be obtained by reverse transcription (RT)-PCR amplification. Total RNA extracted from Physcomitrella patens and Saccharomyces cerevisiae growing on media containing salt can be copied into cDNA and used as a template for PCR with gene specific primers. Overlapping cDNA fragments from RT-PCR can be combined acting as a template for the amplification of a full length cDNA. cDNAs can then be cloned into a commercially available cloning vector such as pCR2.1-TOPO (Invitrogen) or pGEM T-Easy (Promega). Alternatively restriction fragments of overlapping cDNAs may be ligated together at compatible sites to generate a full length cDNA.

Suitable primers are as follows:

(SEQ ID NO. 34)

| | |
|---|---|
| PpENA1-F | 5′ TCGTGACTGGGGAAGGGAAG 3′ |

(SEQ ID NO. 35)

| | |
|---|---|
| PpENA2-R | 5′ ACAGCATGGGTGCGGATTCT 3′ |

(SEQ ID NO. 18)

| | |
|---|---|
| PpENA2-F | 5′ ATGGTCGACATCCGAGAGTTGA 3′ |

(SEQ ID NO: 19)

| | |
|---|---|
| PpENA2-R | 5′ CAGGGTGGGAACTGGCACG 3′ |

(SEQ ID NO. 36)

| | |
|---|---|
| ScENA1-F | 5′ ATGGGCGAAGGAACTACTAAGG 3′ |

(SEQ ID NO. 37)

| | |
|---|---|
| ScENA1-R | 5′ ATTGTTTAATACCAATATTAACTTCTGTATGG 3′ |

PCR was performed in 25 μl reaction volumes using 500 ng of genomic DNA as template or 100 pg of cDNA. Elongase enzyme mix and buffer (Invitrogen) was used with 200 μM of each dNTP and 400 nm of primer. A preamplification step of 94° C. for 30s was conducted prior to 35 cycles of 94° C. for 30s, 55° C. for 30s, 68° C. for 3.5 min.

### Example 2

### Generating Mutant P. patens Lacking PpENA1 and/or PpENA2

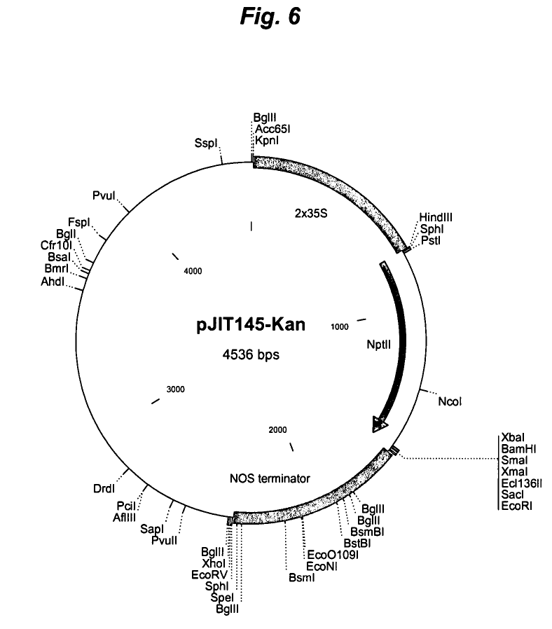

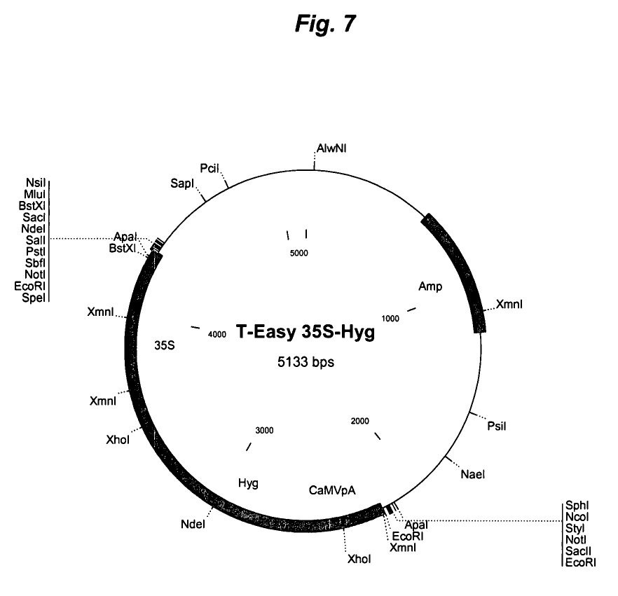

A restriction fragment of PpENA1 or PpENA2 may be transferred from cloned DNA into an appropriate vector e.g pGEM-T Easy (Promega). The knock-out cassette may then be generated by inserting a selective marker, e.g. a gene that confers resistance to kanamycin, hygromycin or basta, in the middle of the full length gene encoding PpENA1 or PpENA2. The cassette consists of sequence homologous to either PpENA1 or PpENA2 upstream or downstream the selective marker. Resistance to G-418 is obtained using the nptII gene behind the 35S-promoter from the pJIT145-Kan plasmid (FIG. 6). Resistance to hygromycin is obtained using the Hyg gene behind the 35S-promoter from the T-Easy 35S-Hyg plasmid (FIG. 7).

Mutant moss may then be generated by transformation. Protoplasts are generated by treating protonemal tissue with enzymes that remove the cell wall.

The protoplast is transformed (the knock-out cassette introduced) using a heat shock and PEG based method (Schaefer and Zrÿd (1997) Plant Journal 11(6): 1195-1206). Transformants may be selected by plating the protoplasts on a selective media (Schaefer and Zryd, 1997). Mutants lacking either PpENA1, PpENA2 or both may thus be generated.

### Example 3

### Generating Mutant P. patens Over Expressing PpENA1 and/or PpENA2

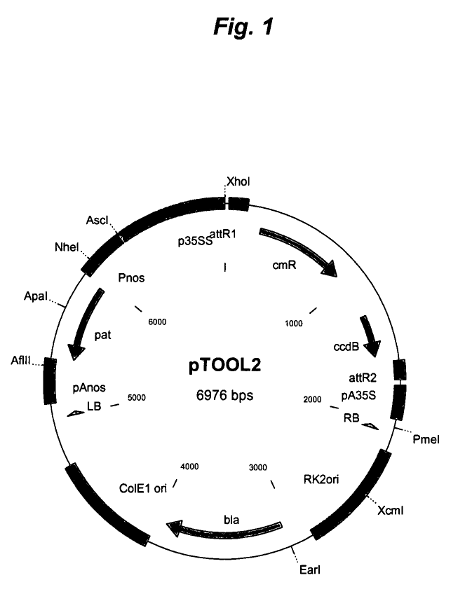

The full length clone of PpENA2 may be obtained by designing primers specific to the 5′ and 3′ end of the genomic sequence and performing PCR using cDNA as a template. A suitable over-expression vector is the pTOOL2 vector, as shown in FIG. 1. The construct may then be used to transform moss (as described above) and mutants over-expressing PpENA1, PpENA2 (or both) selected (as described above).

### Example 4

### Changes in Salt Tolerance

Wild type and mutant P. patens lacking or over expressing PpENA1, PpENA2 or both may be grown on media containing different levels of Na⁺ to test the differences in Na⁺ tolerance, as described in Benito and Rodriguez-Navarro (2003) The Plant Journal 36:382-389. Moss may be analysed for differences in visual phenotypes e.g. growth rate, ability to differentiate and generate gametophytes and for levels of necrosis. The intracellular level of Na⁺ may also be determined using flame photometry, as described in Essah et al., 2003: Plant Physiology 133, 307-318.

### Example 5

### Promoter Analysis of PpENA1 and PpENA2

The sequence of the native promoter of PpENA1 and PpENA2 may be determined by genomic walking, as described in Siebert et al. (1995) Nucleic Acids Research 23: 1087-1088, and analysed using appropriate search tools such as SignalScan for the presence of known regulatory elements (Higo, K., Ugawa Y., Iwamoto M. and T. Korenaga (1999). Nucleic Acids Research 27(1) 297-300).

The regulation in planta may also be determined by performing quantitative PCR and western blotting on tissue from wild type or mutant moss (described above) exposed to varying concentrations of Na⁺. Quantitative PCR may be performed as described in Jacobs et al (2003) Plant Cell 15: 2503-2513. Western blotting may be performed as described in Molecular Cloning: A Laboratory Manual. J. Sambrook, E. F. Fritsch, T. Maniatis 2nd ed. Cold Spring Harbor, N.Y.: Cold Spring Harbor Laboratory Press, 1989. The level of protein may be determined using polyclonal or monoclonal antibodies directed against unique parts of PpENA1 and PpENA2, as described in Example 13.

### Example 6

### Functional Analysis of PpENA1 and PpENA2

Based on structural information, truncated versions of PpENA1 and PpENA2 may be generated to alter the efficiency and/or regulation of the Na⁺-ATPase activity. For example, the first 255 amino acid residues of the amino-terminus may be removed from the PpENA1 protein by the amplification and subsequent expression of a truncated cDNA. Alternatively, the PpENA1 protein may be truncated at the COOH-terminus by the removal of the last 187 amino acid residues. Primers suitable for use in PCR for this purpose are as follows:

| | |
|---|---|
| PpENA1seqF5 | |
| 5′ CCTACATGCTCCTCGCATTT 3′ | (SEQ ID NO. 38) |
| PpENA1seqR10 | |
| 5′ TCACGGTGTTCCCGTGACGAGATTC 3′ | (SEQ ID NO. 39) |

These primers would be used in conjunction with the primers listed in Example 1 to generate truncated cDNAs of PpENA1.

Suitable PCR conditions are as described in Example 1, with cycling modified by reducing the extension time from 3.5 min to 2.5 min.

The functionality of the truncated versions may be tested by transforming mutant moss lacking PpENA1, PpENA2 (or both) and analysing for changes in the efficiency of Na⁺ exclusion and tolerance, as described above.

### Example 7

### Modification of PpENA and ScENA cDNAs

It has been found that the cloning of membrane transporters is facilitated by the growth of bacteria carrying plasmids with transporter genes at lower temperatures (30° C. or lower) for longer times (2 or more days) and selecting for colonies that are smaller and appear later on the plates.

Where the ENA cDNAs proved to be toxic to the bacterial cells used for molecular manipulations, a plant intron sequence from Arabidopsis was introduced into the coding region of the ENA cDNAs by PCR. Outside of plants this leads to the transcription of a non functional Na⁺-ATPase with a modified tertiary structure. This greatly enhances the efficiency of engineering vectors for subsequent genetic manipulations. The small Arabidopsis WRKY33 first intron (286 bp) was used for this purpose, the DNA sequence (designated SEQ ID NO. 40) being as follows:

| | |
|---|---|
| 5′ TCCTCCTCTGCTAACGTAAGCCTCTCTGTTTTTTTTCTCTGTTTCTTTTGAAATGAATCCAA | (SEQ ID NO. 40) |
| TTAGTGATGATAATCTGTGTTTGATGTATCATTGATTTAACATCTTGACAATGAATCGTGATCG | |
| GAAGTGATAAAGTTATGGGTCAACGGTTTCAAAGAGAGAGAAAGACTTTTAGAGTCAACTCTCG | |
| ACTCTTTCTTAATTATGTTATTGCTATTTGTCTCTTTTCTTGAAGTCTGAACAATTCTTGGGAT | |
| TGTTTTGCAGGTTCTAGCTTCTCCAACCACAG 3′ | |

The WRKY33 1st intron may be PCR amplified using the following oligonucleotides:

| | |
|---|---|
| WRKY33F | |
| 5′ TCCTCCTCTGCTAACGTAAGCC 3′ | (SEQ ID NO: 20) |
| WRKY33R | |
| 5′ CTGTGGTTGGAGAAGCTAGAACC 3′ | (SEQ ID NO: 21) |

PCR was performed in 25 μl reaction volumes using 50 pg of plasmid DNA as template. Elongase enzyme mix and buffer (Invitrogen) was used with 200 μm of each dNTP and 400 nm of primers. A preamplification step of 94° C. for 30 s was conducted prior to 35 cycles of 94° C. for 30 s, 55° C. for 30 s, 68° C. for 20 sec.

The intron was inserted into the ENA sequence using a series of PCR steps. Initially the PpENA1 sequence was amplified in two fragments with the junction being positioned such that intron splice rules would be met when the WRKY intron was inserted. The WRKY intron was amplified using a set of oligonucleotides with ˜20 bp overhangs at the 5′ ends that corresponded to the sequence of the PpENA1cDNA sequences at the junction point. The purified PCR products are then pooled and PCR was performed in the absence of oligonucleotides. The WRKY PCR product hybridised to the two PpENA1 sequences by means of the complimentary sequence at both ends and acting as a primer for DNA extension by the polymerase. Oligonucleotides designed to amplify the full PpENA1 sequence were introduced into the PCR after 5 cycles and the modified PpENA1 sequence containing the intron was thus produced.

### Example 8

### Comparison of Moss and Higher Plant Codon Usage

An analysis of the codon usage of Physcomitrella patens and 10 higher plants, encompassing the transformation recipients described herein, was determined using the web-based codon usage database (Nakamura, Y., Gojobori, T. and Ikemura, T. (2000) Nucleic Acids Res. 28: 292.), located at http://www.kazusa.or.ip/codon/. The database details are shown in Table 1. This analysis utilised all of the full-length coding sequences of each species currently available (as of May 8, 2004) in the GenBank database.

TABLE 1

| | Species | Number of CDS | Number of codons |
|---|---|---|---|
| Pp | Physcomitrella patens | 188 | 83355 |
| Hv | Hordeum vulgare | 600 | 226396 |
| Os | Oryza sativa | 41580 | 16166879 |
| Sb | Sorghum bicolor | 293 | 156922 |
| Ta | Triticum aestivum | 799 | 296870 |
| Zm | Zea mays | 1697 | 728757 |
| Ath | Arabidopsis thaliana | 65605 | 26209750 |
| Gh | Gossypium hirsutum | 286 | 99990 |
| Gm | Glycine max | 822 | 337325 |
| Le | Lycopersicon esculentum | 1067 | 466408 |
| Nt | Nicotiana tabacum | 1159 | 434493 |

(The species abbreviations used above are used in subsequent tables)

This analysis demonstrated that the codon usage of the cereals analysed differed considerably from Physcomitrella patens by having a stronger preference for G or C in the third base position of codons, as shown in Table 2.

The most significant differences between Physcomitrella patens and cereals were found in the codon preference for cysteine, phenylalanine, glycine, serine and threonine (as highlighted in Table 2).

TABLE 2
Comparison of moss and cereal codon usage

| | | | Frequency of codon use | | | | | |
|---|---|---|---|---|---|---|---|---|
| Amino acid | | Codon | Pp | Hv | Os | Sb | Ta | Zm |
| A | Alanine | GCU | 0.30 | 0.19 | 0.21 | 0.24 | 0.20 | 0.24 |
| | | GCC | 0.21 | 0.39 | 0.32 | 0.32 | 0.38 | 0.34 |
| | | GCA | 0.26 | 0.16 | 0.24 | 0.21 | 0.18 | 0.18 |
| | | GCG | 0.23 | 0.26 | 0.24 | 0.23 | 0.24 | 0.24 |
| C | Cysteine | UGU | 0.41 | 0.27 | 0.34 | 0.37 | 0.29 | 0.32 |
| | | UGC | 0.59 | 0.73 | 0.66 | 0.63 | 0.71 | 0.68 |
| D | Aspartic acid | GAU | 0.54 | 0.36 | 0.48 | 0.46 | 0.37 | 0.42 |
| | | GAC | 0.46 | 0.64 | 0.52 | 0.54 | 0.63 | 0.58 |
| E | Glutamic acid | GAA | 0.38 | 0.27 | 0.37 | 0.36 | 0.29 | 0.33 |
| | | GAG | 0.62 | 0.73 | 0.63 | 0.64 | 0.71 | 0.67 |
| F | Phenylalanine | UUU | 0.43 | 0.28 | 0.38 | 0.37 | 0.34 | 0.34 |
| | | UUC | 0.57 | 0.72 | 0.62 | 0.63 | 0.66 | 0.66 |
| G | Glycine | GGU | 0.26 | 0.17 | 0.20 | 0.21 | 0.18 | 0.20 |
| | | GGC | 0.23 | 0.44 | 0.37 | 0.37 | 0.39 | 0.41 |
| | | GGA | 0.30 | 0.17 | 0.21 | 0.21 | 0.20 | 0.18 |
| | | GGG | 0.21 | 0.22 | 0.22 | 0.21 | 0.23 | 0.21 |
| H | Histidine | CAU | 0.49 | 0.36 | 0.45 | 0.45 | 0.40 | 0.41 |
| | | CAC | 0.51 | 0.64 | 0.55 | 0.55 | 0.60 | 0.59 |
| I | Isoleucine | AUU | 0.43 | 0.27 | 0.34 | 0.33 | 0.28 | 0.31 |
| | | AUC | 0.40 | 0.57 | 0.46 | 0.46 | 0.56 | 0.51 |
| | | AUA | 0.17 | 0.16 | 0.21 | 0.21 | 0.15 | 0.18 |
| K | Lysine | AAA | 0.35 | 0.22 | 0.34 | 0.32 | 0.21 | 0.27 |
| | | AAG | 0.65 | 0.78 | 0.66 | 0.68 | 0.79 | 0.73 |
| L | Leucine | UUA | 0.09 | 0.04 | 0.07 | 0.07 | 0.04 | 0.06 |
| | | UUG | 0.27 | 0.13 | 0.17 | 0.17 | 0.14 | 0.14 |
| | | CUU | 0.18 | 0.16 | 0.17 | 0.18 | 0.16 | 0.17 |
| | | CUC | 0.14 | 0.34 | 0.28 | 0.25 | 0.31 | 0.27 |
| | | CUA | 0.08 | 0.07 | 0.09 | 0.09 | 0.08 | 0.08 |
| | | CUG | 0.24 | 0.27 | 0.23 | 0.25 | 0.26 | 0.27 |
| N | Asparagine | AAU | 0.48 | 0.32 | 0.45 | 0.43 | 0.34 | 0.37 |
| | | AAC | 0.52 | 0.68 | 0.55 | 0.57 | 0.66 | 0.63 |
| P | Proline | CCU | 0.33 | 0.21 | 0.24 | 0.24 | 0.18 | 0.23 |
| | | CCC | 0.23 | 0.27 | 0.21 | 0.22 | 0.22 | 0.25 |
| | | CCA | 0.25 | 0.22 | 0.25 | 0.29 | 0.35 | 0.25 |
| | | CCG | 0.19 | 0.30 | 0.31 | 0.25 | 0.24 | 0.27 |
| Q | Glutamine | CAA | 0.43 | 0.29 | 0.41 | 0.40 | 0.52 | 0.37 |
| | | CAG | 0.57 | 0.71 | 0.59 | 0.60 | 0.48 | 0.63 |
| R | Arginine | CGU | 0.13 | 0.11 | 0.11 | 0.12 | 0.11 | 0.11 |
| | | CGC | 0.13 | 0.26 | 0.23 | 0.21 | 0.26 | 0.25 |
| | | CGA | 0.17 | 0.06 | 0.10 | 0.09 | 0.06 | 0.08 |
| | | CGG | 0.17 | 0.19 | 0.19 | 0.18 | 0.16 | 0.16 |
| | | AGA | 0.18 | 0.12 | 0.15 | 0.17 | 0.14 | 0.15 |
| | | AGG | 0.21 | 0.26 | 0.22 | 0.24 | 0.26 | 0.26 |
| S | Serine | UCU | 0.20 | 0.14 | 0.16 | 0.16 | 0.16 | 0.17 |
| | | UCC | 0.14 | 0.26 | 0.21 | 0.22 | 0.24 | 0.22 |
| | | UCA | 0.15 | 0.13 | 0.16 | 0.16 | 0.16 | 0.15 |
| | | UCG | 0.17 | 0.14 | 0.16 | 0.14 | 0.13 | 0.14 |
| | | AGU | 0.16 | 0.09 | 0.11 | 0.11 | 0.09 | 0.11 |
| | | AGC | 0.18 | 0.25 | 0.20 | 0.21 | 0.23 | 0.22 |
| T | Threonine | ACU | 0.32 | 0.17 | 0.22 | 0.22 | 0.21 | 0.23 |
| | | ACC | 0.21 | 0.42 | 0.31 | 0.32 | 0.40 | 0.34 |
| | | ACA | 0.23 | 0.18 | 0.24 | 0.25 | 0.19 | 0.21 |
| | | ACG | 0.23 | 0.23 | 0.24 | 0.21 | 0.19 | 0.22 |
| V | Valine | GUU | 0.26 | 0.19 | 0.23 | 0.23 | 0.22 | 0.23 |
| | | GUC | 0.18 | 0.34 | 0.30 | 0.31 | 0.33 | 0.31 |
| | | GUA | 0.13 | 0.08 | 0.11 | 0.11 | 0.09 | 0.09 |
| | | GUG | 0.43 | 0.39 | 0.36 | 0.35 | 0.37 | 0.37 |
| Y | Tyrosine | UAU | 0.37 | 0.27 | 0.41 | 0.37 | 0.31 | 0.32 |
| | | UAC | 0.63 | 0.73 | 0.59 | 0.63 | 0.69 | 0.68 |

In contrast to the cereals, there was found to be little difference between Physcomitrella patens and the dicot species chosen in the nucleotide preference for the third-base position of codons, as shown in Table 3. However, differences were found in the codon preference for aspartic acid, arginine and valine (highlighted in the table).

TABLE 3
Comparison of moss and dicot codon usage

| | | | Frequency of codon use | | | | | |
|---|---|---|---|---|---|---|---|---|
| Amino acid | | Codon | Pp | Ath | Gh | Gm | Le | Nt |
| A | Alanine | GCU | 0.30 | 0.44 | 0.44 | 0.39 | 0.45 | 0.44 |
| | | GCC | 0.21 | 0.16 | 0.23 | 0.23 | 0.15 | 0.17 |
| | | GCA | 0.26 | 0.27 | 0.26 | 0.30 | 0.32 | 0.31 |
| | | GCG | 0.23 | 0.14 | 0.08 | 0.08 | 0.08 | 0.08 |
| C | Cysteine | UGU | 0.41 | 0.60 | 0.50 | 0.50 | 0.61 | 0.57 |
| | | UGC | 0.59 | 0.40 | 0.50 | 0.50 | 0.39 | 0.43 |
| D | Aspartic acid | GAU | 0.54 | 0.68 | 0.67 | 0.62 | 0.72 | 0.68 |
| | | GAC | 0.46 | 0.32 | 0.33 | 0.38 | 0.28 | 0.32 |
| E | Glutamic acid | GAA | 0.38 | 0.52 | 0.52 | 0.50 | 0.56 | 0.54 |
| | | GAG | 0.62 | 0.48 | 0.48 | 0.50 | 0.44 | 0.46 |
| F | Phenylalanine | UUU | 0.43 | 0.51 | 0.47 | 0.50 | 0.59 | 0.58 |
| | | UUC | 0.57 | 0.49 | 0.53 | 0.50 | 0.41 | 0.42 |
| G | Glycine | GGU | 0.26 | 0.34 | 0.35 | 0.30 | 0.35 | 0.34 |
| | | GGC | 0.23 | 0.14 | 0.17 | 0.19 | 0.14 | 0.17 |
| | | GGA | 0.30 | 0.37 | 0.30 | 0.32 | 0.36 | 0.34 |
| | | GGG | 0.21 | 0.15 | 0.18 | 0.18 | 0.15 | 0.15 |
| H | Histidine | CAU | 0.49 | 0.61 | 0.60 | 0.55 | 0.67 | 0.61 |
| | | CAC | 0.51 | 0.39 | 0.40 | 0.45 | 0.33 | 0.39 |
| I | Isoleucine | AUU | 0.43 | 0.41 | 0.45 | 0.47 | 0.50 | 0.50 |
| | | AUC | 0.40 | 0.35 | 0.35 | 0.30 | 0.25 | 0.25 |
| | | AUA | 0.17 | 0.24 | 0.20 | 0.23 | 0.25 | 0.25 |
| K | Lysine | AAA | 0.35 | 0.48 | 0.43 | 0.42 | 0.50 | 0.49 |
| | | AAG | 0.65 | 0.52 | 0.57 | 0.58 | 0.50 | 0.51 |
| L | Leucine | UUA | 0.09 | 0.14 | 0.11 | 0.10 | 0.15 | 0.14 |
| | | UUG | 0.27 | 0.22 | 0.24 | 0.24 | 0.26 | 0.24 |
| | | CUU | 0.18 | 0.26 | 0.28 | 0.26 | 0.26 | 0.26 |
| | | CUC | 0.14 | 0.17 | 0.17 | 0.18 | 0.12 | 0.14 |
| | | CUA | 0.08 | 0.11 | 0.08 | 0.09 | 0.10 | 0.10 |
| | | CUG | 0.24 | 0.11 | 0.12 | 0.13 | 0.11 | 0.12 |
| N | Asparagine | AAU | 0.48 | 0.52 | 0.50 | 0.49 | 0.63 | 0.60 |
| | | AAC | 0.52 | 0.48 | 0.50 | 0.51 | 0.37 | 0.40 |
| P | Proline | CCU | 0.33 | 0.38 | 0.39 | 0.36 | 0.39 | 0.37 |
| | | CCC | 0.23 | 0.11 | 0.17 | 0.19 | 0.12 | 0.13 |
| | | CCA | 0.25 | 0.33 | 0.34 | 0.37 | 0.40 | 0.40 |
| | | CCG | 0.19 | 0.18 | 0.10 | 0.08 | 0.09 | 0.10 |
| Q | Glutamine | CAA | 0.43 | 0.56 | 0.57 | 0.55 | 0.61 | 0.58 |
| | | CAG | 0.57 | 0.44 | 0.43 | 0.45 | 0.39 | 0.42 |
| R | Arginine | CGU | 0.13 | 0.17 | 0.17 | 0.14 | 0.15 | 0.16 |
| | | CGC | 0.13 | 0.07 | 0.08 | 0.13 | 0.07 | 0.08 |
| | | CGA | 0.17 | 0.12 | 0.12 | 0.08 | 0.11 | 0.11 |
| | | CGG | 0.17 | 0.09 | 0.09 | 0.06 | 0.06 | 0.08 |
| | | AGA | 0.18 | 0.35 | 0.28 | 0.31 | 0.35 | 0.32 |
| | | AGG | 0.21 | 0.20 | 0.26 | 0.28 | 0.26 | 0.26 |
| S | Serine | UCU | 0.20 | 0.28 | 0.22 | 0.24 | 0.26 | 0.26 |
| | | UCC | 0.14 | 0.13 | 0.17 | 0.17 | 0.12 | 0.14 |
| | | UCA | 0.15 | 0.20 | 0.20 | 0.20 | 0.25 | 0.23 |
| | | UCG | 0.17 | 0.10 | 0.09 | 0.06 | 0.07 | 0.07 |
| | | AGU | 0.16 | 0.16 | 0.16 | 0.17 | 0.18 | 0.17 |
| | | AGC | 0.18 | 0.13 | 0.16 | 0.15 | 0.11 | 0.13 |
| T | Threonine | ACU | 0.32 | 0.34 | 0.35 | 0.34 | 0.39 | 0.39 |
| | | ACC | 0.21 | 0.20 | 0.27 | 0.28 | 0.17 | 0.19 |
| | | ACA | 0.23 | 0.30 | 0.28 | 0.30 | 0.35 | 0.33 |
| | | ACG | 0.23 | 0.15 | 0.10 | 0.08 | 0.09 | 0.09 |
| V | Valine | GUU | 0.26 | 0.41 | 0.42 | 0.39 | 0.43 | 0.41 |
| | | GUC | 0.18 | 0.19 | 0.20 | 0.18 | 0.15 | 0.17 |
| | | GUA | 0.13 | 0.15 | 0.12 | 0.11 | 0.17 | 0.17 |
| | | GUG | 0.43 | 0.26 | 0.26 | 0.32 | 0.25 | 0.25 |
| Y | Tyrosine | UAU | 0.37 | 0.51 | 0.55 | 0.52 | 0.59 | 0.57 |
| | | UAC | 0.63 | 0.49 | 0.45 | 0.48 | 0.41 | 0.43 |

In the case where differences in codon usage between moss and higher plants are found to significantly reduce the level of PpENA1 expression in the recipient plant, “recursive-PCR” (as described in Prodromou C, Pearl LH., (1992) Protein Eng. 5: 827-9.) may be used to modify the codon usage of PpENA1 to mirror that of the recipient plant.

### Example 9

### Isolation of the PIP2.2 and VHA-c3 Promoters and Their Use to Drive ENA Expression

The PIP2.2 and VHA-c3 promoters may be used to drive expression of the Na⁺ ATPase. These promoters may be cloned and used to drive the NaCl-dependant expression of PpENA1 in the binary vector(s) named herein.

The MIPS Accession numbers for PIP2.2 and VHA-c3 are At2g37180 and At4g38920, respectively.

The following primers may be used to amplify 2013 bp of the PIP2.2 promoter sequence corresponding to positions 57-2051 bp upstream of the PIP2.2 translation initiation site.

(SEQ ID NO. 22)

| | |
|---|---|
| PIP2For | 5′ TTTTTCGGTGTAAGCTGAGTG 3′ |

(SEQ ID NO. 23)

| | |
|---|---|
| PIP2Rev | 5′ ATCGAAAAACGGAGTTGGTG 3′ |

(SEQ ID NO. 24)

| | |
|---|---|
| PIP2F1 | 5′ AAGGCGCGCCTCTGTCATAGGACACTACAATCAAA 3′ |

(SEQ ID NO.25)

| | |
|---|---|
| PIP2R1 | 5′ AAACGCGTTGTTTGTGAAGACTGAAGAGACG 3′ |

The PIP2.2 promoter sequence may be PCR amplified from Arabidopsis thaliana genomic DNA using the forward primer PIP2For (SEQ ID NO. 22) and the reverse primer PIP2Rev (SEQ ID NO. 23). The product of this reaction can then be used as a template for a second round of PCR using the forward primer PIP2 μl (SEQ ID NO. 24) and the reverse primer PIP2R1 (SEQ ID NO. 25). The second round of PCR introduces the restriction sites Ascl (5′) and Mlul (3′) to the promoter sequence enabling the later cloning into plant transformation vectors. Second round PCR products were cloned into the pGemT cloning vector (Promega) and sequenced.

First round PCR was performed in 25 μl using 100 ng of genomic DNA as template. Second round PCR was performed in 25 μl using 50 pg of purified first round product as template. Elongase enzyme mix and buffer (Invitrogen) was used with 200 μm of each dNTP and 400 nm of primer. A preamplification step of 94° C. for 2 min was conducted prior to 5 cycles of 94° C. for 1 min, 58° C. for 1 min, 72° C. for 2.5 min, followed by 5 cycles of 94° C. for 1 min, 56° C. for 1 min, 72° C. for 2.5 min and 20 cycles of 94° C. for 1 min, 54° C. for 1 min, 72° C. for 2.5 min.

The PIP2.2 promoter sequence (designated SEQ ID No. 41) is as follows:

[symbol]

TTAAATCTGTCATAGGACACTACAATCAAATTATAACTCTATATATACT
CCATCGACTATATATTGACTCAACATATCATGATAGAATTACATATGAGTCAGATGTAATTTTATGATCT
GTTATTGTGGATTCATTGTTAGCAAGCATATATATACGTGTGTTCAGAAGACTAAAAAATTTACTATGAA
ATGAAAGAACATTTATTTGTTTGGAACAAAAAATGTTTATCATAACAAATTAACCCATTTTTCCACAGAG
AACAATACCATTCGTAACCAATATCGAGTAGACAATTCTTTAACAAAACAGAAAGCGACAAAGATGAAAG
AAAACAAAAATAAAACCAGGGGAGAGACCGAGAGAGAGAGAGCACCTTTCTGACGTGGAAATATAATCCA
ATAAGAGCAGAGAAATGTCGCTACAACGATAAGGATATTGTGACGTGGCAAAACATTGGCCATGAACACC
ACATTACACCACATTCCTTTTGTCTATGTACACTTTTTATTTTTCCAATTTATTTTTGTAGACAGACTAG
TGATTAGTGAATACTGAATAATATACGTAAGAAAATTGCAATTGGAAATTTGGAATTGAGGAGTGAGAGC
AAATGATTGTTGAATATGGGGACTAAACAACGTGGCATAAGAGGAGTGGTTGGGACGGCTGAACTGGAGT
TGGACTTAATCTGTATGGACGGTGCCGATGCAATTGACGGAGCTAATCAATTCTATATGGGGCGGTTTCT
CCGGTCCAGTGGACCCAACTTTCATCATATTTTCTACTTTAGTGGAAACATAACCCGTGAAGCGACGCCG
TTTCTTTTATCATGTCCATGTGATAAATTATGTTTTTGTTATATGGTAGGGTTAGCTGAGAGCTATCAAA
AGACTCTTTTTTATCCACCTAATAGATTTGATTTGTAACGTTAAGAGCATAGGAAGTCAATTTAATCGTT
ACTAATTACATGCATAAGAACTAGTATAACTATATAAGGAGCTCCTTCTAGAAATTAAATGAAGGATGAT
AGAATCTAGATAATCAGAAATTTTACTATTGATCAATCTAGCTATCTCGTAGTTCAAAAAGCTTTATCGT
TAACAAGTAACAACTTTCAAGTATTGCCCAAATAGATAAGGTTCATAACTTCATATTTTTTATTTATTTT
ATGTGTAAAAGAGTGACAGTCTATATTATTCTAGGGGGAGGACAAGGCTCATGACATAGGACAAGAGAAA
GAAAAATATAGAAGCATATAGTATATTAGGGTCGGTCCAAATGAAAACAACGTTTAGGTATGGGGCGGCG
AGGCTAAGTTAAATTAACCACAAAACTCCATTATCAACCATAATTTTAGAATTAAAAGGTCTCTGTTCCT
ATTGATAGCTCCACAATCATTCTTTTAAATAATCAGAATCTCAAATAAGTTCATCTTTAGTTACAGATTT
GTATCAATAGTTGAAGTTGAAACCAAAATAATAATAATTTAGTTATAGTTAATTTTGTCAACAAAACAAT
ACCTTAACTATCATATTATGACAAACACTAATTGAGATGAAAAACTCTTAGCAGTAGCTAATTCTTACTA
TCATCAGTTAATTATACTAATGTATATGGAAATTCTGCTTAAACAAAAAAAAAACAGTGGAACATGAATA
TATTAAGCAAAATCAGTTTCTATTGATTATGTAGCAATGATTAGATTGGTTTAGATTATATATCATCATG
ACAGCTAGCTAGGTAATTAATTAGTGAAAGAAAGTTTCCACAAAAATAATCATAATCGTCATACACACAA
TTCTATATTCATTTCATTGAAACGAATAATAAAAACAACCATAAGCCTACCAAAAGGAAAACATTATCGT
AATATAATCAATCAATAACACGTATACAATTATTAACGTATATTGACAAGCAAAATTAATGAGAGCACTC
ACTATAGCTATAGTCTCTCTATATAAACAACTTTCATTCGTCTCTTCAGTCTTCACAAACACAACATATC
CACAATACAAAACACAACTTTCATATATAACAAAAAAAGTTATAGAAATGGCCAAAGACGTGGAAGGACC
TGAGGGATTTCAGACAAGAGACTACGAAGATCCGC

[symbol]

The PIP2For and PIP2Rev primer sites are in bold italics, the PIP2.2 translation initiation codon is indicated by underlining and the PIP2F1 and PIP2R1 primer sites are in bold and underlined.

The following primers may be used to amplify 817 bp of the Arabidopsis VHA-c3 promoter sequence corresponding to positions 3-819 bp upstream of the VHA-c3 translation initiation site.

(SEQ ID NO. 26)

| | |
|---|---|
| VHAc3For | 5′ TGCTTACCACAGATTGTGTTCC 3′ |

(SEQ ID NO. 27)

| | |
|---|---|
| VHAc3Rev | 5′ AAGGAAGCCGAAGAAAGGAG 3′ |

(SEQ ID NO. 28)

| | |
|---|---|
| VHAc3F1 | 5′ AAGGCGCGCCTCCAAATCATAAGCAGTTCCAT 3′ |

(SEQ ID NO. 29)

| | |
|---|---|
| VHAc3R2 | 5′ AAACGCGTCTCAGGCGATTCTGGATCTT 3′ |

The VHA-c3 promoter sequence may be PCR amplified from Arabidopsis thaliana genomic DNA using the forward primer VHAc3For (SEQ ID NO. 26) and the reverse primer VHAc3Rev (SEQ ID NO. 27). The product of this reaction can then be used as a template for a second round of PCR using the forward primer VHAc3 μl (SEQ ID NO. 28) and the reverse primer VHAc3R1 (SEQ ID NO. 29). The second round of PCR introduces the restriction sites Ascl (5′) and Mlul (3′) to the promoter sequence enabling the later cloning into plant transformation vectors. Second round PCR products were cloned into the pGem T-Easy cloning vector (Promega) and sequenced.

First round PCR was performed in 25 μl using 100 ng of geomic DNA as template. Second round PCR was performed in 25 μl using 50 pg of purified first round product as template. Elongase enzyme mix and buffer (Invitrogen) was used with 200 μm of each dNTP and 400 nm of primer. A preamplification step of 94° C. for 2 min was conducted prior to 5 cycles of 94° C. for 1 min, 56° C. for 1 min, 72° C. for 1 min, followed by 5 cycles of 94° C. for 1 min, 54° C. for 1 min, 72° C. for 1 min and 20 cycles of 94° C. for 1 min, 50° C. for 1 min, 72° C. for 1 min.

The VHA-c3 promoter sequence (designated SEQ ID NO. 42) is as follows:

[symbol]

TTTGTAGTAATCGGGCTTGTAGCGCCCATTTTCATACTGCCCACCACT
CTCCATCCTCTTACTTTCAACTGCAATGGAGAAATTGATATCAAACATTGTGAAACTAGGCTGACGAGTA
ACTAAAAACAGAAATACTCCAAATCATAAGCAGTTCCATAACATACATTTAACCCAAATAAATCGAGAAA
TCGTATCATATCCCACAAGTCAGCGTAATACCATCCAAACCAAACGATGAAGAAAACAATGGAGCAAGTA
AGATACGCGGGAACATATATAGAGTTCGAATTTCAAGTTAAAGCAACGACGAGAGAGCTCCCAGAAGAAC
CAAAATTCGAAGAAAATGAAAATTGTAGAGAGAAAAACTTGGCATGCTGAAATTAACAGATAGGTCAAGA
ACACGATTAACGATCGAAGACTTACGGATTTCAACGAGCCTTCAGGAGAAACAAGCAACGGAAATCGAGA
AAGATCTGAGGATACTTGGAAATGGTGTCTGTGTAATGTGGCAAGAAGTGGAAGACGAGCCAGGTACTCT
CGGTTCAATTTACTAATATACCCTTGTCTTAAAACTGCTAAACGAGAGCAAGCAAGAAGGTTATTATTGT
CTATCCATCTTACTCGTAAAAATGCAAAGACGTTTCTGTTTCAATCTCTCCAAATATAAGCCAAACAGGA
TATGATTTTGGTTCTGGTGGATCATTCTAGTGGGCCGTATGATGGGCCTAAGAATAAGGCAACTAATCTG
GGTCGAATACGGGTAGACCCGGGTTGAGATCCCGACGTGTGCGCTTCGCTGTTGTAGTAGTAGTATATCT
CATCATCAATCAGGCTTTTGAGCCTCGGAAACTCAATCCTTGTATATTCAACGGAGAGAGATCTGCGAGA
GAAAGAGAGATCAGATTCCGGTGTTCCAAGGAAGCACATATTTTAAGATCCAGAATCGCCTGAGAGATGT
CTACCTTCAGTGGCGATGAGACCG

[symbol]

The VHAc3For and VHAc3Rev primer sites are in bold italics, the VHA-c3 translation initiation codon is underlined and the VHAc3F1 and VHAc3R1 primer sites are in bold and underlined.

### Example 10

### Construction of Plant Transformation Vectors

The PpENA1cDNA may be PCR amplified from the pCR 2.1 TOPO cloning vector using the forward primer PpENA1GF: 5′-GGG GAC MG TTT GTA CM AAA AGC AGG CTT GAT GGA GGG CTC TGG GGA CM G-3′ (SEQ ID NO. 30) and the reverse primer PpENA1 GR: 5′-GGG GAC CAC TTT GTA CM GM AGC TGG GTA TCA CAT GTT GTA GGG AGT TTT MT G-3′ (SEQ ID NO. 31) which introduces Gateway® recombination signal sequences distal to the PpENA1DNA sequence.

PCR was performed in 25 μl using 50 pg of plasmid DNA as template. Elongase enzyme mix and buffer (Invitrogen) was used with 200 μm of each dNTP and 400 nm of primer. A preamplification step of 94° C. for 30 s was conducted prior to 35 cycles of 94° C. for 30 s, 55° C. for 30 s, 68° C. for 3.5 min.

The resultant PCR fragment may be recombined using Gateway® technology into the pTOOL2 binary vector (FIG. 1) via pDONR201 (Invitrogen). (Arabidopsis-35S constitutive expression, BASTA resistance). Briefly, the Gateway® Technology is a universal cloning method based on the site-specific recombination properties of bacteriophage lambda (as described in Landy (1989) Ann. Rev. Biochem. 58, 913-949). The Gateway® Technology provides a rapid and highly efficient way to move DNA sequences into multiple vector systems for functional analysis and protein expression. A full description of the technology may be found at the following site: http://www.invitrogen.com/content/sfs/manuals/gatewayman.pdf

The SCENA1cDNA may be PCR amplified from the pJQ10 vector using the forward primer ScENA1GF: 5′-GGG GAC MG TTT GTA CM AAA AGC AGG CTT ATG GGC GM GGA ACT ACT MG GA-3′ (SEQ ID NO. 32) and the reverse primer ScENA1 GR: 5′-GGG GAC CAC TTT GTA CM GM AGC TGG GTT TCA TTG TTT MT ACC MT ATT MC TT-3′ (SEQ ID NO. 33) which introduced Gateway® (Invitrogen) recombination signal sequences distal to the ScENA1DNA sequence.

Suitable PCR conditions are as follows: PCR may be performed in 25 μl using 50 pg of plasmid DNA as template. Elongase enzyme mix and buffer (Invitrogen) was used with 200 μm of each dNTP and 400 nm of primer. A preamplification step of 94° C. for 30 s was conducted prior to 35 cycles of 94° C. for 30 s, 55° C. for 30 s, 68° C. for 3.5 min.

The resultant PCR fragment may be recombined into the pTOOL2 binary vector via pDONR201 (Invitrogen). (Arabidopsis-35S constitutive expression, BASTA resistance).

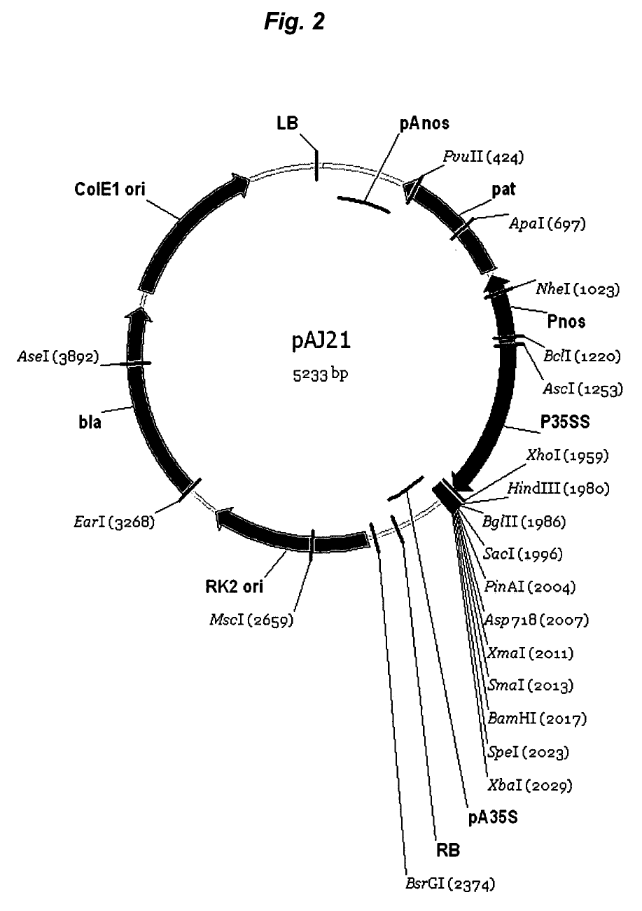

The PpENA1, SCENA1cDNAs and modified versions of same may be cut from their respective cloning vectors and introduced into the complementary sites of the pAJ21 binary plasmid (FIG. 2) (Arabidopsis-35S constitutive expression, BASTA resistance).

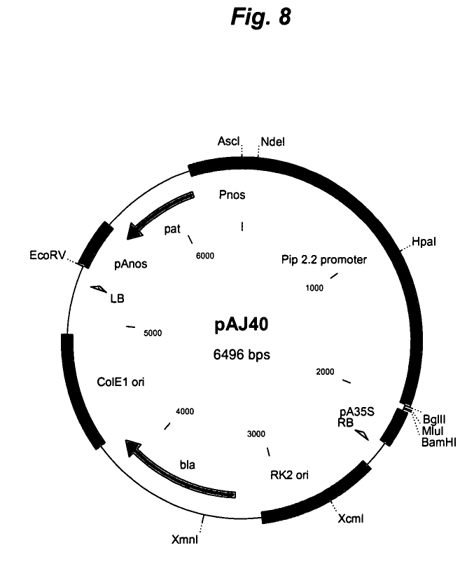

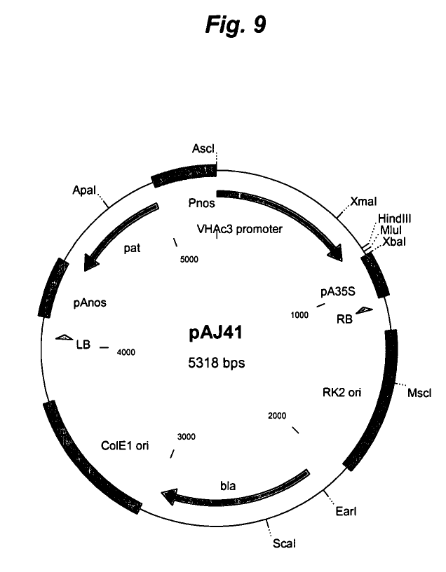

The PpENA1, ScENA1cDNAs and modified versions of same may be cut from their respective cloning vectors and introduced into the complementary sites of the pAJ40 and pAJ41 binary plasmids (Arabidopsis—salt stress induced expression, BASTA resistance). The resultant plasmid pAJ40-Pip2.2 is shown in FIG. 8 and plasmid pAJ41-VHAc2 is shown in FIG. 9.

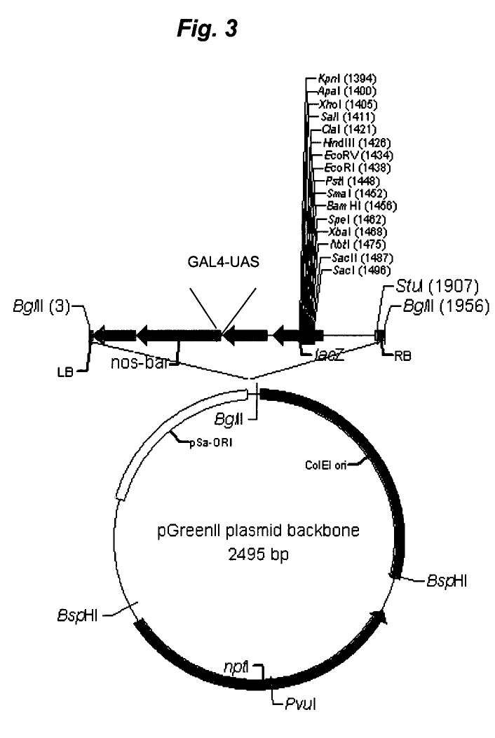

The PpENA1, ScENA1cDNAs and modified versions of same may be cut from their respective cloning vectors and introduced into the complementary sites of the pGreenII0229UAS+Nos5A binary plasmid (FIG. 3; Arabidopsis—GAL4 UAS activation tagged lines, expressing GAL4 contain nptII giving kanamycin resistance). The second round of selection is done using Basta.

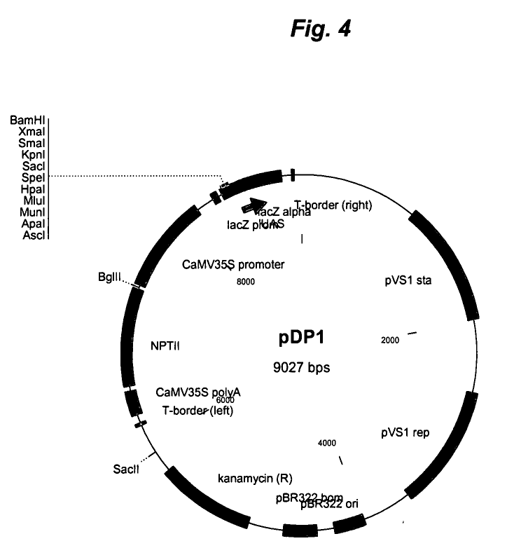

The PpENA1, SCENA1cDNAs and modified versions of same may be cut from their respective cloning vectors and introduced into the complementary sites of the pDP1 binary plasmid (FIG. 4; Rice—GAL4 UAS activation tagged lines, Hyg resistance).

The PpENA1, ScENA1 cDNAs and truncated versions of same may be cut from their respective cloning vectors and introduced into the complementary sites of the pJIT60 shuttle vector. The cDNAs or fragments thereof are cut from pJIT60 with the CaMV35S promoter region and terminator and transferred to the complementary sites of the pPG1 binary vector (FIG. 6; Barley-35S constitutive expression, Hyg resistance).

### Example 11

### Plant Transformation

PpENA1, ScENA1 and the modified versions of same, cloned into the binary vectors described above may then be introduced into the Agrobacterium strain GV3101 by electroporation, as described in Molecular Cloning: A Laboratory Manual. J. Sambrook, E. F. Fritsch, T. Maniatis 2nd ed. Cold Spring Harbor, N.Y.: Cold Spring Harbor Laboratory Press, 1989. The resultant bacteria were used to transform plants by vacuum infiltration, as described in Clough, S. J. and Bent, A. F. (1998) Plant Journal 16:735-743.

In some instances where plants are not amenable to transformation by Agrobacterium, plants may be transformed using particle bombardment, as described in Klein et al. (1988) PNAS 85(12): 4305-4309. Antibiotic or herbicide resistant transgenic plants were selected and subjected to physiological stress experiments.

The effects of a transgene on plant function can be measured at several levels, and one of the most comprehensive methods is to use whole genome microarrays. In this work, the pleiotropic effects of expression of ENA sequences on the levels of expression of all other genes in the Arabidopsis genome may be measured using whole genome microarrays on tissue from various parts of the plant.

### Example 12

### Physiological Stress Experiments

The salt tolerance of plants expressing ENA sequences may be measured using a variety of techniques known in the art. For example, visual symptoms may be documented using digital photography. Growth may be quantified by measurement of root and shoot fresh and dry weights after two to six weeks growth in short day conditions; and in this same tissue, the extent of accumulation of a wide range of elements (including Na⁺ and K⁺) may be quantified using inductively-coupled plasma spectroscopy, for example as described in Lahner et al. (2003) Nature Biotechnology 21, 1215-1221.

Unidirectional influx and efflux of Na⁺ was measured using radioactive ²²Na⁺ as a tracer for Na⁺ fluxes, as described in Essah et al. (2003) Plant Physiology 133: 307-318. These assays will be performed in Arabidopsis and also in regenerated calli of rice and barley as described in Kumria et al. (2001) Plant Cell Tissue and Organ Culture 67: 63-71, Przetakiewicz et al. (2003) Plant Cell Tissue and Organ Culture 73: 245-256 and Shankhdhar et al. (2000) Biologia Plantarum 43: 477-480

In addition, activity of the ENA1 may be assayed directly by expression of the gene product in Xenopus oocytes, for example as described in Miller & Zhou (2000) Biochim Biophys Acta. 1465(1-2):343-58 and measurement of outward currents induced by expression of ENA1.

### Example 13

### Immunolocalisation/detection of PpENA Proteins

Antibodies to the various Na⁺ pumping ATPases of the present invention may be raised by a method known in the art. The antibodies may be either monoclonal antibodies, polyclonal antibodies or recombinant antibodies.

An antigen-binding portion of an antibody may also be produced. In this regard, an antigen-binding portion of an antibody molecule includes a Fab, Fab′, F(ab′)₂, Fv, a single-chain antibody (scFv), a chimeric antibody, a diabody or any polypeptide that contains at least a portion of an immunoglobulin that is sufficient to confer specific antigen binding.

Antibodies may be generated using methods known in the art. For the production of antibodies, various hosts including goats, rabbits, rats, mice, humans, and others, may be immunized by injection with the a Na⁺ pumping ATPase or a suitable fragment thereof, including a suitable synthetic peptide of the Na⁺ pumping ATPase. Depending on the host species, various adjuvants may be used to increase immunological response. Such adjuvants include Freund's adjuvant, mineral gels such as aluminium hydroxide, and surface-active substances such as lysolecithin, pluronic polyols, polyanions, peptides, oil emulsions, keyhole limpet hemocyanin, and dinitrophenol.

A polyclonal antibody is an antibody that is produced among or in the presence of one or more other, non-identical antibodies. In general, polyclonal antibodies are produced from B-lymphocytes. Usually, polyclonal antibodies are obtained directly from an immunized subject, such as an immunized animal.

Monoclonal antibodies may be prepared using any technique that provides for the production of antibody molecules by continuous isolated cells in culture.

These include, but are not limited to, the hybridoma technique, the human B-cell hybridoma technique, and the EBV-hybridoma technique. Methods for the preparation of monoclonal antibodies are as generally described in Kohler et al. (1975) Nature 256:495-497, Kozbor et al. (1985) J. Immunol. Methods 81:31-42, Cote et al. (1983) Proc. Natl. Acad. Sci. 80:2026-2030, and Cole et al. (1984) Mol. Cell. Biol. 62:109-120.

Antibody fragments that contain specific binding sites may be generated by methods known in the art. For example, F(ab′)₂ fragments can be produced by pepsin digestion of the antibody molecule, and Fab fragments can be generated by reducing the disulfide bridges of the F(ab′)₂ fragments. Alternatively, Fab expression libraries may be constructed to allow rapid and easy identification of monoclonal Fab fragments with the desired specificity, as described in Huse, W. D. et al. (1989) Science 254:1275-1281.

Antibody molecules and antigen-binding portions thereof may also be produced recombinantly by methods known in the art, for example by expression in E. coli/T7 expression systems. A suitable method for the production of recombinant antibodies is as described in U.S. Pat. No. 4,816,567.

Various immunoassays may be used for screening to identify antibodies having the desired specificity. Numerous protocols for competitive binding or immunoradiometric assays using either polyclonal or monoclonal antibodies are known in the art.

It is specifically contemplated that two antigen sequences may be chosen and tested for their ability to produce antibodies capable of recognizing and interacting with the parent native sequence. One antigen sequence (Gly-Ser-Gly-Asp-Lys-Arg-His-Glu-Asn-Leu-Asp-Glu-Asp-Gly; SEQ ID NO. 43) represents a synthetic peptide antigen derived from PpENA1. A second sequence (Gly-Lys-Pro-Leu-Ser-Lys-Trp-Glu-Arg-Asn-Asp-Ala-Glu-Lys; SEQ ID NO. 44) represents a synthetic peptide antigen derived from PpENA2.

In both cases, suitable antibodies produced recognize the original peptide and with the parent protein but do not cross-react with the alternative sequence or protein. Each synthetic peptide includes an extra Cys residue at the carboxyl end to allow coupling of the peptide to carrier proteins.

The synthetic peptides may be coupled via their cysteine thiol groups to carrier proteins using Imject Maleimide Activated Carrier Proteins (KLH or Ovalbumin) from Pierce Chemical Company according to the manufacturer's directions.

Young Balb/c mice will be used for immunization. Three subcutaneous injections of peptide-KLH conjugates mixed with Freund's adjuvant are delivered at fifteen days intervals. The first injection uses complete adjuvant while the two following booster injections use incomplete adjuvant.

Antibody titre may be measured in an ELISA system using synthetic peptide coupled to Ovalbumin to coat the plates. Cross-reaction of the antibodies with the original complete protein may be assessed by ELISA and by Western blot.

Monoclonal antibodies-producing hybridoma cell lines may be established by fusion of mice NS-1, SP/20 or other myeloma cells with splenocytes derived from the animals immunised as above according to the technique of Kohler and Milstein (1975) Nature 256:495-497.

### Example 14

### Yeast Growth and Complementation

Saccharomyces cerevisiae (B31 strain or the B31 mutant MAT a ade2 ura3 leu2 his3 trp1 ena1Δ::HIS3::ena4Δ nha1::LEU2) was grown overnight in YPD at 37° C. with shaking. For transformation, yeast cells were washed twice with TE buffer, resuspended in 1 ml of TE buffer containing 0.1 M lithium acetate and were incubated at 30° C. for 1 hour. Yeast cells (200 ul) were used with 2 ul of salmon sperm DNA (10 mg/ml), ˜1 ug of pYES3/PpENA1 DNA, 1 ml 40% PEG 4000, 1×TE pH 7.5 and 0.1 M lithium acetate. Reagents were mixed and incubated at 30° C. for 30 min. Yeast were heat shocked at 42° C. for 15 min and washed once with 1 ml of TE before being resuspended in 200 ul of TE and plated onto SC media lacking uracil.

### Example 15

### Moss Growth Conditions

Physcomitrella patens (Hedw.), derived from a wild type collected in Gransden Wood in Huntingdonshire, UK (Ashton and Cove (1977) Mol Gen Genet. 154: 87-95) was grown at 22° C. on cellophane disks placed on solid minimal media (Ashton et al. (1979) Planta 144: 427-435) supplemented with NH₄ tartrate (0.5 g/L). Standard growth conditions were 16 h white light (fluorescent tubes, GRO-LUX, 100 μmol m⁻² sec⁻¹) and 8 h darkness.

### Example 16

### Production of Physcomitrella patens PpENA1 Mutant

To knockout PpENA1 in Physcomitrella patens using homologous recombination a construct was generated by digesting pENTR-D/PpENA1 with C/al and inserting the nptII selective cassette. The nptII cassette contains the CaMV 35S promoter, the nptII gene and the CaMV terminator. The nptII cassette was obtained by digesting pMBL6 (www.moss.leeds.ac.uk) with Clal and inserting it into the middle of the PpENA1 gene generating pCL247. To linearise pCL247 prior to transformation, the plasmid was digested with EcoRI.

Transformation was done using a PEG and heat shock based method.

For transformation Physcomitrella was subcultured on complete media containing;

| | | |
|---|---|---|
| CaNO₃•4H₂O | 0.8 | g/l |
| MgSO₄•7H₂O | 0.25 | g/l |
| FeSO₄•7H₂O | 0.0125 | g/l |
| Agar | 7 | g/l |
| CuSO₄•5H₂O | 0.055 | mg/l |
| ZnSO₄•7H₂O | 0.055 | mg/l |
| H₃BO₃ | 0.614 | mg/l |
| MnCl₂•4H₂O | 0.389 | mg/l |
| CoCl₂•6H₂O | 0.055 | mg/l |
| KI | 0.028 | mg/l |
| NaMoO₄•2H₂O | 0.025 | mg/l |
| Glucose | 5 | g/l |
| NH₄-tartrate | 0.5 | g/l |
| Kpi-buffer (pH 7) | 0.25 | g/l |

and one week old protonema was used for the production of protoplasts (essentially as described in Hohe et al., (2004) Current Genetics vol: 44 (6):339-347). Driselase was dissolved in 8% mannitol. Approximately 2g of protonema was harvested and incubated for 30 min in 20 ml of 1% driselase, 8.5% mannitol at 25° C. The tissue was filtered through 100 μm mesh, left for 15 min and filtered through 70 μm filter. The protoplasts were sedimented by a 5 min, 200g centrifugation. Protoplasts were washed in 8.5% mannitol twice and protoplast density estimated using a haemocytometer. Protoplasts were suspend at a concentration of 1−1.5×10⁶/ml in MMM buffer (8.5% mannitol, 15 mM MgCl₂, 0.1% MES, pH 5.6). 10-30 μg DNA was added to 300 μL of protoplasts, mixed gently and 300 μl PEG (7% mannitol, 0.1M Ca(NO₃)₂, 35-40% (w/v) PEG 4000, 10 mM Tris, pH 8) was added. The protoplasts were heat shocked for 5 min at 45° C. and brought back to room temperature for 5-10 min, mixing occasionally. The transformed protoplasts were kept in darkness at room temperature for 12-20 hours and then resuspend in 3 ml 8.5% mannitol and 3 ml 42° C. molten top layer medium (complete medium with 66g/1 mannitol, 1.4% agar). The protoplasts were plated on cellophane covered mannitol plates (complete medium with 66g/1 mannitol, 0.7% agar). Selection was initiated after 6-7 days by transferring the top layer to selective plates containing 25 μg/l geneticin.

To confirm integration a nested or semi-nested PCR was performed on genomic DNA purified from resistant moss according to Schlink and Reski (2002) Plant Mol Biol Rep 20: 423a-423f. The primers used to determine the site of integration were SEQ ID Nos. 46, 75, 76, 77, 78, 79 (shown in Table 4) and were annealed to the selective cassette (P35S-npt/1-CaMVter) and to the genomic sequence situated 5′ or 3′ of the PpENA1 clone. The PCR check of the 5′ end was done using oCL148-oCL76 for the first PCR and oCL149-oCL100 for the second PCR. The PCR check of the 3′ end was done using PpENA1R-oCL77 for the first PCR and oCL101-PpENA1R for the second. The expected size of the fragment if insertion occurred was 1429 bp and 1835 bp.

TABLE 4

| | | |
|---|---|---|
| Gene | Forward Primer | Reverse Primer |

| | | |
|---|---|---|
| PpENA1 | AAGGCATTACCTGGGAGTGGA | ACAGCATGGGTGCGGATTCT |
| | (SEQ ID No. 45) | (SEQ ID No. 46) |
| AtGAP | TGGTTGATCTCGTTGTGCAGGTCTC | GTCAGCCAAGTCAACAACTCTCTG |
| | (SEQ ID No. 47) | (SEQ ID No. 48) |
| AtTUB | ATGTGGGTCAGGGTATGGAA | CCGACAACCTTCTTAGTCTCCTCT |
| | (SEQ ID No. 49) | (SEQ ID No. 50) |
| AtACT | GAGTTCTTCACGCGATACCTCCA | GACCACCTTTATTAACCCCATTTACCA |
| | (SEQ ID No. 51) | (SEQ ID No. 52) |
| AtCYC | TGGCGAACGCTGGTCCTAATACA | CAAAAACTCCTCTGCCCCAATCAA |
| | (SEQ ID No. 53) | (SEQ ID No. 54) |
| PpTUB | GAGTTCACGGAAGCGGAGAG | ATATCTTTCAGGCTCCACCGA |
| | (SEQ ID No. 55) | (SEQ ID No. 56) |
| PpEFA | GCCAAGAAGAAGGTAATAGTGCG | ACGTCTGCCTCGCTCTAGC |
| | (SEQ ID No. 57) | (SEQ ID No. 58) |
| PpACT | GCGAAGAGCGAGTATGACGAG | AGCCACGAATCTAACTTGTGATG |
| | (SEQ ID No. 59) | (SEQ ID No. 60) |
| PpHIS | CGTCCAGGAACAGTCGCTCTT | TTCACAGCCTACGCCCTCTCT |
| | (SEQ ID No. 61) | (SEQ ID No. 62) |
| PpRNA | CAACGACGATACTTCTTGGCTG | GCTGCTCCACCAGTCCTGCTA |
| | (SEQ ID No. 63) | (SEQ ID No. 64) |
| Os GAP | GGGCTGCTAGCTTCAACATC | TTGATTGCAGCCTTGATCTG |
| | (SEQ ID No. 65) | (SEQ ID No. 66) |
| HvHSP | CGACCAGGGCAACCGCACCAC | ACGGTGTTGATGGGGTTCATG |
| | (SEQ ID No. 67) | (SEQ ID No. 68) |
| HvGAP | GTGAGGCTGGTGCTGATTACG | TGGTGCAGCTAGCATTTGAGAC |
| | (SEQ ID No. 69) | (SEQ ID No. 70) |
| HvCYC | CCTGTCGTGTCGTCGGTCTAAA | ACGCAGATCCAGCAGCCTAAAG |
| | (SEQ ID No. 71) | (SEQ ID No. 72) |
| HvTUB | AGTGTCCTGTCCACCCACTC | AGCATGAAGTGGATCCTTGG |
| | (SEQ ID No. 73) | (SEQ ID No. 74) |
| oCL76 | GACCACTGTCGGCAGAGGCATC | |
| | (SEQ ID No. 75) | |
| oCL77 | GACCACTGTCGGCAGAGGCATC | |
| | (SEQ ID No. 76) | |
| oCL100 | GGCAATGGAATCCGAGGAGGT | |
| | (SEQ ID No. 77) | |
| oCL101 | GGTATCAGAGCCATGAATAGGTC | |
| | (SEQ ID No. 78) | |
| oCL148 | CGACGACGATCCTGTGCTC | |
| | (SEQ ID No. 79) | |
| oCL149 | CCGTTGACAGTTGAGTAACACC | |
| | (SEQ ID No. 80) | |
| ANTIF1 | AAAGGATCCGGGGACTTGTTGACGTTCGAG | |
| | (SEQ ID No. 81) | |
| ANTIR1 | AAAAAGCTTTTGATGGCCTTGGAAATCTT | |
| | (SEQ ID No. 82) | |

### Example 17

### Plant Growth Conditions

Arabidopsis thaliana was transformed via the floral dip method (Clough et al., (1998). Plant Journal 16, 735-743) using Agrobacterium tumefaciens strain GV3101::pMP90(R^{K}) with the binary vectors pAJ53, pAJ65 and pAJ66. Plants (T₃) used in salt sensitivity assays were grown in an artificial soil medium (3.6 L perlite-medium grade, 3.6 L coira and 0.25 L river sand) or on agar plates containing ½ MS media (Murashige, T. and Skoog, F. (1962). Physiol. Plant. 15: 473-497.) in a growth room at 22° C. with 8 hours light and 16 hours darkness. Seedlings (10 days old) transformed with the binary constructs were selected by spraying every second day for one week with 100 mg.L⁻¹ BASTA (AgrEvo, Düsseldorf, Germany). Plants in artificial soil were watered with a hydroponic nutrient mix (Table 5) once per week.

TABLE 5

Final concentration

Nutrients

| | | |
|---|---|---|
| Ca(NO₃)₂•4H₂O | 2 | mM |
| KNO₃ | 15 | mM |
| MgSO₄•7H₂O | 0.5 | mM |
| NaH₂PO₄•H₂O | 500 | μM |
| NH₄NO₃ | 15 | mM |
| NaFeEDTA | 25 | μM |
| H₃BO₃ | 200 | μM |
| Micronutrients | 0.05 | μM |

Micronutrients

| | | |
|---|---|---|
| Na₂MoO₄•2H₂O | 1 | μM |
| NiCl₂•6H₂O | 1 | μM |
| ZnSO₄•7H₂O | 2 | μM |
| MnCl₂•4H₂O | 4 | μM |
| CuSO₄•5H₂O | 2 | μM |
| CoCl₂•6H₂O | 1 | μM |

Embryogenic nodular units of Oryza sativa L. cv Nipponbare arising from scutellum-derived callus were inoculated with supervirulent A. tumefaciens strains EHA105 and AGL1 (carrying the pC-4956:ET15 plasmid). Plants were transformed using the binary vectors pAJ54 and pAJ55. Hygromycin-resistant shoots (50 mg.L-1) were regenerated after nine weeks according to the protocol described by Sallaud et al. (2004). Plant Journal. 39(3): 450-464. After selection and regeneration in tissue culture plants were transferred to soil and placed in a glasshouse.

Hordeum vulgare L. cv Golden Promise callus derived from immature embryos was transformed using an Agrobacterium tumefaciens-mediated transformation protocol developed by Tingay et al. (1997) Plant Journal. 11(6): 1369-1376 and modified by Matthews et al. (2001) Molecular Breeding 7(3): 195-202. Plants were transformed using the binary vectors pAJ54 and pAJ55. After regeneration and selection in tissue culture plants were transferred to soil and placed in a glasshouse.

### Example 18

### Salt Sensitivity Assays

Moss stress experiments were carried out on 5 week old gametophytes. Abiotic stress was induced by transferring the cellophane with the Physcomitrella to media or filter disks containing 60 or 100 mM NaCl. Extra CaCl₂ was added to the media containing NaCl to keep the level of available Ca²⁺ constant. The amount of CaCl₂ added was determined using MinTeq ver2.30 (http://www.lwr.kth.se/English/OurSoftware/vminteq/). Arabidopsis seeds were surface sterilised in 50% Domestos for 5 minutes and were rinsed several times in sterile water before being plated onto ½ MS media with 0.6% phytagel supplemented with 100 mM, 150 mM, 200 mM, 250 mM or 300 mM NaCl. Seed was vernalised in the dark overnight at 4° C. and plates were placed in a growth room under the conditions described above.

### Example 19

### DNA and RNA Extractions

Genomic DNA was extracted from young leaves of Arabidopsis using a hot CTAB method (Lassner et al., 1989) Molecular & General Genetics 218, 25-32). Genomic DNA was extracted from leaves of barley and rice using a protocol from Pallotta et al., (2000). Theoretical and Applied Genetics 101: 1100-1108.

Total RNA was extracted from young leaves of Arabidopsis, barley and rice using Trizol reagent (Invitrogen Corporation, Carlsbad, Calif., USA) according to the manufacturer's instructions.

### Example 20

Analysis of Transgene Copy Number

Genomic DNA (10 μg) was digested for 6-18 h at 37° C. with 100 U BamH1. Digested DNA was separated on 1% (w/v) agarose gels and DNA fragments were transferred to a nylon membrane using the Southern method. The nylon membrane was neutralised in a solution of 2×SSC. Membranes were blotted dry and dried under vacuum at 80° C. prior to probing. Prehybridisation of the membranes was conducted in a 6×SSC, 1×Denhardt's III solution (2% w/v BSA, 2% w/v Ficoll 400 and 2% PVP), 1% (w/v) SDS and 2.5 mg denatured salmon sperm DNA for a minimum of 4 h at 65° C. Hybridisation mixture (10 ml) containing 3×SSC, 1×Denhardt's III solution, 1% (w/v) SDS and 2.5 mg denatured salmon sperm DNA was used to replace the discarded prehybridisation mixture. DNA probes were radiolabelled with [α-³²P]-dCTP, using a Megaprime DNA labelling kit according to the manufacturer's directions (Amersham, UK). The probe was hybridised for 16 h at 65° C. The membranes were washed sequentially for 20 min at 65° C. in 2×SSC containing 0.1% (w/v) SDS, with 1×SSC/0.1% (w/v) SDS and with 0.5×SSC/0.1% (w/v) SDS. Membranes were blotted dry, sealed in plastic and RX X-ray film was exposed to the membrane at −80° C. for 24-48 h, using an intensifying screen.

### Example 21

### Flame Photometry

Tissue for flame photometry was rinsed briefly in deionised water, dried, weighed then digested overnight in 1-2 ml of 1% nitric acid at 85° C. After cooling and diluting as necessary, samples were loaded into a Sherwood model 420 flame photometer and the Na⁺ and K⁺ concentrations were recorded.

### Example 22

### Cloning and Construction of the Recombinant Expression Vector

The QIAexpress (Qiagen) recombinant protein expression system was employed to express and purify a region of the PpENA protein (amino acids P150 to K244). The P150/K244 peptide sequence was amplified from the PpENA1 cDNA with the antiF1/R1 primer set (Table 4). The primers were designed to add a 5′ BamHI sequence and 3′ HindIII sequence to the PCR amplicon. The P150/K244 PCR product was cut with BamHI and HindIII, purified using Qiagen PCR purification columns, following the manufacturer's instructions, and cloned into a BamHI-HindIII double digested pQE-30 expression vector.

### Example 23

### Expression and Purification of the Recombinant PpENA Peptide

Recombinant plasmids were transformed into competent M15 E. coli cells (Qiagen, USA) by heat shock treatment. Cells were plated on LB plates containing 25 μg/ml kanamycin and 100 μg/ml ampicillin and incubated overnight at 37° C. Individual colonies were selected and inoculated into 5 ml of LB containing both antibiotics and grown at 37° C. with constant shaking for approximately 12 hrs. 500 μl of the starter culture was removed and inoculated into 10 ml of 37° C. LB (containing 25 μg/ml kanamycin and 100 μg/ml ampicillin) and the culture was grown at 37° C. with shaking until the OD₆₀₀ reached 0.8. Protein expression was induced by the addition of IPTG to a final concentration of 2 mM. Three hours after induction, cells were harvested by centrifugation and the pellet resuspended in 1 ml of lysis solution (50 mM NaH₂PO₄, 300 mM NaCl, 1% Triton, 5 mM imidazol, pH 8.0) containing 1 mg, 0.3 mg and 0.3 mg of Lysozyme, RNase and DNase, respectively. The solution was then left on ice for 30 min. Cells were lysed by a combination of rapid freeze-thawing (in liquid nitrogen) followed by sonication (6×6 s) at 40 W in a Branson B-12 Sonifier and the cellular debris removed by centrifugation at 10,000 rμm for 10 min. A 50% slurry of Ni/nitriloacetic acid resin (Qiagen, USA) in lysis buffer was added to the supernatant and the recombinant proteins separated from endogenous proteins by virtue of their histidine tag. Contaminating proteins were removed by a series of three individual washing steps: Step 1, four washes with 50 mM NaH₂PO₄, 300 mM NaCl, 5 mM immidazol, pH 8.0; Step 2, three washes with 50 mM NaH₂PO₄, 300 mM NaCl, pH 6.0; Step 3, three washes with 100 mM KH₂PO₄, pH 6.0. The purified protein was eluted from the resin by the addition of 100 mM KH₂PO₄, 2 mM EDTA, pH 3.0. The eluate containing the recombinant protein was then titrated to pH 7.0 by the addition of 100 mM KH₂PO₄, 2 mM EDTA, pH 10.0. Protein concentration was determined spectrophotomerically (Shimadzu UV-160 A) at 280 nm and by comparison with a BSA standard curve. The purified protein was visualised on a 12.5% polyacrylamide gel with protein markers in the 7 to 200 kDa range (Prestained Broad-Range, BIORAD USA).

### Example 25

### PpENA1 Antibody Production

Balb/c mice were immunised with 50 mg of recombinant peptide preparation coupled to keyhole limpet hemocyanin carrier protein mixed with Freund's adjuvant. Four sub-cutaneous injections were given at 3 week intervals. Three days after the last injection, spleen cells were fused with NS1 mouse myeloma cells. To detect anti-PpENA antibodies by ELISA, 50 ml of each hybridoma supernatant was used in 96-well plates coated with the same peptide used for immunisation but coupled to ovalbumin carrier protein. The peptide conjugate was coated onto the plates in 0.1M carbonate buffer, pH 9.6 at 4° C. overnight. The plates were washed and blocked with 200 ml of boiled casein for 60 min and washed again. After incubation with the hybridoma supernatant at 37° C. for 1.5 h, the plates were washed again and incubated with a rabbit anti-mouse IgG-HRP conjugated antibody at a dilution of 1:10,000. The plates were incubated at 60 min at 37° C., washed and developed with TMB. Selected hybridoma were subcloned by limiting dilution. Specificity of positive hybridoma were further screened by western blotting.

### Example 26

### Quantitative PCR Analysis of PpENA1 mRNA

Total RNA (2 μg) was used in cDNA reactions using a Superscript III cDNA synthesis kit (Invitrogen). The primer pairs for control genes were designed for each plant variety and the moss PpENA1 gene and are listed in Table 4. Stock solutions of the PCR product were prepared from cDNAs and were purified and quantified by HPLC.

The leaf-derived cDNA (1 μl) was amplified in a reaction containing 10 μl QuantiTect SYBR Green PCR reagent (Qiagen, Valencia, Calif., USA), 3 μl each of the forward and reverse primers at 4 μM, and 3 μl water. The amplification was effected in a RG 2000 Rotor-Gene Real Time Thermal Cycler (Corbett Research, Sydney, Australia) as follows; 15 min at 95° C. followed by 45 cycles of 20 s at 95° C., 30 s at 55° C., 30 s at 72° C. and 15 s at 80° C. A melt curve was obtained from the PCR product at the end of the amplification by heating from 70° C. to 99° C. During the amplification, fluorescence data was acquired at 72° C. and 80° C. in order to gauge the abundance of the individual genes in the cDNA preparation. From the melt curve, the optimal temperature for data acquisition was determined.

Between four and six independent 20 μl PCR reaction mixes were combined and purified by HPLC (Wong et al., (2000). BioTechniques 28: 776-783) on an Agilent Eclipse DS DNA 2.1 mm×15 cm 3.5 micron reverse phase column (Agilent Technologies, Palo Alto, Calif., USA). Chromatography was performed using buffer A (100 mM triethylammonium acetate, 0.1 mM EDTA) and buffer B (100 mM triethylammonium acetate, 0.1 mM EDTA, 25% acetonitrile). The gradient was applied at a flow rate of 0.2 ml/min at 40° C., as follows: 0-30 min with 35% buffer B, 30-31 min with 70% buffer B, 31-40 min with 35% buffer B, and after 40 min, 35% buffer B. The purified PCR products were quantified by comparison of the peak area with the areas of three of the peaks in a pUC19/Hpall digest (Geneworks, Adelaide, Australia). In 2 μl of a 500 ng/μl digest, the peaks used for reference were 147 bp, representing 55 ng, 190 bp (71 ng) and 242 bp (90 ng). From these data, an average value for nanograms per unit area of a peak was calculated. This value was used to determine the mass of the purified PCR product. The value was determined with every batch of PCR products purified. The product was dried and dissolved in water to produce a 20 ng/μl stock solution. The size in base pairs and identity of PCR products was confirmed by sequencing. An aliquot of this solution was diluted to produce a stock solution containing 109 copies of the PCR product per microlitre. A dilution series covering seven orders of magnitude was prepared from the 10⁹ copies/μl stock solution to produce solutions covering 10⁷ copies/μl to 10¹ copies/μl.

Three replicates of each of the seven standard concentrations were included with every Q-PCR experiment, together with a minimum of two ‘no template’ controls. For all genes a 1:20 dilution of the cDNA was sufficient to produce expression data with an acceptable standard deviation. Four replicate PCRs for each of the cDNAs were included in each experiment.

For the Q-PCR experiments, 1 μl cDNA solution was used in a reaction containing 10 μl of QuantiTect SYBR Green PCR reagent, 3 μl each of the forward and reverse primers at 4 μM, 0.6 μl 10×SYBR Green in water (freshly diluted 10,000× in dimethyl sulphoxide) and 2.4 μl water. Reactions were performed as follows; 15 min at 95° C. followed by 45 cycles of 20 s at 95° C., 30 at 55° C., 30 s at 72° C. and 15 s at the optimal acquisition temperature (Table 11). A melt curve was obtained from the product at the end of the amplification by heating from 70-99° C. PCR products were separated by electrophoresis in 2.5% agarose-TBE-ethidium bromide gels. The Rotor-Gene V4.6 software (Corbett Research, Sydney, Australia) was used to determine the optimal cycle threshold (CT) from the dilution series, and the mean expression level and standard deviations for each set of four replicates for each cDNA were calculated.

### Example 27

### Western analysis of PpENA1 in Arabidopsis and Moss

Protein was extracted from Arabidopsis and moss samples by grinding the leaf or chloronema tissue in 200 ul of 50 mM HEPES buffer, pH 4 containing 1 mM PMSF, 1 mM benzamidine, 50 mM sodium fluoride and 1 mM protease inhibitor. Samples were centrifuged at 13,500 rμm for 5 min and the pellets were resuspended in 110 ul of 50 mM HEPES buffer, pH 4 containing 1 mM PMSF, 1 mM benzamidine, 50 mM sodium fluoride and 1 mM protease inhibitor and 30 ul of 0.225 M Tris-HCl buffer, pH8 containing 50% glycerol, 5% SDS, 0.05% bromophenol blue and 0.25 M DTT and boiled for 15 min. Samples were centrifuged at 13,500 rμm for 5 min and the solubilised membrane fraction was loaded into a 10% polyacrylamide gel.

Proteins were electrophoretically transferred from the gel to Hybond P(PVDF) membrane (Amersham, Buckinghamshire, UK). 1 ul of ovalbumin conjugated recombinant protein was spotted onto the corner of the membrane and the membrane was blocked by incubation in a 5% skim milk solution at room temperature overnight. Membranes were washed for 5 min 3 times in PBS containing 0.05% Tween20 with shaking. Protein G purified polyclonal rabbit sera diluted 1:500 in PBS containing 0.05% Tween20 was incubated with the membranes overnight. Membranes were washed for 5 min 3 times in PBS containing 0.05% Tween20 with shaking and the secondary antibody Anti-rabbit IgG-Biotin conjugate (Molecular probes, CA, USA) diluted 1:1000 in PBS containing 0.05% Tween20 was added and left to bind for 1 hour. Membranes were washed for 5 min 3 times in PBS containing 0.05% Tween20 with shaking. Streptavidin-Alkaline phosphatase (Sigma, Mo., USA) diluted 1:2000 in PBS containing 0.05% Tween20 was added and the membranes were incubated for 1 hour at room temperature. Membranes were washed for 5 min 3 times in PBS containing 0.05% Tween20 with shaking and developed in NBT/BCIP purple (Sigma, Mo., USA).

### Example 27

### PpENA1 Expression Rescues a Salt Sensitive Yeast Strain

B31 Salt-sensitive yeast were transformed with PpENA1 under control of the Gal promoter inoculated onto 300 mM NaCl plates. B31 (MAT a ade2 ura3 leu2 his3 trp1 ena1Δ::HIS3::ena4Δ nha1Δ::LEU2 transformed with pYES-ENA) was grown in SC-ura overnight. 5 serial 1 in 2 dilutions were made and 1 μl of each spotted on to either SC-ura+0.3M NaCl+glucose or SC-ura+0.3M NaCl+galactose.

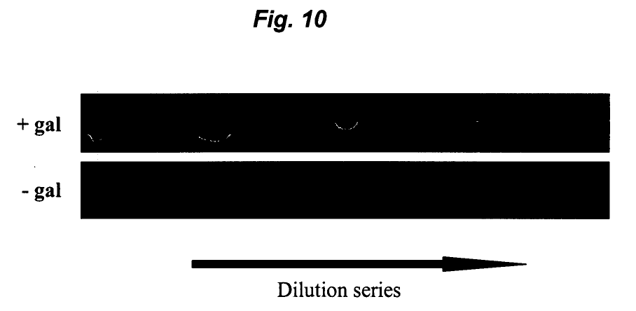

The results are shown in FIG. 10. As can be seen, Gal induced transcription of PpENA1 rescues the B31 mutant's salt sensitivity phenotype.

### Example 28

### Moss mutants defective in PpENA1 expression accumulate more Na⁺ and grow at a reduced rate

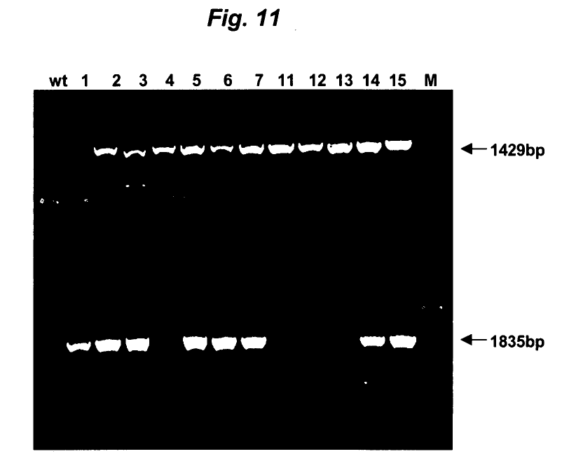

FIG. 11 shows the results of confirmation by PCR of PpENA1 disruption in genomic DNA from kanamycin resistant Physcomitrella patens transformants.

The PCR check of the 5′ end was done using oCL148-oCL76 for the first PCR and oCL149-oCL100 for the second PCR. The PCR check of the 3′ end was done using PpENA1R-oCL77 for the first PCR and oCL101-PpENA1R for the second. The expected size of the fragment if insertion occurred was 1429 bp and 1835 bp. On this basis lines 2, 3, 5, 6, 7, 14 and 15 are PpENA1 mutants.

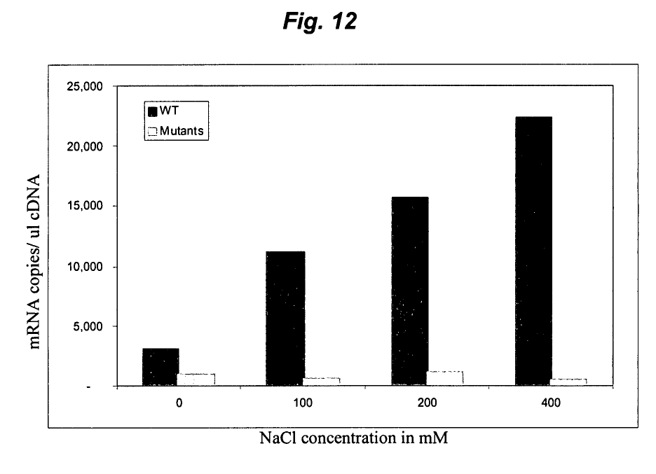

PpENA1 mRNA levels were determined by qPCR for three moss PpENA1 knockout mutants and wildtype. The results are shown in FIG. 12. PpENA1 mRNA levels increase in the wildtype as the NaCl concentration increases. The mutants are unable to synthesise PpENA1 mRNA.

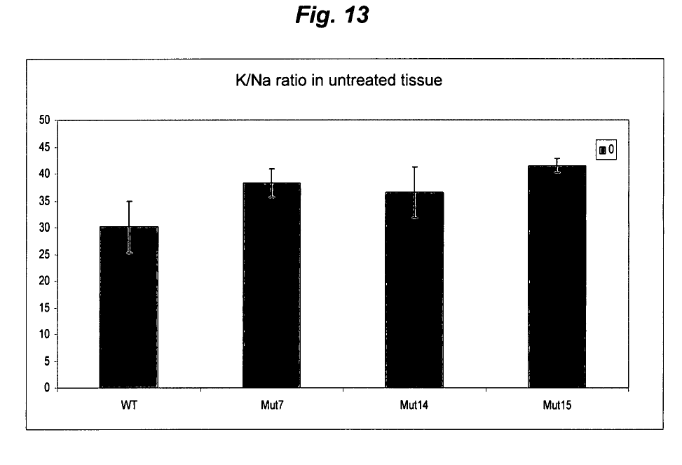

To determine the sodium and potassium concentrations in wildtype moss and the mutant moss, flame photometry was used. The results are shown in FIG. 13. As can be seen, wildtype moss is able to maintain a higher K⁺/Na⁺ ratio than the mutants at 100 mM NaCl.

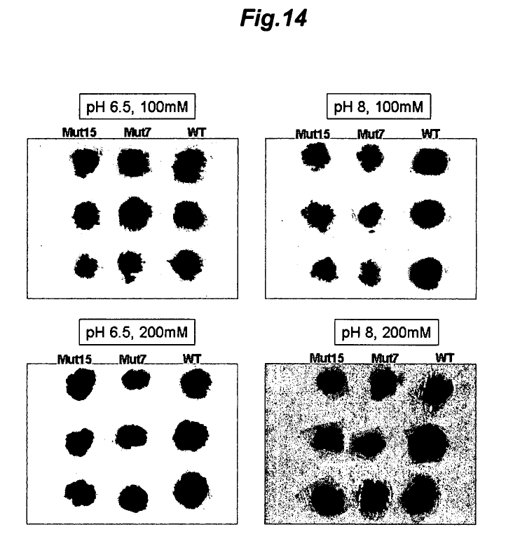

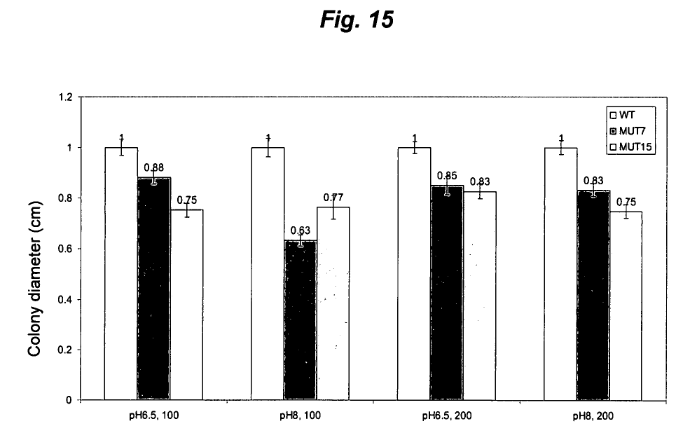

FIG. 14 shows that PpENA1 knockout mutants have reduced biomass in comparison to wildtype after 1 week on media containing 100 or 200 mM NaCl. The results are also presented graphically in FIG. 15. As can be seen, wildtype attains a larger diameter than the PpENA1 knockout mutants.

### Example 29

### Arabidopsis Transgenics Express PpENA1 at different levels

Transgenic plants were produced as described in Example 17.

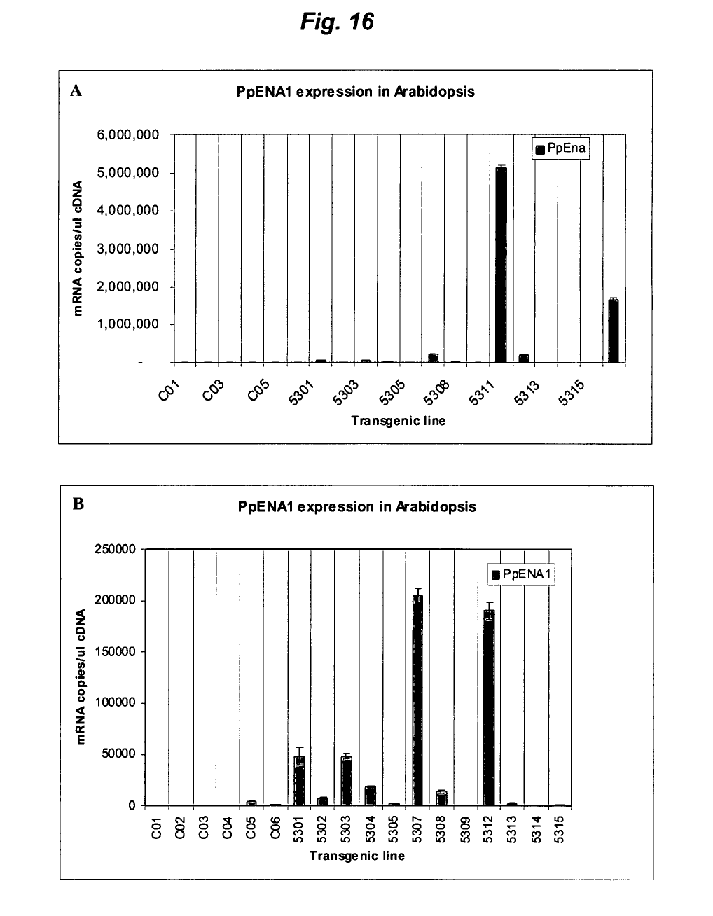

FIG. 16A and FIG. 16B show PpENA1 mRNA levels in Arabidopsis T1 transgenics constitutively expressing PpENA1. High expressing lines 5311 and 5316 have been removed from the graph in panel B. The results demonstrate that varying levels of transcription of the PpENA1 mRNA were achieved.

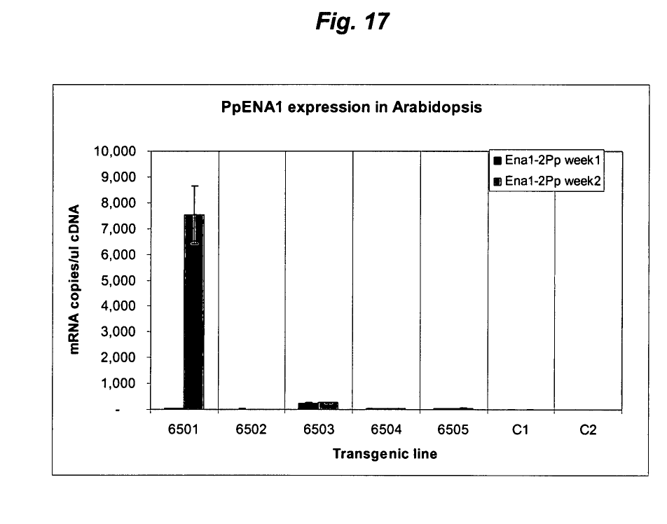

FIG. 17 shows PpENA1 mRNA levels in Arabidopsis T1 transgenics induced following exposure to 30 mM NaCl. The endogenous moss PpENA1 promoter drives expression of PpENA1 in a salt sensitive manner in line 6501.

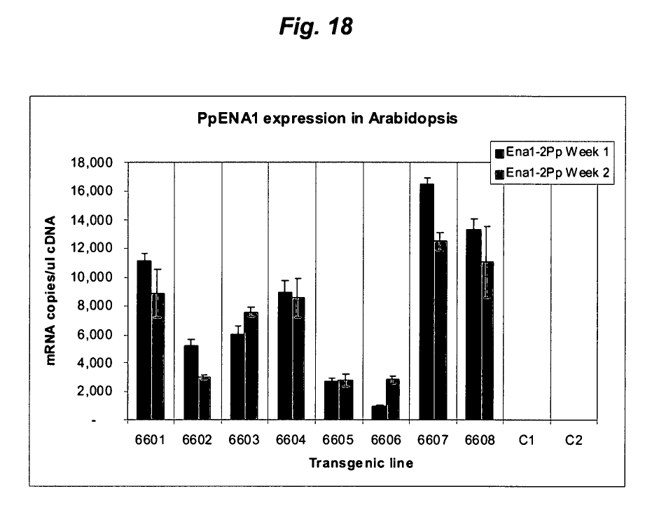

FIG. 18 shows PpENA1 mRNA levels in Arabidopsis T1 transgenics induced following exposure to 30 mM NaCl. The Arabidopsis VHAc3 promoter produces a low level of expression of PpENA1 mRNA at 30 mM NaCl.

### Example 30

### Arabidopsis Transgenics Expressing PpENA1 have altered levels of Na⁺

Table 6 shows sodium and potassium concentrations in leaf tissue of Arabidopsis T1 transgenics constitutively expressing PpENA1. As can be seen, in general transgenic plants accumulate less sodium on average than wild type or non-transgenics.

TABLE 6

| | | Na+ | K+ | |
|---|---|---|---|---|
| pAJ53 | Line | μMol/g DW | μMol/g DW | |
| 30 mM NaCl | C4 | 70.49 | 1491.80 | Wildtype and |
| 2 weeks | C5 | 139.29 | 1441.07 | non-transgenics |
| | C6 | 161.02 | 1452.54 | |
| | 14 | 124.21 | 1461.05 | |
| | 15 | 111.32 | 1356.60 | |
| | av | 121.27 | 1440.61 | |
| | std dev | 33.88 | 50.59 | |
| 30 mM NaCl | 11 | 97.20 | 1398.13 | Transgenics |
| 2 weeks | 12 | 96.20 | 1405.06 | |
| | 13 | 93.58 | 1387.16 | |
| | 16 | 121.74 | 1430.43 | |
| | av | 102.18 | 1405.20 | |
| | std dev | 13.13 | 18.37 | |

Table 7 shows sodium and potassium concentrations in leaf tissue of Arabidopsis T1 transgenics with PpENA1 transcription under control of the Arabidopsis VHAc3 promoter. Transgenic plants accumulate various levels of Na⁺ and K⁺.

TABLE 7

| | | |
|---|---|---|
| 10 mM NaCl | Na+ μMol/g DW | K+ μMol/g DW |

| Line | 3 Days | 14 Days | 3 Days | 14 Days |
|---|---|---|---|---|
| pAJ6601** | 112.43 | 38.46 | 775.15 | 463.94 |
| pAJ6602* | 44.33 | 43.27 | 642.04 | 586.54 |
| pAJ6603** | 35.26 | 296.58 | 466.85 | 802.28 |
| pAJ6604** | 34.44 | 124.66 | 476.82 | 705.09 |
| pAJ6605* | 29.30 | 257.05 | 355.41 | 738.24 |
| pAJ6606* | 56.39 | 133.90 | 340.85 | 657.42 |
| pAJ6607** | 88.08 | 350.35 | 467.48 | 678.01 |
| pAJ6608** | 228.26 | 55.69 | 489.13 | 273.61 |
| Control | 105.34 | 94.50 | 479.58 | 793.54 |
| Av (n = 4) | | | | |
| Control | 27.98 | 31.27 | 167.04 | 523.95 |
| Std dev | | | | |

### Example 31

### Arabidopsis Transgenics Expressing PpENA1 have a growth advantage on 100 mM NaCl

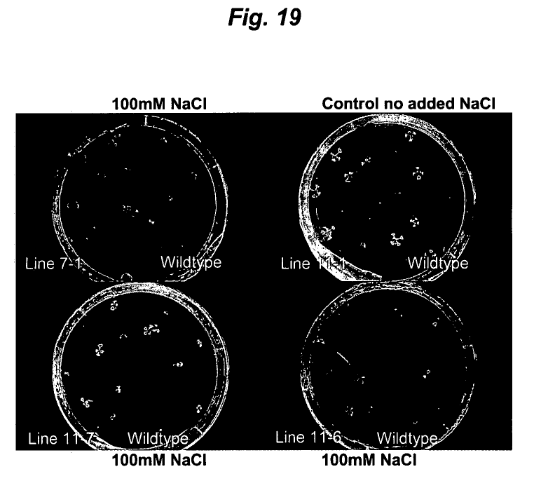

FIG. 19 shows that T3 Transgenic Arabidopsis plants constitutively expressing PpENA1 may have a growth advantage on 100 mM NaCl when compared to wildtype.

### Example 32

### Rice Transgenics Contain PpENA1

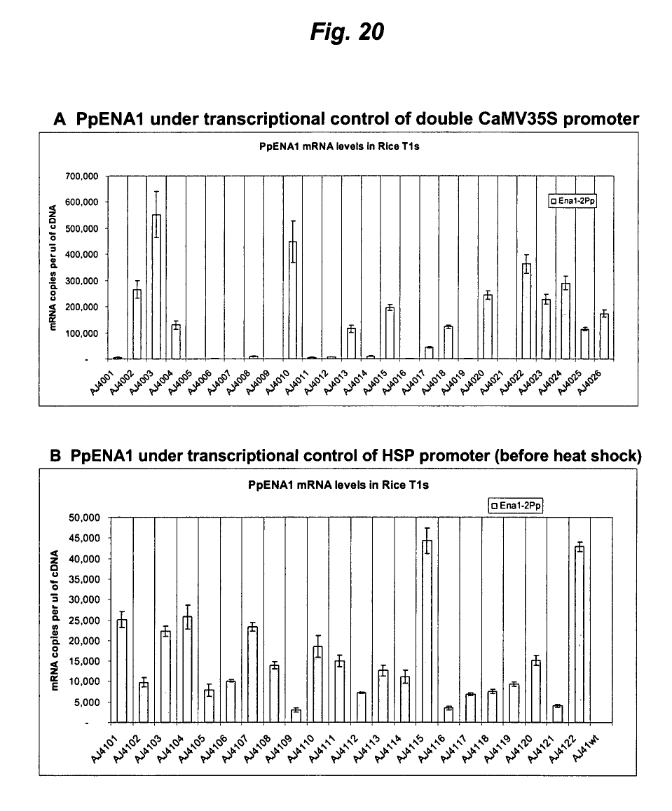

FIG. 20 shows the results of q-PCR expression of PpENA1 in rice transgenics containing the pAJ55 binary construct. Southern analysis (not shown) using a PpENA1 probe demonstrated that a number of the lines possess more than one copy of the PpENA1 gene.

### Example 33

### Putative Barley Transgenics Containing PpENA1

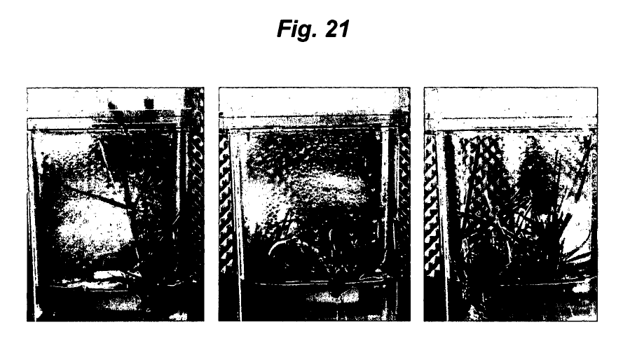

FIG. 21 shows hygromycin resistant barley plants transformed with pAJ54 and pAJ55 in tissue culture.

### Example 34

### Detection of PpENA1 Protein in Moss and Transgenic Arabidopsis

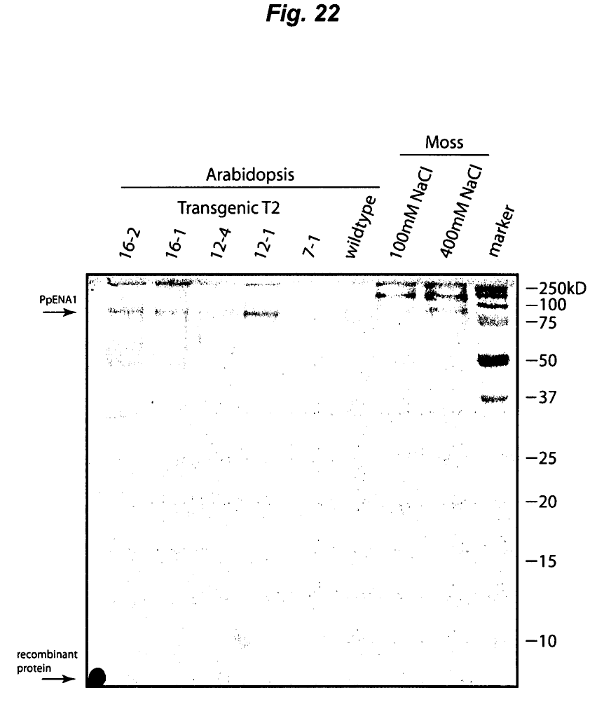

FIG. 22 shows Western analysis of Arabidopsis T2 transgenics and salt treated moss probed with PpENA1 antibody. The ˜100 kDa band indicated by the arrow may represent the position of the PpENA1 protein in the protein extracts from moss and Arabidopsis.

### Example 35

### T1 rice transgenics constitutively expressing PpENA1 have altered levels of Na⁺ in their shoots

Transgenic rice plants were generated as described previously herein. T1 Rice plants were germinated on Petri dishes on wet filter paper for 1 week before being transferred to a supported hydroponic growth setup containing the media shown in Table 8.

TABLE 8

| | | | | | |
|---|---|---|---|---|---|
| | | | | Vol. of | |
| | | Mass (g) | Stock | stock (ml) | |
| | | for 1 litre | solution | for 1 litre | Final |
| | Formula | of stock | conc. | culture | conc. |
| Salts | Weight | solution | (nM) | solution | (mM) |

Macronutrients

| 1 | NH₄NO₃ | 80.0 | 80.0 | 1.0 | 5.0 | 5.0 |
|---|---|---|---|---|---|---|
| | KNO₃ | 101.1 | 101.1 | 1.0 | | 5.0 |
| 2 | Ca(NO₃)₂•4H₂O | 236.1 | 94.4 | 0.4 | 5.0 | 2.0 |
| 3 | MgSO₄•7H₂O | 246.5 | 98.6 | 0.4 | 5.0 | 2.0 |
| | KH₂PO₄ | 136.1 | 2.72 | 0.02 | | 0.1 |
| 4 | Na₂SiO₃ | 122.0 | 112 mls | 0.5 | 1.0 | 0.5 |
| | | | of 4.45 M | | | |
| | | | liquid | | | |
| | | | stock | | | |
| 5 | NaFe(III)EDTA | 367.1 | 18.4 | 0.05 | 1.0 | 0.05 |
| | | | | (mM) | | (μM) |

Micronutrients

| | | | | | | |
|---|---|---|---|---|---|---|
| 6 | H₃BO₃ | 61.8 | 3.09 | 50.0 | 1.0 | 50.0 |
| | MnCl₂•4H₂O | 197.9 | 0.990 | 5.0 | | 5.0 |
| | ZnSO₄•7H₂O | 287.5 | 2.875 | 10.0 | | 10.0 |
| | CuSO₄•5H₂O | 249.7 | 0.125 | 0.5 | | 0.5 |
| | Na₂MoO₃ | 242.0 | 0.024 | 0.1 | | 0.1 |

At the appearance of the 3^{rd} leaf 50 mM NaCl was added to the growth medium. The 4^{th} leaf was harvested 11 days after emergence and the sodium content was determined as described in Example 21. The data is shown in Table 9

TABLE 9

| | mRNA | | Av. | |
|---|---|---|---|---|
| Rice | PpENA1 | [Na] | [Na] | |
| T1 Line | copies/ul | mg/kg | mg/kg | |
| 4th leaf | cDNA | dry wt | dry wt | |
| 4Os2-1 | 235380 | 460 | | |
| 4Os2-2 | 835950 | 840 | | |
| 4Os2-4 | 84275 | 2700 | | |
| 4OS2-7 | 559570 | 1170 | | |
| 4Os2-10 | 416410 | 1080 | | |
| 4Os2-12 | 1493340 | 490 | 1123.333 | |
| 4Os3-1 | 864975 | 520 | | |
| 4Os3-2 | 98370 | 580 | | |
| 4Os3-3 | 2105870 | 1060 | | |
| 4Os3-4 | 202665 | 600 | | |
| 4Os3-5 | 5040 | 620 | | |
| 4Os3-7 | 326950 | 790 | | |
| 4Os3-8 | 3240 | 1580 | | |
| 4Os3-9 | 183280 | 640 | | |
| 4OS3-10 | 625815 | 350 | | |
| 4Os3-11 | 886595 | 740 | | |
| 4Os3-12 | 240400 | 680 | 741.8182 | |
| 4Os15-1 | 788135 | 560 | | |
| 4Os15-3 | 999200 | 470 | | |
| 4Os15-4 | 958970 | 540 | | |
| 4Os15-5 | 956040 | 610 | | |
| 4OS15-6 | 50* | 310 | | |
| 4OS15-9 | 1830930 | 300 | | |
| 4Os15-10 | 104160 | 280 | | |
| 4OS15-12 | 2484580 | 470 | 442.5 | |
| 4Os17-4 | 950 | 860 | | |
| 4Os17-5 | 660 | 580 | | |
| 4Os17-6 | 15820 | 950 | | |
| 4Os17-8 | 7995 | 610 | | |
| 4Os17-10 | 55* | 680 | | |
| 4Os17-12 | 435 | 810 | 748.3333 | |
| 4Os23-1 | 1309565 | 570 | | |
| 4Os23-7 | 465860 | 530 | | |
| 4Os23-8 | 776650 | 270 | | |
| 4Os23-11 | 1427765 | 560 | | |
| 4Os23-12 | 26385 | 700 | 526 | |
| | 4th leaf average | | 767 | n = 16 |
| | for all nulls | | | |

*questionable

The above data demonstrates that the 4Os15 and 4Os23 lines show reduced Na⁺ accumulation in the shoots.

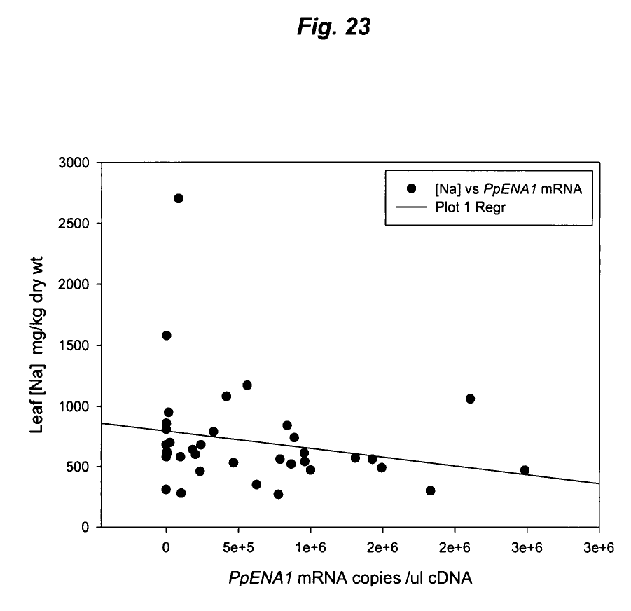

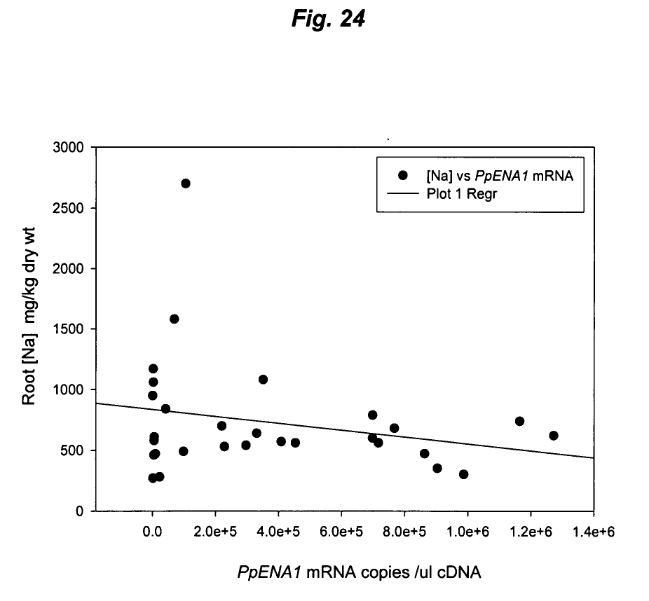

FIGS. 23 and 24 show that higher PpENA1 expression is correlated with lower shoot and root Na⁺ in T1 rice.

T2 transgenic rice plants with single transgene inserts were produced as described in Example 17 and qPCR was used to determine transgene expression levels in the root and shoot as described in Example 26. This data (not shown) demonstrates that PpENA1 is constitutively expressed at various levels in roots and shoots of T2 rice lines.

### Example 36

### PpENA1 is Expressed in Shoots of T1 Barley Lines

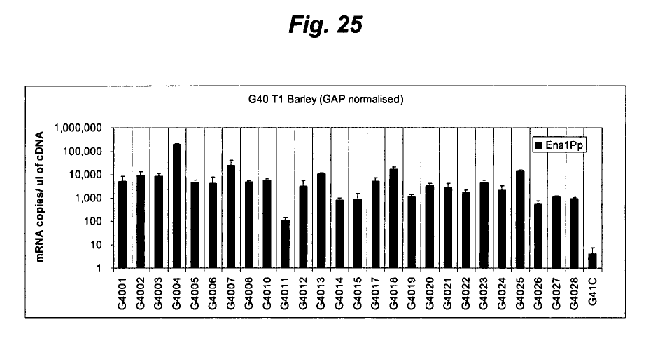

T1 barley lines were produced as described in Example 17. The level of PpENA1 expression was determined in the shoots of T1 barley lines, as shown in FIG. 25. This data indicates that PpENA1 is expressed to differing levels in the shoots of each of the lines.

### Example 37

### Cell-specific expression of PpENA1 in epidermal rice roots leads to lowered levels of Na⁺ in primary transgenic plants

PpENA1 was expressed in either the epidermal or in the xylem parenchyma cells of the rice root. Two GAL4-GFP enhancer trap rice lines were selected and transformed as described in Example 17 to express PpENA1 cell type-specifically. Transgenic lines were regenerated and subjected to a 13 d, 5 mM Na⁺ stress in hydroponic culture. The youngest fully emerged blade (YEB) was harvested and Na⁺ and K⁺ contents were measured using flame photometry. The lines expressing PpENA1 in the xylem parenchyma cells of the root accumulated more Na⁺ and similar K⁺ in the YEB than lines expressing RFP in the root xylem parenchyma cells (control). The lines expressing PpENA1 in the epidermal cells of the root accumulated less Na⁺ and similar K⁺ in the YEB than lines expressing RFP in the root epidermal cells (control).

### Example 38

### Cell-specific expression of PpENA1 in rice roots leads to altered levels of Na⁺ in roots and shoots in T1 lines

The primary transgenics were grown to maturity in the glasshouse and were harvested for T1 seed. Two epidermal- and two xylem parenchyma-specific lines were chosen based on their adequate representation of the Na⁺-accumulating phenotype observed among all the primary transgenic lines and on the availability of T1 seed.

T1 lines were grown hydroponically for 10 d after transfer from petri dishes which were used to germinate the seed. At 10 d, 10 mM NaCl was added to the solution culture. After 10 d the YEB was harvested and Na⁺ and K⁺ contents of the YEB were determined using flame photometry. Both lines expressing PpENA1 in the xylem parenchyma cells accumulated less Na⁺ in the YEB than the WT control plants. Both lines expressing PpENA1 in the epidermal cells accumulated more Na⁺ in the YEB than the WT control plants. Immediately following harvest of the YEB, Na⁺ concentration was increased to 50 mM and the plants remained on this solution for an additional 10 d. Following this Na⁺ stress the YEB, the leaf blade immediately following the YEB harvested previously and a sample of approximately half the full-length roots were harvested. Roots were rinsed in RO prior to processing for flame photometry to remove any excess Na⁺ from the growth solution. The xylem parenchyma lines accumulated significantly more Na⁺ in the YEB, older leaf and root than the WT control. The epidermal lines accumulated significantly more Na⁺, as a group average, in the YEB, older leaf and root than the WT control. However, among the replicates of the two epidermal lines there were replicates with extremely high Na⁺ accumulation in the leaf blades and those with extremely low Na⁺ accumulation in the leaf blades (well below that of WT control).

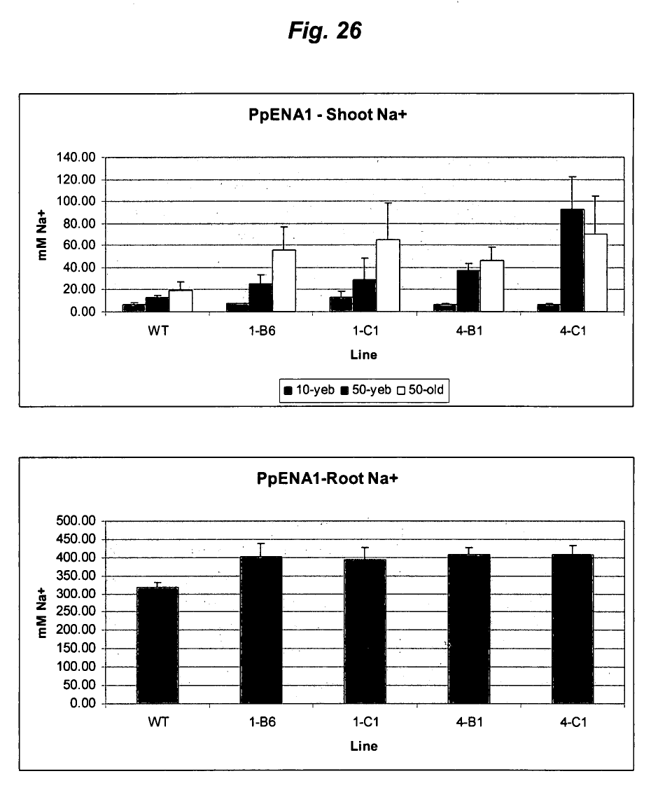

The results shown in FIG. 26 indicate that cell-specific expression of PpENA1 in rice roots can be used to alter levels of Na⁺ in roots and shoots. However, the reason for difference in phenotypes in the T1 lines selected remains to be determined.

Accordingly, the above results demonstrate that modulating accumulation of Na⁺ in a cell from a vascular plant may be achieved by expressing a Na⁺ pumping ATPase in the cell. In one embodiment, expressing a Na⁺ pumping ATPase in the cell may be used to decrease accumulation of Na⁺ in the cell. In an alternative embodiment, expressing a Na⁺ pumping ATPase in the cell may be used to increase accumulation of Na⁺ in the cell.

Finally, it will be appreciated that various modifications and variations of the described methods and compositions of the invention will be apparent to those skilled in the art without departing from the scope and spirit of the invention.

Although the invention has been described in connection with specific embodiments, it should be understood that the invention as claimed should not be unduly limited to such specific embodiments. Indeed, various modifications of the described modes for carrying out the invention which are apparent to those skilled in the art are intended to be within the scope of the present invention.

1. A vascular plant including cells expressing a Na⁺ pumping ATPase.

2. A plant according to claim 1, wherein the Na⁺ pumping ATPase is a moss Na⁺ pumping ATPase.

3. A plant according to claim 2, wherein the moss Na⁺ pumping ATPase is a Physcomitrella Na⁺ pumping ATPase.

4. A plant according to claim 3, wherein the Na⁺ pumping ATPase is expressed from the Physcomitrella patens ENA1 or ENA2 gene, or a variant thereof.

5. A plant according to claim 1, wherein expression of the Na⁺ pumping ATPase is driven from a constitutive promoter, a cell-specific promoter or a promoter up-regulated in response to Na⁺ stress.

6. A plant according to claim 1, wherein the cells include root cells and/or leaf trichome cells.

7. A plant according to claim 6, wherein the roots cells are selected from one or more of the group consisting of root epidermal cells, root cortex cells, and root stelar cells.

8. A plant according to claim 1, wherein the cells expressing a Na⁺ pumping ATPase have a reduced accumulation of Na⁺, as compared to similar cells not expressing a Na⁺ pumping ATPase.

9. A cell from a vascular plant, the cell expressing a Na⁺ pumping ATPase.

10. A cell according to claim 9, wherein the Na⁺ pumping ATPase is a moss Na⁺ pumping ATPase.

11. A cell according to claim 10, wherein the moss Na⁺ pumping ATPase is a Physcomitrella Na⁺ pumping ATPase.

12. A cell according to claim 11, wherein Na⁺ pumping ATPase is expressed from the Physcomitrella patens ENA1 or ENA2 gene, or a variant thereof.

13. A cell according to claim 9, wherein expression of the Na⁺ pumping ATPase is driven from a constitutive promoter, a cell-specific promoter or a promoter up-regulated in response to Na⁺ stress.

14. A cell according to claim 9, wherein the cell is a root cell or a leaf trichome cell.

15. A cell according to claim 14, wherein the root cell is selected from the group consisting of a root epidermal cell, a root cortex cell, and a root stelar cell.

16. A cell according to claim 9, wherein the cell has a reduced accumulation of Na⁺, as compared to a similar cell not expressing a Na⁺ pumping ATPase.

17. A method of decreasing accumulation of Na⁺ in a cell from a vascular plant, the method including the step of expressing a Na⁺ pumping ATPase in the cell and thereby decreasing accumulation of Na⁺ in the cell.

18. A method according to claim 17, wherein the Na⁺ pumping ATPase is a moss Na⁺ pumping ATPase.

19. A method according to claim 18, wherein the moss Na⁺ pumping ATPase is a Physcomitrella Na⁺ pumping ATPase.

20. A method according to claim 19, wherein expression of the Na⁺ pumping ATPase is from the Physcomitrella patens ENA1 or ENA2 gene, or a variant thereof.

21. A method according to claim 17, wherein expression of the Na⁺ pumping ATPase is driven from a constitutive promoter, a cell-specific promoter or a promoter up-regulated in response to Na⁺ stress.

22. A method according to claim 17, wherein the cell is a root cell or a leaf trichome cell.

23. A method according to claim 22, wherein the root cell is selected from the group consisting of a root epidermal cell, a root cortex cell, and a root stelar cell.

24. A plant cell produced according to the method of claim 17.

25. A plant, or a part of a plant, propagated from the plant cell according to claim 24.

26. A method of improving the tolerance to Na⁺ of a vascular plant, the method including the step of expressing a Na⁺ pumping ATPase in cells of the plant and thereby improving tolerance of the plant to Na⁺.

27. A plant produced according to claim 26.

28. A vascular plant with improved tolerance to Na⁺, the improved tolerance of the plant to Na⁺ due to expression of a Na⁺ pumping ATPase in cells of the plant.
